(19) United States
(12) Patent Application Publication
(10) Pub. No.: US 2025/0059540 A1
(43) Pub. Date: Feb. 20, 2025

(54) DOUBLE STRANDED RNA TARGETING ANGIOPOIETIN-LIKE 3 (ANGPTL-3) AND METHODS OF USE THEREOF

(71) Applicant: Sanegene Bio USA Inc., Woburn, MA (US)

(72) Inventors: Chunyang ZHANG, Burlington, MA (US); Shiyu WANG, Belmont, MA (US); Xiaochuan CAI, Woburn, MA (US); Weimin WANG, Winchester, MA (US); Zhongfa YANG, Sharon, MA (US)

(21) Appl. No.: 18/757,656

(22) Filed: Jun. 28, 2024

Related U.S. Application Data
(63) Continuation of application No. 18/057,186, filed on Nov. 18, 2022, now abandoned.
(60) Provisional application No. 63/281,253, filed on Nov. 19, 2021.

(51) Int. Cl. C12N 15/113; A61P 3/06

(52) U.S. Cl. CPC C12N 15/113; A61P 3/06; C12N 2310/11

(57) ABSTRACT

The disclosure relates to isolated oligonucleotides comprising duplex regions targeting angiopoietin-like 3 (ANGPTL-3), and delivery systems, kits and compositions comprising same, and methods of using same for inhibiting or downregulating ANGPTL-3.

### RELATED APPLICATIONS

This application is a continuation of U.S. application Ser. No. 18/057,186, filed Nov. 18, 2022, which claims priority to, and the benefit of, U.S. Provisional Application No. 63/281,253, filed Nov. 19, 2021. The entire contents of each of these applications are incorporated herein by reference.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing XML is “SANB-008_001US_SeqList_ST26”. The XML file is 561,421 bytes in size, created on Nov. 17, 2022.

### BACKGROUND

Angiopoietin-like 3 (ANGPTL3) is a member of the angiopoietin-like family of secreted factors that regulates lipid metabolism and that is predominantly expressed in the liver. ANGPTL3 dually inhibits the catalytic activities of lipoprotein lipase (LPL), which catalyzes the hydrolysis of triglycerides (TG), and of endothelial lipase (EL), which hydrolyzes high density lipoprotein (HDL) phospholipids. In addition, ANGPTL3 regulates plasma triglyceride (TRG) levels due to its inhibitory action on the activity of lipoprotein lipase (LPL), and genetic variants of ANGPTL-3 are associated with hypertriglyceridemia.

In some animal models of obesity, reduction in ANGPTL3 expression has a protective effect against hyperlipidemia and atherosclerosis by promoting the clearance of TG, and has been shown to also lead to a decrease in cholesterol and LDL levels in animal models. Mice deficient in ANGPTL3 have very low plasma TG and cholesterol levels, while overexpression produces the opposite effects. In humans, the plasma concentrations of ANGPTL3 correlate positively with plasma HDL cholesterol and HDL phospholipid levels. The observed effects of ANGPTL3 on lipid metabolism make it an attractive target for therapeutic intervention. Accordingly, there is an unmet need for highly potent and tolerable compounds to inhibit ANGPTL3. The present application discloses novel, highly potent inhibitors of ANGPTL3 expression and their use in treatment.

### SUMMARY

The present disclosure provides an isolated oligonucleotide comprising a sense strand and an anti-sense strand, wherein the sense strand comprises a nucleotide sequence that is substantially identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; l) 1353 to 1386; and m) 1407 to 1427, from the 5′ end of an Angiopoietin-like protein 3 (ANGPTL-3) mRNA sequence according to SEQ ID NO: 1, and the anti-sense strand is substantially complementary to the sense strand such that the sense strand and the anti-sense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; l) 1353 to 1386; and m) 1407 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; l) 1353 to 1386; and m) 1407 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 523 to 543; b) 680 to 700; and c) 1058 to 1078, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 523 to 543; b) 680 to 700; and c) 1058 to 1078, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 523 to 543; b) 680 to 700; and c) 1058 to 1078, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and l) 1353 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and l) 1353 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and l) 1353 to 1427, from the 5′ end of an ANGPTL-3 mRNA sequence according to SEQ ID NO: 1.

In some embodiments of the isolated oligonucleotide of the present disclosure, the sense strand comprises a sequence that substantially identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the sense strand comprises a sequence that is at least 70%, at least 80%, at least 90%, at least 95%, or at least 99% identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the sense strand comprises a sequence that is identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the isolated oligonucleotide is capable of inducing degradation of the ANGPTL-3 mRNA.

In some embodiments of the isolated oligonucleotide of the present disclosure, the sense strand is a single stranded RNA molecule. In some embodiments of the isolated oligonucleotide of the present disclosure, the anti-sense strand is a single stranded RNA molecule. In some embodiments of the isolated oligonucleotide of the present disclosure, both the sense strand and the anti-sense strand are single stranded RNA molecules.

In some embodiments of the isolated oligonucleotide of the present disclosure, the single stranded RNA molecule of the sense strand comprises a 3′ overhang. In some embodiments, in the single stranded RNA molecule of the sense strand, the 3′ overhang comprise at least one nucleotide. In some embodiments, in the single stranded RNA molecule of the sense strand, the 3′ overhang comprise two nucleotides.

In some embodiments of the isolated oligonucleotide of the present disclosure, the single stranded RNA molecule of the anti-sense strand comprises a 3′ overhang. In some embodiments, in the single stranded RNA molecule of the anti-sense strand, the 3′ overhang comprise at least one nucleotide. In some embodiments, in the single stranded RNA molecule of the anti-sense strand, the 3′ overhang comprise two nucleotides.

In some embodiments of the isolated oligonucleotide of the present disclosure, the 3′ overhang comprises any one of thymidine-thymidine (dTdT), Adenine-Adenine (AA), Cysteine-Cysteine (CC), Guanine-Guanine (GG) or Uracil-Uracil (UU).

In some embodiments of the isolated oligonucleotide of the present disclosure, the sense strand comprises an RNA sequence of at least 20 nucleotides in length. In some embodiments of the isolated oligonucleotide of the present disclosure, the sense strand comprises an RNA sequence of 20 nucleotides in length.

In some embodiments of the isolated oligonucleotide of the present disclosure, the anti-sense strand comprises an RNA sequence of at least 22 nucleotides in length. In some embodiments of the isolated oligonucleotide of the present disclosure, the anti-sense strand comprises an RNA sequence of 22 nucleotides in length.

In some embodiments of the isolated oligonucleotide of the present disclosure, the double stranded region is between 19 and 21 nucleotides in length. In some embodiments of the isolated oligonucleotide of the present disclosure, the double stranded region is 20 nucleotides in length.

In some embodiments of the isolated oligonucleotide of the present disclosure, the double stranded region comprises an anti-sense strand and a sense strand, according to any one of the pairs of anti-sense strand and sense strand sequences in Table 1, as described in the detailed description.

In some embodiments of the isolated oligonucleotide of the present disclosure, the anti-sense strand comprises a nucleotide sequence according to any one of: SEQ ID NOs: 2-62 and 200.

In some embodiments of the isolated oligonucleotide of the present disclosure, the sense strand comprises a nucleotide sequence according to any one of: SEQ ID NOs: 63-123 and 235.

In some embodiments of the isolated oligonucleotide of the present disclosure, the anti-sense strand comprises a nucleotide sequence according to any one of: SEQ ID NOs: 2-62 and 200; and the sense strand comprises a nucleotide sequence according to any one of: SEQ ID NOs: 63-123 and 235, wherein the anti-sense strand and the sense strand sequences have sufficient complementarity to allow formation of a double stranded region between the anti-sense and the sense strand.

In some embodiments of the isolated oligonucleotide of the present disclosure, wherein the sense strand comprises a nucleotide sequence that is identical to a region between any one of the nucleotide positions selected from: a) 523 to 543; b) 680 to 700; and c) 1058 to 1078, from the 5′ end of an ANGPTL-3 mRNA sequence according to SEQ ID NO: 1, the double stranded region comprises: i) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); ii) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); or iii) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′).

In some embodiments of the isolated oligonucleotide of the present disclosure, wherein the sense strand comprises a nucleotide sequence that is identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and l) 1353 to 1427, from the 5′ end of an ANGPTL-3 mRNA sequence according to SEQ ID NO: 1, the double stranded region comprises: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); iii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iv) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); v) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); vi) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′); vii) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); viii) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); ix) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); x) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); xi) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); xii) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xiii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiv) an anti-sense strand of nucleotide sequence according to SEQ ID NO: 22 (5′ UAUUUUUACAUCGUCUAACAUA 3′), and a sense strand of nucleotide sequence according to SEQ ID NO: 83 (5′ UGUUAGACGAUGUAAAAAUA 3′); xv) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); xvi) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); xvii) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xviii) an anti-sense strand of nucleotide sequence according to SEQ ID NO: 26 (5′ UAUAGAUCAUAAAAAGACUGAU 3′), and a sense strand of nucleotide sequence according to SEQ ID NO: 87 (5′ CAGUCUUUUUAUGAUCUAUA 3′); xix) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); xx) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); xxi) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xxii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); xxiii) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xxiv) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); xxv) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xxvi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xxvii) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); xxviii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xxix) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xxx) an anti-sense strand of nucleotide sequence according to SEQ ID NO: 38 (5′ UGAGAAAUUUCUGUGGGUUCUU 3′), and a sense strand of nucleotide sequence according to SEQ ID NO: 99 (5′ GAACCCACAGAAAUUUCUCA 3′); xxxi) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xxxii) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxxiii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xxxiv) an anti-sense strand of nucleotide sequence according to SEQ ID NO: 42 (5′ UAAACCAUAUUUGUAGUUCUCC 3′), and a sense strand of nucleotide sequence according to SEQ ID NO: 103 (5′ AGAACUACAAAUAUGGUUUA 3′); xxxv) an anti-sense strand of nucleotide sequence according to SEQ ID NO: 43 (5′ UUAAUUAGAUUGCUUCACUAUG 3′), and a sense strand of nucleotide sequence according to SEQ ID NO: 104 (5′ UAGUGAAGCAAUCUAAUUAA 3′); xxxvi) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xxxvii) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xxxviii) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′); or xxxix) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

In some embodiments of the isolated oligonucleotide of the present disclosure, wherein the sense strand comprises a sequence that is identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1, the double stranded region comprises: i) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); ii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iii) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); iv) an anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); v) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); vi) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); vii) an anti-sense strand of nucleotide sequence according to SEQ ID NO: 49 (5′ UUUUUACAUCGUCUAACAUAGC 3′), and a sense strand of nucleotide sequence according to SEQ ID NO: 110 (5′ UAUGUUAGACGAUGUAAAAA 3′); viii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); ix) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); x) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xi) an anti-sense strand of nucleotide sequence according to SEQ ID NO: 53 (5′ UAUGUAGUUCUUCUCAGUUCCU 3′), and a sense strand of nucleotide sequence according to SEQ ID NO: 114 (5′ GAACUGAGAAGAACUACAUA 3′); xii) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xiii) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xiv) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xv) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); xvi) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′); xvii) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xviii) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xix) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); or xx) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′).

In some embodiments, the isolated oligonucleotide of the present disclosure attenuates expression of the ANGPTL-3 mRNA by 20% to 50% (e.g., between 20% to 25%, 25% to 30%, 30% to 35%, 35% to 40%, 40% to 45% or 45% to 50%), at a dose of 0.02 nM.

In some embodiments of the isolated oligonucleotide of the present disclosure that attenuate expression of the ANGPTL-3 mRNA by 20% to 50%, at a dose of 0.02 nM, the double stranded region comprises: i) an anti-sense strand of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); ii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iii) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); iv) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); v) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); vi) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′); vii) an anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); viii) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′); ix) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); x) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); xi) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiii) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); xiv) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xv) an anti-sense strand of nucleotide sequence according to SEQ ID NO: 26 (5′ UAUAGAUCAUAAAAAGACUGAU 3′), and a sense strand of nucleotide sequence according to SEQ ID NO: 87 (5′ CAGUCUUUUUAUGAUCUAUA 3′); xvi) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); xvii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); xiii) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); xix) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xx) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); xxi) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xxii) an anti-sense strand of nucleotide sequence according to SEQ ID NO: 38 (5′ UGAGAAAUUUCUGUGGGUUCUU 3′), and an sense strand of nucleotide sequence according to SEQ ID NO: 99 (5′ GAACCCACAGAAAUUUCUCA 3′); xxiii) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xxiv) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxv) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xxvi) an anti-sense strand of nucleotide sequence according to SEQ ID NO: 42 (5′ UAAACCAUAUUUGUAGUUCUCC 3′), and an anti-sense strand of nucleotide sequence according to SEQ ID NO: 103 (5′ AGAACUACAAAUAUGGUUUA 3′); xxvii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); xxviii) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xxix) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); xxx) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xxxi) an anti-sense strand of nucleotide sequence according to SEQ ID NO: 49 (5′ UUUUUACAUCGUCUAACAUAGC 3′), and an anti-sense strand of nucleotide sequence according to SEQ ID NO: 110 (5′ UAUGUUAGACGAUGUAAAAA 3′); xxxii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); xxxiii) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); xxxiv) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xxxv) an anti-sense strand of nucleotide sequence according to SEQ ID NO: 53 (5′ UAUGUAGUUCUUCUCAGUUCCU 3′), and an anti-sense strand of nucleotide sequence according to SEQ ID NO: 114 (5′ GAACUGAGAAGAACUACAUA 3′); xxxvi) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xxxvii) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xxxviii) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xxxix) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′);

xL) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xLi) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xLii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xLiii) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xLiv) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); or xLv) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

In some embodiments, the isolated oligonucleotide of the present disclosure attenuates expression of the ANGPTL-3 mRNA by at least 50% (e.g., 50% to 55%, 55% to 60%, 60% to 65%, 65% to 70%, 70% to 75%, 75% to 80%, 80% to 85%, 85% to 90%, 90% to 95% or 95% to 99%, 99% to 100%), at a dose of 0.02 nM.

In some embodiments of the isolated oligonucleotide of the present disclosure that attenuate expression of the ANGPTL-3 mRNA by at least 50%, at a dose of 0.02 nM, the double stranded region comprises: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iii) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); iv) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); v) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); vi) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); vii) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); viii) an anti-sense strand of nucleotide sequence according to SEQ ID NO: 22 (5′ UAUUUUUACAUCGUCUAACAUA 3′), and a sense strand of nucleotide sequence according to SEQ ID NO: 83 (5′ UGUUAGACGAUGUAAAAAUA 3′); ix) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); x) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xi) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xii) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xiii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xiv) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xv) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); or xvi) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′).

In some embodiments, the isolated oligonucleotide of the present disclosure attenuates expression of the ANGPTL-3 mRNA by 50% to 75% (e.g., between 50% to 55%, 55% to 60%, 60% to 65%, 65% to 70% or 70% to 75%), at a dose of 0.1 nM.

In some embodiments of the isolated oligonucleotide of the present disclosure that attenuate expression of the ANGPTL-3 mRNA by 50% to 75%, at a dose of 0.1 nM, the double stranded region comprises: i) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); ii) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′); iii) anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); iv) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); v) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); vi) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); vii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); viii) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); ix) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); x) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xi) an anti-sense strand of nucleotide sequence according to SEQ ID NO: 38 (5′ UGAGAAAUUUCUGUGGGUUCUU 3′), and a sense strand of nucleotide sequence according to SEQ ID NO: 99 (5′ GAACCCACAGAAAUUUCUCA 3′); xii) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xiii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xiv) an anti-sense strand of nucleotide sequence according to SEQ ID NO: 42 (5′ UAAACCAUAUUUGUAGUUCUCC 3′), and a sense strand of nucleotide sequence according to SEQ ID NO: 103 (5′ AGAACUACAAAUAUGGUUUA 3′); xv) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xvi) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′); xvii) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xviii) an anti-sense strand of nucleotide sequence according to SEQ ID NO: 49 (5′ UUUUUACAUCGUCUAACAUAGC 3′), and an anti-sense strand of nucleotide sequence according to SEQ ID NO: 110 (5′ UAUGUUAGACGAUGUAAAAA 3′); xix) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xx) an anti-sense strand of nucleotide sequence according to SEQ ID NO: 53 (5′ UAUGUAGUUCUUCUCAGUUCCU 3′), and a sense strand of nucleotide sequence according to SEQ ID NO: 114 (5′ GAACUGAGAAGAACUACAUA 3′); xxi) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xxii) an anti-sense strand of nucleotide sequence according to SEQ ID NO: 60 (5′ UUUGUUGAAGUAGAAUUUUUUC 3′), and an anti-sense strand of nucleotide sequence according to SEQ ID NO: 121 (5′ AAAAAUUCUACUUCAACAAA 3′); xxiii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xxiv) an anti-sense strand of nucleotide sequence according to SEQ ID NO: 19 (5′ UCAUAGCAAAUCUUGAUUUUGG 3′), and an anti-sense strand of nucleotide sequence according to SEQ ID NO: 80 (5′ AAAAUCAAGAUUUGCUAUGA 3′); or xxv) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′).

In some embodiments, the isolated oligonucleotide of the present disclosure attenuates expression of the ANGPTL-3 mRNA by at least 75% (e.g., 75% to 80%, 805 to 85%, 85% to 90%, 90% to 95%, 95% to 99% or 99% to 100%), at a dose of 0.1 nM.

In some embodiments of the isolated oligonucleotide of the present disclosure that attenuates expression of the ANGPTL-3 mRNA by at least 75%, at a dose of 0.1 nM, the double stranded region comprises: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense strand of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); iii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iv) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); v) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); vi) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); vii) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); viii) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′);

ix) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); x) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); xi) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); xii) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xiii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiv) an anti-sense strand of nucleotide sequence according to SEQ ID NO: 22 (5′ UAUUUUUACAUCGUCUAACAUA 3′), and an anti-sense strand of nucleotide sequence according to SEQ ID NO: 83 (5′ UGUUAGACGAUGUAAAAAUA 3′); xv) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); xvi) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xvii) an anti-sense strand of nucleotide sequence according to SEQ ID NO: 26 (5′ UAUAGAUCAUAAAAAGACUGAU 3′), and an anti-sense strand of nucleotide sequence according to SEQ ID NO: 87 (5′ CAGUCUUUUUAUGAUCUAUA 3′); xviii) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xix) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xx) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xxi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xxii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xxiii) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxiv) an anti-sense strand of nucleotide sequence according to SEQ ID NO: 43 (5′ UUAAUUAGAUUGCUUCACUAUG 3′), and a sense strand of nucleotide sequence according to SEQ ID NO: 104 (5′ UAGUGAAGCAAUCUAAUUAA 3′); xxv) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xxvi) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); xxvii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); xxviii) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); xxix) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xxx) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xxxi) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); xxxii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); xxxiii) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xxxiv) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xxxv) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); xxxvi) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); or xxxvii) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

The present disclosure also provides a vector encoding an isolated oligonucleotide disclosed herein.

The present disclosure also provides a delivery system comprising an isolated oligonucleotide or vector disclosed herein.

The present disclosure also provides a pharmaceutical composition comprising an isolated oligonucleotide, vector or delivery system disclosed herein, and a pharmaceutically acceptable carrier, diluent or excipient.

The present disclosure also provides a kit comprising an isolated oligonucleotide, vector, delivery system or a pharmaceutical composition disclosed herein.

The present disclosure also provides a method of inhibiting or downregulating the expression or level of ANGPTL-3 in a subject in need thereof, wherein the method comprises administering to the subject an effective amount an isolated oligonucleotide, vector, delivery system or a pharmaceutical composition disclosed herein.

The present disclosure also provides a method of inhibiting or downregulating the expression or level of ANGPTL-3 in a subject in need thereof, wherein the method comprises administering to the subject an effective amount of a first and at least a second oligonucleotides disclosed herein, wherein the first and at least second oligonucleotides comprise different sequences.

### BRIEF DESCRIPTION OF THE DRAWINGS

FIG. 1 is a graph showing the efficacy of siRNA compounds listed in Table 2, Set 1 and Set 2, in silencing human Angpt13 in cultured Huh-7 cells at 0.02 nM. The compounds were transfected into cells at 0.02 nM concentration. Data is presented as % of human Angpt13 mRNA remaining relative to mock transfection when normalized to Gapdh mRNA levels (Mean, +/−SEM). Each bar represents a single compound tested, and the grid line on X-axis separates the two sets of compounds in Table 2, Set 1 and Set 2, respectively.

FIG. 2 is a graph showing the efficacy of siRNA compounds listed in Table 2, Set 1 and Set 2, in silencing human Angpt13 in cultured Huh-7 cells at 0.1 nM. The compounds were transfected into cells at 0.1 nM concentration. Data is presented as % of human Angpt13 mRNA remaining relative to mock transfection when normalized to Gapdh mRNA levels (Mean, +/−SEM). Each bar represents a single compound tested, and the grid line on X-axis separates the two sets of compounds in Table 2, Set 1 and Set 2, respectively.

FIGS. 3A-3R are graphs showing dose-response curves for indicated human Angpt13 siRNA compounds. The X-axis indicates the concentration (picomolar) of the indicated compounds that were transfected into Huh-7 cells. The IC₅₀ was calculated using the three parameter Log (inhibitor) vs. response formula and is indicated above each graph. The Y-axis indicates the % of human Angpt13 mRNA remaining relative to mock transfection when normalized to Gapdh mRNA levels (Mean, +/−SEM).

FIG. 4 is a graph showing in vivo potency of the indicated siRNA compounds, containing GalNAc conjugations, in silencing human Angpt13 in a BALB/c mouse model after administration of 0.5 mg/kg dosage of the indicated compound. Liver biopsies were taken on day 5 for mRNA expression analysis via RT-qPCR. Data is represented as % of human Angpt13 mRNA remaining relative to PBS groups when normalized to Neomycin-resistant (NeoR) gene mRNA levels (Mean, +/−SD).

FIG. 5 is a graph depicting in vivo dose response of the indicated siRNA compounds, containing GalNAc conjugations, in silencing human Angpt13 in a BALB/c mouse model after administration of 0.25 mg/kg (squares), 0.5 mg/kg (circles), and 1 mg/kg (triangles) of the indicated compound. Data is represented as % of human Angpt13 mRNA remaining relative to PBS groups when normalized to NeoR mRNA levels (Mean, +/−SD).

FIGS. 6A-6B are two graphs depicting in vivo compound potency and duration evaluations of the indicated siRNA compounds, containing chemical modifications and GalNAc conjugations, in a humanized transgenic mice model that expresses human Angplt3. Liver biopsies were taken on day 14 after compound administration for mRNA expression analysis via RT-qPCR. Serum hsANGPTL3 protein levels were analyzed on day −1, 4, 7, and 14 via ELISA. FIG. 6A shows percentage human Angpt13 mRNA remaining in the liver compared to PBS control. FIG. 6B shows absolute values of serum ANGPLT3 protein concentration compared to PBS control.

### DETAILED DESCRIPTION

The present disclosure provides isolated oligonucleotides (oligonucleotide(s)) that form a double stranded region, preferably small interfering RNAs (siRNAs), that can decrease ANGPTL-3 mRNA expression, in turn leading to a decrease in the degree of ANGPTL-3 protein expression in target cells. The oligonucleotides disclosed herein can have therapeutic application in regulating the expression of ANGPTL-3, for treatment of diseases, including but not limited to cardiovascular disease (CVD), atherosclerosis, hypercholesterolemia, hyperlipidemia or hypertriglyceridemia.

The present disclosure has identified specific regions within the ANGPTL-3 mRNA, that provide targets for binding double stranded oligonucleotides, e.g., siRNA, leading to reduction in level of expression of the ANGPTL-3 mRNA.

The ANGPTL-3 mRNA sequence described herein, is an mRNA sequence of ANGPTL-3 according to accession no. NM_014495.4:

```
(SEQ ID NO: 1)
      1 agaagaaaac agttccacgt tgcttgaaat tgaaaatcaa gataaaaatg ttcacaatta
    61 agctccttct ttttattgtt cctctagtta tttcctccag aattgatcaa gacaattcat
  121 catttgattc tctatctcca gagccaaaat caagatttgc tatgttagac gatgtaaaaa
  181 ttttagccaa tggcctcctt cagttgggac atggtcttaa agactttgtc cataagacga
  241 agggccaaat taatgacata tttcaaaaac tcaacatatt tgatcagtct ttttatgatc
  301 tatcgctgca aaccagtgaa atcaaagaag aagaaaagga actgagaaga actacatata
  361 aactacaagt caaaaatgaa gaggtaaaga atatgtcact tgaactcaac tcaaaacttg
  421 aaagcctcct agaagaaaaa attctacttc aacaaaaagt gaaatattta gaagagcaac
  481 taactaactt aattcaaaat caacctgaaa ctccagaaca cccagaagta acttcactta
  541 aaacttttgt agaaaaacaa gataatagca tcaaagacct tctccagacc gtggaagacc
  601 aatataaaca attaaaccaa cagcatagtc aaataaaaga aatagaaaat cagctcagaa
  661 ggactagtat tcaagaaccc acagaaattt ctctatcttc caagccaaga gcaccaagaa
  721 ctactccctt tcttcagttg aatgaaataa gaaatgtaaa acatgatggc attcctgctg
  781 aatgtaccac catttataac agaggtgaac atacaagtgg catgtatgcc atcagaccca
  841 gcaactctca agtttttcat gtctactgtg atgttatatc aggtagtcca tggacattaa
  901 ttcaacatcg aatagatgga tcacaaaact tcaatgaaac gtgggagaac tacaaatatg
  961 gttttgggag gcttgatgga gaattttggt tgggcctaga gaagatatac tccatagtga
1021 agcaatctaa ttatgtttta cgaattgagt tggaagactg gaaagacaac aaacattata
1081 ttgaatattc tttttacttg ggaaatcacg aaaccaacta tacgctacat ctagttgcga
1141 ttactggcaa tgtccccaat gcaatcccgg aaaacaaaga tttggtgttt tctacttggg
1201 atcacaaagc aaaaggacac ttcaactgtc cagagggtta ttcaggaggc tggtggtggc
1261 atgatgagtg tggagaaaac aacctaaatg gtaaatataa caaaccaaga gcaaaatcta
1321 agccagagag gagaagagga ttatcttgga agtctcaaaa tggaaggtta tactctataa
1381 aatcaaccaa aatgttgatc catccaacag attcagaaag ctttgaatga actgaggcaa
1441 atttaaaagg caataattta aacattaacc tcattccaag ttaatgtggt ctaataatct
1501 ggtattaaat ccttaagaga aagcttgaga aatagatttt ttttatctta aagtcactgt
1561 ctatttaaga ttaaacatac aatcacataa ccttaaagaa taccgtttac atttctcaat
1621 caaaattctt ataatactat ttgttttaaa ttttgtgatg tgggaatcaa ttttagatgg
1681 tcacaatcta gattataatc aataggtgaa cttattaaat aacttttcta aataaaaaat
1741 ttagagactt ttattttaaa aggcatcata tgagctaata tcacaacttt cccagtttaa
1801 aaaactagta ctcttgttaa aactctaaac ttgactaaat acagaggact ggtaattgta
1861 cagttcttaa atgttgtagt attaatttca aaactaaaaa tcgtcagcac agagtatgtg
1921 taaaaatctg taatacaaat ttttaaactg atgcttcatt ttgctacaaa ataatttgga
1981 gtaaatgttt gatatgattt atttatgaaa cctaatgaag cagaattaaa tactgtatta
2041 aaataagttc gctgtcttta aacaaatgga gatgactact aagtcacatt gactttaaca
2101 tgaggtatca ctatacctta tttgttaaaa tatatactgt atacatttta tatattttaa
2161 cacttaatac tatgaaaaca aataattgta aaggaatctt gtcagattac agtaagaatg
2221 aacatatttg tggcatcgag ttaaagttta tatttcccct aaatatgctg tgattctaat
2281 acattcgtgt aggttttcaa gtagaaataa acctcgtaac aagttactga acgtttaaac
2341 agcctgacaa gcatgtatat atgtttaaaa ttcaataaac aaagacccag tccctaaatt
2401 atagaaattt aaattattct tgcatgttta tcgacatcac aacagatccc taaatcccta
2461 aatccctaaa gattagatac aaatttttta ccacagtato acttgtcaga atttattttt
2521 aaatatgatt ttttaaaact gccagtaaga aattttaaat taaacccatt tgttaaagga
2581 tatagtgccc aagttatatg gtgacctacc tttgtcaata cttagcatta tgtatttcaa
2641 attatccaat atacatgtca tatatatttt tatatgtcac atatataaaa gatatgtatg
2701 atctatgtga atcctaagta aatattttgt tccagaaaag tacaaaataa taaaggtaaa
2761 aataatctat aattttcagg accacagact aagctgtcga aattaacgct gattttttta
2821 gggccagaat accaaaatgg ctcctctctt cccccaaaat tggacaattt caaatgcaaa
2881 ataattcatt atttaatata tgagttgctt cctctatttg gtttcc.
```

The present disclosure provides an isolated oligonucleotide comprising a sense strand and an anti-sense strand, wherein: the sense strand comprises a nucleotide sequence that is substantially identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; l) 1353 to 1386; and m) 1407 to 1427, from the 5′ end of an ANGPTL-3 mRNA sequence according to SEQ ID NO: 1, and the anti-sense strand is substantially complementary to the sense strand such that the sense strand and the anti-sense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; and l) 1353 to 1386; and m) 1407 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region comprising 19-25 nucleotides between any one of the nucleotide positions selected from: a) 52 to 93; b) 133 to 184; c) 246 to 304; d) 337 to 414; e) 433 to 503; f) 522 to 571; g) 617 to 700; h) 717 to 737; i) 836 to 905; j) 944 to 964; k) 1013 to 1093; and l) 1353 to 1386; and m) 1407 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 523 to 543; b) 680 to 700; and c) 1058 to 1078, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 523 to 543; b) 680 to 700; and c) 1058 to 1078; and i) 1353 to 1386, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 523 to 543; b) 680 to 700; and c) 1058 to 1078, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 522 to 542; b) 523 to 543; c) 1353 to 1373; d) 1361 to 1381; and e) 1366 to 1386, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 522 to 542; b) 523 to 543; c) 1353 to 1373; d) 1361 to 1381; and e) 1366 to 1386, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 522 to 542; b) 523 to 543; c) 1353 to 1373; d) 1361 to 1381; and e) 1366 to 1386, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and m) 1353 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and m) 1353 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and m) 1353 to 1427, from the 5′ end of an ANGPTL-3 mRNA sequence according to SEQ ID NO: 1.

In some embodiments of the isolated oligonucleotide of the present disclosure, the sense strand comprises a nucleotide sequence that is substantially identical to a region between any one of the nucleotide positions selected from: a) 52 to 72; b) 56 to 76, c) 57 to 77; d) 73 to 93; e) 138 to 158; f) 144 to 164; g) 133 to 153; h) 153 to 173; i) 161 to 181; j) 164 to 184; i) 275 to 295; j) 283 to 303; k) 284 to 304; l) 342 to 362; m) 345 to 365; e) 342 to 362; f) 357 to 377; g) 472 to 492; h) 473 to 493; i) 483 to 503; j) 453 to 473; k) 467 to 487; l) 548 to 568; m) 551 to 561; n) 679 to 699; o) 673 to 693; p) 717 to 737; q) 836 to 856; r) 885 to 905; s) 944 to 964; t) 1013 to 1033; u) 1060 to 1080; v) 1061 to 1081; and w) 1073 to 1093, from the 5′ end of an ANGPTL-3 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between any one of the nucleotide positions selected from: a) 52 to 72; b) 56 to 76, c) 57 to 77; d) 73 to 93; e) 138 to 158; f) 144 to 164; g) 133 to 153; h) 153 to 173; i) 161 to 181; j) 164 to 184; i) 275 to 295; j) 283 to 303; k) 284 to 304; l) 342 to 362; m) 345 to 365; e) 342 to 362; f) 357 to 377; g) 472 to 492; h) 473 to 493; i) 483 to 503; j) 453 to 473; k) 467 to 487; l) 548 to 568; m) 551 to 561; n) 679 to 699; o) 673 to 693; p) 717 to 737; q) 836 to 856; r) 885 to 905; s) 944 to 964; t) 1013 to 1033; u) 1060 to 1080; v) 1061 to 1081; and w) 1073 to 1093, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region between any one of the nucleotide positions selected from: a) 52 to 72; b) 56 to 76, c) 57 to 77; d) 73 to 93; e) 138 to 158; f) 144 to 164; g) 133 to 153; h) 153 to 173; i) 161 to 181; j) 164 to 184; i) 275 to 295; j) 283 to 303; k) 284 to 304; l) 342 to 362; m) 345 to 365; e) 342 to 362; f) 357 to 377; g) 472 to 492; h) 473 to 493; i) 483 to 503; j) 453 to 473; k) 467 to 487; l) 548 to 568; m) 551 to 561; n) 679 to 699; o) 673 to 693; p) 717 to 737; q) 836 to 856; r) 885 to 905; s) 944 to 964; t) 1013 to 1033; u) 1060 to 1080; v) 1061 to 1081; and w) 1073 to 1093, from the 5′ end of an ANGPTL-3 mRNA sequence according to SEQ ID NO: 1.

In some embodiments of the isolated oligonucleotide of the present disclosure, the sense strand comprises a sequence that substantially identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the sense strand comprises a sequence that is at least 70%, at least 80%, at least 90%, at least 95%, or at least 99% identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the sense strand comprises a sequence that is identical to a region comprising the sequence between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1.

In some embodiments of the isolated oligonucleotide of the present disclosure, the sense strand comprises a sequence that is substantially identical to a region between any one of the nucleotide positions selected from: a) 54 to 74; b) 158 to 178; c) 159 to 179; d) 148 to 168; e) 246 to 266; f) 262 to 282; g) 337 to 357; h) 341 to 361; i) 382 to 402; j) 394 to 414; k) 470 to 490; l) 475 to 495; m) 433 to 453; n) 435 to 455; o) 443 to 463; and p) 617 to 637, from the 5′ end of an ANGPTL-3 mRNA sequence according to SEQ ID NO: 1.

In some embodiments of the isolated oligonucleotide of the present disclosure, the sense strand comprises a sequence that is at least 70%, at least 80%, at least 90%, at least 95%, or at least 99% identical to a region between any one of the nucleotide positions selected from: a) 54 to 74; b) 158 to 178; c) 159 to 179; d) 148 to 168; e) 246 to 266; f) 262 to 282; g) 337 to 357; h) 341 to 361; i) 382 to 402; j) 394 to 414; k) 470 to 490; l) 475 to 495; m) 433 to 453; n) 435 to 455; o) 443 to 463; and p) 617 to 637, from the 5′ end of an ANGPTL-3 mRNA sequence according to SEQ ID NO: 1.

In some embodiments of the isolated oligonucleotide of the present disclosure, the sense strand comprises a sequence that is identical to a region between any one of the nucleotide positions selected from: a) 54 to 74; b) 158 to 178; c) 159 to 179; d) 148 to 168; e) 246 to 266; f) 262 to 282; g) 337 to 357; h) 341 to 361; i) 382 to 402; j) 394 to 414; k) 470 to 490; l) 475 to 495; m) 433 to 453; n) 435 to 455; o) 443 to 463; and p) 617 to 637, from the 5′ end of an ANGPTL-3 mRNA sequence according to SEQ ID NO: 1.

The ANGPTL-3 mRNA sequence according to SEQ ID NO: 1, as described herein, is any heterologous mRNA sequence with sufficient identity to an ANGPTL-3 according to accession no. NM_014495.4, as described herein, that allows binding to the sense strand of the oligonucleotides of the present disclosure.

In some embodiments of the isolated oligonucleotide of the present disclosure, the isolated oligonucleotide is capable of inducing degradation of the ANGPTL-3 mRNA.

In some embodiments of the isolated oligonucleotide of the present disclosure, the sense strand is a single stranded RNA molecule. In some embodiments of the isolated oligonucleotide of the present disclosure, the anti-sense strand is a single stranded RNA molecule. In some embodiments of the isolated oligonucleotide of the present disclosure, both the sense strand and the anti-sense strand are single stranded RNA molecules.

In some embodiments, the isolated oligonucleotide of the present disclosure is a small interfering RNA (siRNA). Accordingly, the disclosure provides siRNAs, wherein the siRNA comprises a sense region and anti-sense region complementary to the sense region that together form an RNA duplex, and wherein the sense region comprises a sequence at least 70% to 100% identical to a ANGPTL-3 mRNA sequence.

### Definitions

“RNAi” or “RNA interference” refers to the process of sequence-specific post-transcriptional gene silencing, mediated by double-stranded RNA (dsRNA). Duplex RNA siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information, etc. The anti-sense strand of these molecules can include RNA, DNA, PNA, or a combination thereof. These DNA/RNA chimera polynucleotide includes, but is not limited to, a double-strand polynucleotide composed of DNA and RNA that inhibits the expression of a target gene. These dsRNA molecules can also include one or more modified nucleotides, as described herein, which can be incorporated on either strand.

In the RNAi gene silencing or knockdown process, dsRNA comprising a first (anti-sense) strand that is complementary to a portion of a target gene and a second (sense) strand that is fully or partially complementary to the first anti-sense strand is introduced into an organism. After introduction into the organism, the target gene-specific dsRNA is processed into relatively small fragments (siRNAs) and can subsequently become distributed throughout the organism, decrease messenger RNA of target gene, leading to a phenotype that may come to closely resemble the phenotype arising from a complete or partial deletion of the target gene.

Certain dsRNAs in cells can undergo the action of Dicer enzyme, a ribonuclease III enzyme. Dicer can process the dsRNA into shorter pieces of dsRNA, i.e. siRNAs. RNAi also involves an endonuclease complex known as the RNA induced silencing complex (RISC). Following cleavage by Dicer, siRNAs enter the RISC complex and direct cleavage of a single stranded RNA target having a sequence complementary to the anti-sense strand of the siRNA duplex. The other strand of the siRNA is the passenger strand. Cleavage of the target RNA takes place in the middle of the region complementary to the anti-sense strand of the siRNA duplex. siRNAs can thus down regulate or knock down gene expression by mediating RNA interference in a sequence-specific manner.

As used herein, “target gene” or “target sequence” refers to a gene or gene sequence whose corresponding RNA is targeted for degradation through the RNAi pathway using dsRNAs or siRNAs as described herein. To target a gene, for example using an siRNA, the siRNA comprises an anti-sense region complementary to, or substantially complementary to, at least a portion of the target gene or sequence, and sense strand complementary to the anti-sense strand. Once introduced into a cell, the siRNA directs the RISC complex to cleave an RNA comprising a target sequence, thereby degrading the RNA.

As used herein, “oligonucleotide”, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an anti-sense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide of this disclosure. When dsRNA is produced synthetically, less common bases, such as inosine, 5-methylcytosine, 6-methyladenine, hypoxanthine and others can also be used for anti-sense, dsRNA, and ribozyme pairing. Other modifications, such as modification to the phosphodiester backbone, or the 2′-fluoro, the 2′-hydroxy or 2′O-methyl in the ribose sugar group of the RNA can also be made.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “region” or “fragment” is used interchangeably and as applied to an oligonucleotide.

The ANGPTL-3 mRNA sequence, as described herein, will be understood to mean a nucleotide sequence of reduced length relative to a reference nucleic acid or nucleotide sequence of the ANGPTL-3 mRNA sequence and comprising, consisting essentially of, and/or consisting of a nucleotide sequence of contiguous nucleotides identical or almost identical (e.g., 60%, 70%, 80%, 90%, 92%, 95%, 98% or 99% identical) to the reference nucleic acid or nucleotide sequence. Such a nucleic acid fragment according to the disclosure may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the disclosure.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the term “substantially complementary” is at least 90% (e.g., 91, 92, 93, 94, 95, 96, 97, 98 or 99%) complementary to the sense strand that is substantially identical to the nucleotide sequence within the defined regions in SEQ ID NO: 1. As used herein, the term “substantially complementary” means that two nucleic acid sequences are complementary at least at about 90%, 95% or 99% of their nucleotides.

In some embodiments, the two nucleic acid sequences can be complementary at least at 90%, 95%, 96%, 97%, 98%, 99% or more of their nucleotides. In some embodiments, the two nucleic acid sequences can be between 90% to 95% complementary, between 70% to 100% complementary, between 95% and 96% complementary, between 90% and 100% complementary, between 96% to 97% complementary, between 60% to 80% complementary, between 97% and 98% complementary, between 70% and 90% complementary, between 98% and 99% complementary, between 80% and 100% complementary, or between 99% and 100% complementary.

The term “substantially complementary” can also mean that two nucleic acid sequences, sense strand and anti-sense strand have sufficient complementarity that allows binding between the sense strand and anti-sense strand to form a double stranded region comprising of between 19-25 nucleotides in length. The term “substantially complementary” can also mean that two nucleic acid sequences can hybridize under high stringency conditions, and such conditions are well known in the art.

As used herein, the term “substantially identical” or “sufficient identity” used interchangeably herein, is at least 70%, at least 80%, at least 90%, at least 95%, or at least 99% (e.g., between 70% to 805, 8-% to 90% or 90% to 95% or 95% to 99% or 99% to 100%) identical to the nucleotide sequence within the defined regions in SEQ ID NO: 1.

As used herein, the term “identity” means that sequences are compared with one another as follows. In order to determine the percentage identity of two nucleic acid sequences, the sequences can first be aligned with respect to one another in order subsequently to make a comparison of these sequences possible. For this e.g., gaps can be inserted into the sequence of the first nucleic acid sequence and the nucleotides can be compared with the corresponding position of the second nucleic acid sequence. If a position in the first nucleic acid sequence is occupied by the same nucleotide as is the case at a position in the second sequence, the two sequences are identical at this position. The percentage identity between two sequences is a function of the number of identical positions divided by the number of all the positions compared in the sequences investigated.

A “percent identity” or “% identity” as used interchangeably herein, for aligned segments of a test sequence and a reference sequence is the percent of identical components which are shared by the two aligned sequences divided by the total number of components in reference sequence segment, i.e., the entire reference sequence or a smaller defined part of the reference sequence.

The percentage identity of two sequences can be determined with the aid of a mathematical algorithm. A preferred, but not limiting, example of a mathematical algorithm which can be used for comparison of two sequences is the algorithm of Karlin et al. (1993), PNAS USA, 90:5873-5877. Such an algorithm is integrated in the NBLAST program, with which sequences which have a desired identity to the sequences of the present disclosure can be identified. In order to obtain a gapped alignment, as described here, the “Gapped BLAST” program can be used, as is described in Altschul et al. (1997), Nucleic Acids Res, 25:3389-3402. If BLAST and Gapped BLAST programs are used, the preset parameters of the particular program (e.g. NBLAST) can be used. The sequences can be aligned further using version 9 of GAP (global alignment program) of the “Genetic Computing Group” using the preset (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of agreements as a percentage content of the nucleic acids in the sequence claimed. The methods described for determination of the percentage identity of two nucleic acid sequences can also be used correspondingly, if necessary, on the coded amino acid sequences.

Useful methods for determining sequence identity are also disclosed in Guide to Huge Computers (Martin J. Bishop, ed., Academic Press, San Diego (1994)), and Carillo, H., and Lipton, D., (Applied Math48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Percent identity can be 70% identity or greater, e.g., at least 70% identity, at least 75% identity, at least 80% identity, at least 85% identity, at least 90% identity, at least 95% identity, at least 98% identity, at least 99% identity or 100% identity.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

### Double Stranded RNAs Targeting ANGPTL-3

The disclosure provides isolated oligonucleotides comprising a double stranded RNAs (dsRNAs) duplex region which target a ANGPTL-3 mRNA sequence for degradation. The double stranded RNA molecule of the disclosure may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the dsRNA is a small interfering RNA (siRNA) which targets a ANGPTL-3 mRNA sequence for degradation. In some embodiments, the siRNA targeting ANGPTL-3 is packaged in a delivery system described herein (e.g., nanoparticle).

The isolated oligonucleotides of the present disclosure targeting ANGPTL-3 for degradation can comprise a sense strand at least 70% identical to any fragment of a ANGPTL-3 mRNA, for example the ANGPTL-3 mRNA of SEQ ID NO: 1. In some embodiments, the sense strand comprises or consists essentially of a sequence at least 70%, at least 80%, at least 90%, at least 95% or is 100% identical to any fragment of SEQ ID NO: 1. The siRNAs targeting ANGPTL-3 for degradation can comprise an anti-sense strand at least 70% identical to a sequence complementary to any fragment of a ANGPTL-3 mRNA, for example the ANGPTL-3 mRNA of SEQ ID NO: 1. In some embodiments, the anti-sense strand comprises or consists essentially of a sequence at least 70%, at least 80%, at least 90%, at least 95% or is 100% identical to a sequence complementary to any fragment of SEQ ID NO: 1. In some embodiments, the sense region and anti-sense regions are complementary, and base pair to form an RNA duplex structure. The fragment of the ANGPTL-3 mRNA that has percent identity to the sense region of the siRNA, and which is complementary to the anti-sense region of the siRNA, can be protein coding sequence of the mRNA, an untranslated region (UTR) of the mRNA (5′ UTR or 3′ UTR), or both.

In some embodiments, the isolated oligonucleotides of the present disclosure comprises a sense region and anti-sense region complementary to the sense region that together form an RNA duplex, and the sense region comprises a sequence at least 70% identical to a ANGPTL-3 mRNA sequence. In some embodiments, the sense region is identical to a ANGPTL-3 mRNA sequence.

As used herein, the term “sense strand” or “sense region” refers to a nucleotide sequence of an siRNA molecule that is partially or fully complementary to at least a portion of a corresponding anti-sense strand or anti-sense region of the siRNA molecule. The sense strand of an isolated oligonucleotides of the present disclosure molecule can include a nucleic acid sequence having some percentage identity with a target nucleic acid sequence such as a ANGPTL-3 mRNA sequence. In some cases, the sense region may have 100% identity, i.e. complete identity or homology, to the target nucleic acid sequence. In other cases, there may be one or more mismatches between the sense region and the target nucleic acid sequence. For example, there may be 1, 2, 3, 4, 5, 6, or 7 mismatches between the sense region and the target nucleic acid sequence.

As used herein, the term “anti-sense strand” or “anti-sense region” refers to a nucleotide sequence of the isolated oligonucleotides of the present disclosure, that is partially or fully complementary to at least a portion of a target nucleic acid sequence. The anti-sense strand of an isolated oligonucleotides of the present disclosure molecule can include a nucleic acid sequence that is complementary to at least a portion of a corresponding sense strand of the isolated oligonucleotides.

In some embodiments, the sense region comprises a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region consists essentially of a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region comprises a sequence that is identical to a sequence of SEQ ID NO: 1 or a region of SEQ ID NO: 1, as disclosed herein. In some embodiments, the sense region consists essentially of a sequence that is identical to a sequence of SEQ ID NO: 1 or a region of SEQ ID NO: 1, as disclosed herein.

In some embodiments, the sense region of the isolated oligonucleotides of the present disclosure targeting ANGPTL-3 has one or more mismatches between the sequence of the isolated oligonucleotides and the ANGPTL-3 sequence. For example, the sequence of the sense region may have 1, 2, 3, 4 or 5 mismatches between the sequence of the sense region of the isolated oligonucleotides and the ANGPTL-3 sequence. In some embodiments, the ANGPTL-3 sequence is an ANGPTL-3 3′ untranslated region sequence (3′ UTR). Without wishing to be bound by theory, it is thought that siRNAs targeting the 3′ UTR have elevated mismatch tolerance when compared to mismatches in the isolated oligonucleotides targeting coding regions of a gene. Further, the isolated oligonucleotides RNAs may be tolerant of mismatches outside the seed region. As used herein, the “seed region” of the isolated oligonucleotides refers to base pairs 2-8 of the anti-sense region of the isolated oligonucleotides, i.e., the strand of the isolated oligonucleotides that is complementary to and hybridizes to the target mRNA.

In some embodiments, the anti-sense region comprises a sequence that is at least 70% identical, at least 75% identical, at least 80% identical, at least 85% identical, at least 90% identical, at least 95% identical, at least 97% identical, at least 99% identical or 100% identical to a sequence complementary to a sequence of SEQ ID NO: 1 or a region of SEQ ID NO: 1, as disclosed herein. In some embodiments, the anti-sense region consists essentially of a sequence that is at least 70% identical, at least 75% identical, at least 80% identical, at least 85% identical, at least 90% identical, at least 95% identical, at least 97% identical, at least 99% or 100% identical to a sequence complementary to a sequence of SEQ ID NO: 1 or a region of SEQ ID NO: 1. In some embodiments, the anti-sense region comprises a sequence that is identical to a sequence complementary to a sequence of SEQ ID NO: 1 or a region of SEQ ID NO: 1. In some embodiments, the sense region consists essentially of a sequence that is complementary to a sequence of SEQ ID NO: 1 or a region of SEQ ID NO: 1.

The anti-sense region of the ANGPTL-3 targeting isolated oligonucleotide of the present disclosure is complementary to the sense region. In some embodiments, the sense region and the anti-sense region are fully complementary (no mismatches). In some embodiments the anti-sense region is partially complementary to the sense region, i.e., there are 1, 2, 3, 4 or 5 mismatches between the sense region and the anti-sense region.

In general, isolated oligonucleotide of the present disclosure comprise an RNA duplex that is about 16 to about 25 nucleotides in length. In some embodiments, the RNA duplex is between about 17 and about 24 nucleotides in length, between about 18 and about 23 nucleotides in length, or between about 19 and about 22 nucleotides in length. In some embodiments, the RNA duplex is 19 nucleotides in length. In some embodiments, the RNA duplex is 20 nucleotides in length.

In some embodiments of the isolated oligonucleotide of the present disclosure, the sense strand is a single stranded RNA molecule. In some embodiments of the isolated oligonucleotide of the present disclosure, the anti-sense strand is a single stranded RNA molecule. In some embodiments of the isolated oligonucleotide of the present disclosure, both the sense strand and the anti-sense strand are single stranded RNA molecules. In some embodiments of the isolated oligonucleotide of the present disclosure is an siRNA targeting ANGPTL-3, that comprises two different single stranded RNAs, the first comprising the sense region and the second comprising the anti-sense region, which hybridize to form an RNA duplex.

In some embodiments, the isolated oligonucleotide of the present disclosure, can have one or more overhangs from the duplex region. The overhangs, which are non-base-paired, single strand regions, can be from one to eight nucleotides in length, or longer. An overhang can be a 3′ overhang, wherein the 3′-end of a strand has a single strand region of from one to eight nucleotides. An overhang can be a 5′ overhang, wherein the 5′-end of a strand has a single strand region of from one to eight nucleotides.

The overhangs of the isolated oligonucleotide of the present disclosure can be the same length, or can be different lengths.

In some embodiments of the isolated oligonucleotide of the present disclosure, the single stranded RNA molecule of the sense strand comprises a 3′ overhang. In some embodiments, in the single stranded RNA molecule of the sense strand, the 3′ overhang comprise at least one nucleotide. In some embodiments, in the single stranded RNA molecule of the sense strand, the 3′ overhang comprise two nucleotides.

In some embodiments of the isolated oligonucleotide of the present disclosure, the single stranded RNA molecule of the anti-sense strand comprises a 3′ overhang. In some embodiments, in the single stranded RNA molecule of the anti-sense strand, the 3′ overhang comprise at least one nucleotide. In some embodiments, in the single stranded RNA molecule of the anti-sense strand, the 3′ overhang comprise two nucleotides.

In additional embodiments, both ends of isolated oligonucleotide of the present disclosure have an overhang, for example, a 3′ dinucleotide overhang on each end. The overhangs at the 5′- and 3′-ends may be of different lengths, or be the same length.

An overhang of an isolated oligonucleotide of the present disclosure can contain one or more deoxyribonucleotides, one or more ribonucleotides, or a combination of deoxyribonucleotides and ribonucleotides. In some embodiments, one, or both, of the overhang nucleotides of an siRNA may be 2′-deoxyribonucleotides.

In some embodiments, the first single stranded RNA molecule comprises a first 3′ overhang. In some embodiments, the second single stranded RNA molecule comprises a second 3′ overhang. In some embodiments, the first and second 3′ overhangs comprise a dinucleotide.

In some embodiments of the isolated oligonucleotide of the present disclosure, the 3′ overhang comprises any one of thymidine-thymidine (dTdT), Adenine-Adenine (AA), Cysteine-Cysteine (CC), Guanine-Guanine (GG) or Uracil-Uracil (UU). In some embodiments, the isolated oligonucleotide of the present disclosure, the 3′ overhang comprises a thymidine-thymidine (dTdT) or a Uracil-Uracil (UU) overhang. In some embodiment, the 3′ overhang comprises a Uracil-Uracil (UU) overhang. Without wishing to be bound by theory, it is thought that 3′ overhangs, such as dinucleotide overhangs, enhance siRNA mediated mRNA degradation by enhancing siRNA-RISC complex formation, and/or rate of cleavage of the target mRNA by the siRNA-RISC complex.

In some embodiments, the isolated oligonucleotide of the present disclosure can have one or more blunt ends, in which the duplex region ends with no overhang, and the strands are base paired to the end of the duplex region. In some embodiments, the isolated oligonucleotide of the present disclosure can have one or more blunt ends, or can have one or more overhangs, or can have a combination of a blunt end and an overhang end. For example, the 5′ end of the siRNA can be blunt and the 3′ end of the same isolated oligonucleotide comprise an overhang, or vice versa.

In some embodiments, both ends of the isolated oligonucleotide of the present disclosure are blunt ends.

In some embodiments of the isolated oligonucleotide of the present disclosure, the double stranded region comprises an anti-sense strand and a sense strand, according to any one of the pairs of anti-sense strand and sense strand sequences in Table 1, as described below.

TABLE 1
Exemplary Sequences of the Present Application

| SEQ | | Guide | Guide | SEQ | | Passenger | Passenger |
|---|---|---|---|---|---|---|---|
| ID | Anti-sense strand | start | end | ID | Sense strand | start | end |
| NO: | (guide) sequence | position | position | NO: | (passenger) sequence | position | position |
| 2 | UCAAUAAAAAGAAGGAGCUUAA | 58 | 78 | 63 | AAGCUCCUUCUUUUUAUUGA | 60 | 78 |
| 3 | UAAAUUUUUACAUCGUCUAACA | 163 | 183 | 64 | UUAGACGAUGUAAAAAUUUA | 165 | 183 |
| 4 | UAUAAAAAGACUGAUCAAAUAU | 276 | 296 | 65 | AUUUGAUCAGUCUUUUUAUA | 278 | 296 |
| 5 | UGACAUAUUCUUUACCUCUUCA | 377 | 397 | 66 | AAGAGGUAAAGAAUAUGUCA | 379 | 397 |
| 6 | UUUUAAGUGAAGUUACUUCUGG | 522 | 542 | 67 | AGAAGUAACUUCACUUAAAA | 524 | 542 |
| 7 | UUUUUAAGUGAAGUUACUUCUG | 523 | 543 | 68 | GAAGUAACUUCACUUAAAAA | 525 | 543 |
| 8 | UGAAGAUAGAGAAAUUUCUGUG | 680 | 700 | 69 | CAGAAAUUUCUCUAUCUUCA | 682 | 700 |
| 9 | UGAAAAACUUGAGAGUUGCUGG | 838 | 858 | 70 | AGCAACUCUCAAGUUUUUCA | 840 | 858 |
| 10 | UUAAUGUUUGUUGUCUUUCCAG | 1058 | 1078 | 71 | GGAAAGACAACAAACAUUAA | 1060 | 1078 |
| 11 | UGUAUAACCUUCCAUUUUGAGA | 1353 | 1373 | 72 | UCAAAAUGGAAGGUUAUACA | 1355 | 1373 |
| 12 | UUUUAUAGAGUAUAACCUUCCA | 1361 | 1381 | 73 | GAAGGUUAUACUCUAUAAAA | 1363 | 1381 |
| 13 | UUUGAUUUUAUAGAGUAUAACC | 1366 | 1386 | 74 | UUAUACUCUAUAAAAUCAAA | 1368 | 1386 |
| 14 | UAAAGAAGGAGCUUAAUUGUGA | 52 | 72 | 75 | ACAAUUAAGCUCCUUCUUUA | 54 | 72 |
| 15 | UAUAAAAAGAAGGAGCUUAAUU | 56 | 76 | 76 | UUAAGCUCCUUCUUUUUAUA | 58 | 76 |
| 16 | UAAUAAAAAGAAGGAGCUUAAU | 57 | 77 | 77 | UAAGCUCCUUCUUUUUAUUA | 59 | 77 |
| 17 | UAAAUAACUAGAGGAACAAUAA | 73 | 93 | 78 | AUUGUUCCUCUAGUUAUUUA | 75 | 93 |
| 18 | UAAAUCUUGAUUUUGGCUCUGG | 138 | 158 | 79 | AGAGCCAAAAUCAAGAUUUA | 140 | 158 |
| 19 | UCAUAGCAAAUCUUGAUUUUGG | 144 | 164 | 80 | AAAAUCAAGAUUUGCUAUGA | 146 | 164 |
| 20 | UUUGAUUUUGGCUCUGGAGAUA | 133 | 153 | 81 | UCUCCAGAGCCAAAAUCAAA | 135 | 153 |
| 21 | UAUCGUCUAACAUAGCAAAUCU | 153 | 173 | 82 | AUUUGCUAUGUUAGACGAUA | 155 | 173 |
| 22 | UAUUUUUACAUCGUCUAACAUA | 161 | 181 | 83 | UGUUAGACGAUGUAAAAAUA | 163 | 181 |
| 23 | UAAAAUUUUUACAUCGUCUAAC | 164 | 184 | 84 | UAGACGAUGUAAAAAUUUUA | 166 | 184 |
| 24 | UGAUAGAUCAUAAAAAGACUGA | 284 | 304 | 85 | AGUCUUUUUAUGAUCUAUCA | 286 | 304 |
| 25 | UUAAAAAGACUGAUCAAAUAUG | 275 | 295 | 86 | UAUUUGAUCAGUCUUUUUAA | 277 | 295 |
| 26 | UAUAGAUCAUAAAAAGACUGAU | 283 | 303 | 87 | CAGUCUUUUUAUGAUCUAUA | 285 | 303 |
| 27 | UUAGUUUAUAUGUAGUUCUUCU | 345 | 365 | 88 | AAGAACUACAUAUAAACUAA | 347 | 365 |
| 28 | UUUUAUAUGUAGUUCUUCUCAG | 342 | 362 | 89 | GAGAAGAACUACAUAUAAAA | 344 | 362 |
| 29 | UAUUUUUGACUUGUAGUUUAUA | 357 | 377 | 90 | UAAACUACAAGUCAAAAAUA | 359 | 377 |
| 30 | UUUAAGUUAGUUAGUUGCUCUU | 472 | 492 | 91 | GAGCAACUAACUAACUUAAA | 474 | 492 |
| 31 | UAUUAAGUUAGUUAGUUGCUCU | 473 | 493 | 92 | AGCAACUAACUAACUUAAUA | 475 | 493 |
| 32 | UUUGAUUUUGAAUUAAGUUAGU | 483 | 503 | 93 | UAACUUAAUUCAAAAUCAAA | 485 | 503 |
| 33 | UUUCUAAAUAUUUCACUUUUUG | 453 | 473 | 94 | AAAAGUGAAAUAUUUAGAAA | 455 | 473 |
| 34 | UUUAGUUAGUUGCUCUUCUAAA | 467 | 487 | 95 | UAGAAGAGCAACUAACUAAA | 469 | 487 |
| 35 | UCUAUUAUCUUGUUUUUCUACA | 548 | 568 | 96 | UAGAAAAACAAGAUAAUAGA | 550 | 568 |
| 36 | UAUGCUAUUAUCUUGUUUUUCU | 551 | 571 | 97 | AAAAACAAGAUAAUAGCAUA | 553 | 571 |
| 37 | UAAGAUAGAGAAAUUUCUGUGG | 679 | 699 | 98 | ACAGAAAUUUCUCUAUCUUA | 681 | 699 |
| 38 | UGAGAAAUUUCUGUGGGUUCUU | 673 | 693 | 99 | GAACCCACAGAAAUUUCUCA | 675 | 693 |
| 39 | UCUGAAGAAAGGGAGUAGUUCU | 717 | 737 | 100 | AACUACUCCCUUUCUUCAGA | 719 | 737 |
| 40 | UAAAACUUGAGAGUUGCUGGGU | 836 | 856 | 101 | CCAGCAACUCUCAAGUUUUA | 838 | 856 |
| 41 | UUUGAAUUAAUGUCCAUGGACU | 885 | 905 | 102 | UCCAUGGACAUUAAUUCAAA | 887 | 905 |
| 42 | UAAACCAUAUUUGUAGUUCUCC | 944 | 964 | 103 | AGAACUACAAAUAUGGUUUA | 946 | 964 |
| 43 | UUAAUUAGAUUGCUUCACUAUG | 1013 | 1033 | 104 | UAGUGAAGCAAUCUAAUUAA | 1015 | 1033 |
| 44 | UUAUAAUGUUUGUUGUCUUUCC | 1060 | 1080 | 105 | AAAGACAACAAACAUUAUAA | 1062 | 1080 |
| 45 | UAUAUAAUGUUUGUUGUCUUUC | 1061 | 1081 | 106 | AAGACAACAAACAUUAUAUA | 1063 | 1081 |
| 46 | UAAAGAAUAUUCAAUAUAAUGU | 1073 | 1093 | 107 | AUUAUAUUGAAUAUUCUUUA | 1075 | 1093 |
| 47 | UAAAAAGAAGGAGCUUAAUUGU | 54 | 74 | 108 | AAUUAAGCUCCUUCUUUUUA | 56 | 74 |
| 48 | UUUUACAUCGUCUAACAUAGCA | 158 | 178 | 109 | CUAUGUUAGACGAUGUAAAA | 160 | 178 |
| 49 | UUUUUACAUCGUCUAACAUAGC | 159 | 179 | 110 | UAUGUUAGACGAUGUAAAAA | 161 | 179 |
| 50 | UCUAACAUAGCAAAUCUUGAUU | 148 | 168 | 111 | UCAAGAUUUGCUAUGUUAGA | 150 | 168 |
| 51 | UUUGAAAUAUGUCAUUAAUUUG | 246 | 266 | 112 | AAUUAAUGACAUAUUUCAAA | 248 | 266 |
| 52 | UCAAAUAUGUUGAGUUUUUGAA | 262 | 282 | 113 | CAAAAACUCAACAUAUUUGA | 264 | 282 |
| 53 | UAUGUAGUUCUUCUCAGUUCCU | 337 | 357 | 114 | GAACUGAGAAGAACUACAUA | 339 | 357 |
| 54 | UUUAUAUGUAGUUCUUCUCAGU | 341 | 361 | 115 | UGAGAAGAACUACAUAUAAA | 343 | 361 |
| 55 | UCAAGUGACAUAUUCUUUACCU | 382 | 402 | 116 | GUAAAGAAUAUGUCACUUGA | 384 | 402 |
| 56 | UUUGAGUUGAGUUCAAGUGACA | 394 | 414 | 117 | UCACUUGAACUCAACUCAAA | 396 | 414 |
| 57 | UAAGUUAGUUAGUUGCUCUUCU | 470 | 490 | 118 | AAGAGCAACUAACUAACUUA | 472 | 490 |
| 58 | UGAAUUAAGUUAGUUAGUUGCU | 475 | 495 | 119 | CAACUAACUAACUUAAUUCA | 477 | 495 |
| 59 | UGUUGAAGUAGAAUUUUUUCUU | 433 | 453 | 120 | GAAAAAAUUCUACUUCAACA | 435 | 453 |
| 60 | UUUGUUGAAGUAGAAUUUUUUC | 435 | 455 | 121 | AAAAAUUCUACUUCAACAAA | 437 | 455 |
| 61 | UUUCACUUUUUGUUGAAGUAGA | 443 | 463 | 122 | UACUUCAACAAAAAGUGAAA | 445 | 463 |
| 62 | UUUUAUUUGACUAUGCUGUUGG | 617 | 637 | 123 | AACAGCAUAGUCAAAUAAAA | 619 | 637 |
| 200 | UUUCAAAGCUUUCUGAAUCUGU | 1407 | 1427 | 235 | AGAUUCAGAAAGCUUUGAAA | 1409 | 1427 |

In some embodiments, the sense region comprises a sequence selected from any one of the group of sense strand/passenger strand sequences listed in Table 1 or Table 2. In some embodiments, the anti-sense region comprises a sequence selected from any one of the group of anti-sense strand/guide strand sequences listed in Table 1 or Table 2. In some embodiments, the sense and anti-sense regions comprise complementary sequences selected from the group listed in Table 1 and Table 2.

In some embodiments of the isolated oligonucleotide of the present disclosure, the anti-sense strand comprises a nucleotide sequence according to any one of: SEQ ID NOs: 2-62 and 200.

In some embodiments of the isolated oligonucleotide of the present disclosure, the sense strand comprises a nucleotide sequence according to any one of: SEQ ID NOs: 63-123 and 235.

In some embodiments of the isolated oligonucleotide of the present disclosure, the anti-sense strand comprises a nucleotide sequence according to any one of: SEQ ID NOs: 2-62 and 200; and the sense strand comprises a nucleotide sequence according to any one of: SEQ ID NOs: 63-123 and 235, wherein the anti-sense strand and the sense strand sequences have sufficient complementarity to allow formation of a double stranded region between the anti-sense and the sense strand.

In some embodiments of the isolated oligonucleotide of the present disclosure, wherein the sense strand comprises a nucleotide sequence that is identical to a region between any one of the nucleotide positions selected from: a) 523 to 543; b) 680 to 700; and c) 1058 to 1078, from the 5′ end of an ANGPTL-3 mRNA sequence according to SEQ ID NO: 1, the double stranded region comprises any one of: i) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); ii) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); or iii) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′).

In some embodiments of the isolated oligonucleotide of the present disclosure, wherein the sense strand comprises a nucleotide sequence that is identical to a region between any one of the nucleotide positions selected from: a) 52 to 93; b) 133 to 184; c) 275 to 304; d) 342 to 377; e) 453 to 503; f) 548 to 571; g) 673 to 699; h) 717 to 737; i) 836 to 905; j) 944 to 964; k) 1013 to 1093; and m) 1353 to 1427, from the 5′ end of an ANGPTL-3 mRNA sequence according to SEQ ID NO: 1, the double stranded region comprises any one of: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); iii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iv) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); v) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); vi) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′); vii) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); viii) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); ix) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); x) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); xi) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); xii) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xiii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiv) an anti-sense strand of nucleotide sequence according to SEQ ID NO: 22 (5′ UAUUUUUACAUCGUCUAACAUA 3′), and a sense strand of nucleotide sequence according to SEQ ID NO: 83 (5′ UGUUAGACGAUGUAAAAAUA 3′); xv) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); xvi) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); xvii) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xviii) an anti-sense strand of nucleotide sequence according to SEQ ID NO: 26 (5′ UAUAGAUCAUAAAAAGACUGAU 3′), and a sense strand of nucleotide sequence according to SEQ ID NO: 87 (5′ CAGUCUUUUUAUGAUCUAUA 3′); xix) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); xx) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); xxi) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xxii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); xxiii) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xxiv) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); xxv) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xxvi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xxvii) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); xxviii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xxix) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xxx) an anti-sense strand of nucleotide sequence according to SEQ ID NO: 38 (5′ UGAGAAAUUUCUGUGGGUUCUU 3′), and a sense strand of nucleotide sequence according to SEQ ID NO: 99 (5′ GAACCCACAGAAAUUUCUCA 3′); xxxi) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xxxii) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxxiii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xxxiv) an anti-sense strand of nucleotide sequence according to SEQ ID NO: 42 (5′ UAAACCAUAUUUGUAGUUCUCC 3′), and a sense strand of nucleotide sequence according to SEQ ID NO: 103 (5′ AGAACUACAAAUAUGGUUUA 3′); xxxv) an anti-sense strand of nucleotide sequence according to SEQ ID NO: 43 (5′ UUAAUUAGAUUGCUUCACUAUG 3′), and a sense strand of nucleotide sequence according to SEQ ID NO: 104 (5′ UAGUGAAGCAAUCUAAUUAA 3′); xxxvi) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xxxvii) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xxxviii) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′); or xxxix) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

In some embodiments of the isolated oligonucleotide of the present disclosure, wherein the sense strand comprises a sequence that is identical to a region between any one of the nucleotide positions selected from: a) 54 to 74; b) 148 to 179; c) 246 to 282; d) 337 to 414; e) 433 to 495; and f) 522 to 542; g) 617 to 637; h) 838 to 858; and i) 1361 to 1381, from the 5′ end of an ANGPTL-3 mRNA sequence according to SEQ ID NO: 1, the double stranded region comprises any one of: i) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); ii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iii) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); iv) an anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); v) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); vi) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); vii) an anti-sense strand of nucleotide sequence according to SEQ ID NO: 49 (5′ UUUUUACAUCGUCUAACAUAGC 3′), and a sense strand of nucleotide sequence according to SEQ ID NO: 110 (5′ UAUGUUAGACGAUGUAAAAA 3′); viii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); ix) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); x) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xi) an anti-sense strand of nucleotide sequence according to SEQ ID NO: 53 (5′ UAUGUAGUUCUUCUCAGUUCCU 3′), and a sense strand of nucleotide sequence according to SEQ ID NO: 114 (5′ GAACUGAGAAGAACUACAUA 3′); xii) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xiii) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xiv) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xv) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); xvi) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′); xvii) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xviii) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xix) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); or xx) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′).

In some embodiments, the isolated oligonucleotide of the present disclosure attenuates expression of the ANGPTL-3 mRNA by 20% to 50%, at a dose of 0.02 nM.

In some embodiments of the isolated oligonucleotide of the present disclosure that attenuate expression of the ANGPTL-3 mRNA by 20% to 50%, at a dose of 0.02 nM, the double stranded region comprises any one of: i) an anti-sense strand of nucleotide sequence according to SEQ ID NO: 3 (5′ UAAAUUUUUACAUCGUCUAACA), and a sense strand of nucleotide sequence according to SEQ ID NO: 64 (5′ UUAGACGAUGUAAAAAUUUA 3′); ii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iii) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); iv) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); v) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); vi) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′); vii) an anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); viii) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′); ix) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); x) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); xi) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiii) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); xiv) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xv) an anti-sense strand of nucleotide sequence according to SEQ ID NO: 26 (5′ UAUAGAUCAUAAAAAGACUGAU 3′), and a sense strand of nucleotide sequence according to SEQ ID NO: 87 (5′ CAGUCUUUUUAUGAUCUAUA 3′); xvi) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); xvii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); xiii) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); xix) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xx) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); xxi) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xxii) an anti-sense strand of nucleotide sequence according to SEQ ID NO: 38 (5′ UGAGAAAUUUCUGUGGGUUCUU 3′), and an sense strand of nucleotide sequence according to SEQ ID NO: 99 (5′ GAACCCACAGAAAUUUCUCA 3′); xxiii) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xxiv) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxv) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xxvi) an anti-sense strand of nucleotide sequence according to SEQ ID NO: 42 (5′ UAAACCAUAUUUGUAGUUCUCC 3′), and an anti-sense strand of nucleotide sequence according to SEQ ID NO: 103 (5′ AGAACUACAAAUAUGGUUUA 3′); xxvii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); xxviii) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xxix) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); xxx) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xxxi) an anti-sense strand of nucleotide sequence according to SEQ ID NO: 49 (5′ UUUUUACAUCGUCUAACAUAGC 3′), and an anti-sense strand of nucleotide sequence according to SEQ ID NO: 110 (5′ UAUGUUAGACGAUGUAAAAA 3′); xxxii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); xxxiii) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); xxxiv) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xxxv) an anti-sense strand of nucleotide sequence according to SEQ ID NO: 53 (5′ UAUGUAGUUCUUCUCAGUUCCU 3′), and an anti-sense strand of nucleotide sequence according to SEQ ID NO: 114 (5′ GAACUGAGAAGAACUACAUA 3′); xxxvi) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xxxvii) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xxxviii) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xxxix) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′); xL) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xLi) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xLii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xLiii) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xLiv) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); or xLv) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

In some embodiments, the isolated oligonucleotide of the present disclosure attenuates expression of the ANGPTL-3 mRNA by at least 50%, at a dose of 0.02 nM.

In some embodiments of the isolated oligonucleotide of the present disclosure that attenuate expression of the ANGPTL-3 mRNA by at least 50%, at a dose of 0.02 nM, the double stranded region comprises any one of: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iii) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); iv) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); v) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); vi) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); vii) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); viii) an anti-sense strand of nucleotide sequence according to SEQ ID NO: 22 (5′ UAUUUUUACAUCGUCUAACAUA 3′), and a sense strand of nucleotide sequence according to SEQ ID NO: 83 (5′ UGUUAGACGAUGUAAAAAUA 3′); ix) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); x) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xi) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xii) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xiii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xiv) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xv) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); or xvi) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′).

In some embodiments, the isolated oligonucleotide of the present disclosure attenuates expression of the ANGPTL-3 mRNA by 50% to 75%, at a dose of 0.1 nM.

In some embodiments of the isolated oligonucleotide of the present disclosure that attenuate expression of the ANGPTL-3 mRNA by 50% to 75%, at a dose of 0.1 nM, the double stranded region comprises any one of: i) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); ii) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′); iii) anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); iv) an anti-sense strand of nucleotide sequence according to SEQ ID NO: 16 (5′ UAAUAAAAAGAAGGAGCUUAAU 3′), and a sense strand of nucleotide sequence according to SEQ ID NO: 77 (5′ UAAGCUCCUUCUUUUUAUUA 3′); v) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); vi) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); vii) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); viii) an anti-sense strand of nucleotide sequence according to SEQ ID NO: 32 (5′ UUUGAUUUUGAAUUAAGUUAGU 3′), and a sense strand of nucleotide sequence according to SEQ ID NO: 93 (5′ UAACUUAAUUCAAAAUCAAA 3′); ix) an anti-sense strand of nucleotide sequence according to SEQ ID NO: 35 (5′ UCUAUUAUCUUGUUUUUCUACA 3′), and a sense strand of nucleotide sequence according to SEQ ID NO: 96 (5′ UAGAAAAACAAGAUAAUAGA 3′); x) an anti-sense strand of nucleotide sequence according to SEQ ID NO: 37 (5′ UAAGAUAGAGAAAUUUCUGUGG 3′), and a sense strand of nucleotide sequence according to SEQ ID NO: 98 (5′ ACAGAAAUUUCUCUAUCUUA 3′); xi) an anti-sense strand of nucleotide sequence according to SEQ ID NO: 38 (5′ UGAGAAAUUUCUGUGGGUUCUU 3′), and a sense strand of nucleotide sequence according to SEQ ID NO: 99 (5′ GAACCCACAGAAAUUUCUCA 3′); xii) an anti-sense strand of nucleotide sequence according to SEQ ID NO: 39 (5′ UCUGAAGAAAGGGAGUAGUUCU 3′), and a sense strand of nucleotide sequence according to SEQ ID NO: 100 (5′ AACUACUCCCUUUCUUCAGA 3′); xiii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xiv) an anti-sense strand of nucleotide sequence according to SEQ ID NO: 42 (5′ UAAACCAUAUUUGUAGUUCUCC 3′), and a sense strand of nucleotide sequence according to SEQ ID NO: 103 (5′ AGAACUACAAAUAUGGUUUA 3′); xv) an anti-sense strand of nucleotide sequence according to SEQ ID NO: 44 (5′ UUAUAAUGUUUGUUGUCUUUCC 3′), and a sense strand of nucleotide sequence according to SEQ ID NO: 105 (5′ AAAGACAACAAACAUUAUAA 3′); xvi) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′); xvii) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xviii) an anti-sense strand of nucleotide sequence according to SEQ ID NO: 49 (5′ UUUUUACAUCGUCUAACAUAGC 3′), and an anti-sense strand of nucleotide sequence according to SEQ ID NO: 110 (5′ UAUGUUAGACGAUGUAAAAA 3′); xix) an anti-sense strand of nucleotide sequence according to SEQ ID NO: 52 (5′ UCAAAUAUGUUGAGUUUUUGAA 3′), and a sense strand of nucleotide sequence according to SEQ ID NO: 113 (5′ CAAAAACUCAACAUAUUUGA 3′); xx) an anti-sense strand of nucleotide sequence according to SEQ ID NO: 53 (5′ UAUGUAGUUCUUCUCAGUUCCU 3′), and a sense strand of nucleotide sequence according to SEQ ID NO: 114 (5′ GAACUGAGAAGAACUACAUA 3′); xxi) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xxii) an anti-sense strand of nucleotide sequence according to SEQ ID NO: 60 (5′ UUUGUUGAAGUAGAAUUUUUUC 3′), and an anti-sense strand of nucleotide sequence according to SEQ ID NO: 121 (5′ AAAAAUUCUACUUCAACAAA 3′); xxiii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xxiv) an anti-sense strand of nucleotide sequence according to SEQ ID NO: 19 (5′ UCAUAGCAAAUCUUGAUUUUGG 3′), and an anti-sense strand of nucleotide sequence according to SEQ ID NO: 80 (5′ AAAAUCAAGAUUUGCUAUGA 3′); or xxv) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′).

In some embodiments, the isolated oligonucleotide of the present disclosure attenuates expression of the ANGPTL-3 mRNA by at least 75%, at a dose of 0.1 nM.

In some embodiments of the isolated oligonucleotide of the present disclosure that attenuates expression of the ANGPTL-3 mRNA by at least 75%, at a dose of 0.1 nM, the double stranded region comprises any one of: i) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); ii) an anti-sense strand of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); iii) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); iv) an anti-sense strand of nucleotide sequence according to SEQ ID NO: 8 (5′ UGAAGAUAGAGAAAUUUCUGUG 3′), and a sense strand of nucleotide sequence according to SEQ ID NO: 69 (5′ CAGAAAUUUCUCUAUCUUCA 3′); v) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); vi) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); vii) an anti-sense strand of nucleotide sequence according to SEQ ID NO: 13 (5′ UUUGAUUUUAUAGAGUAUAACC 3′), and a sense strand of nucleotide sequence according to SEQ ID NO: 74 (5′ UUAUACUCUAUAAAAUCAAA 3′); viii) an anti-sense strand of nucleotide sequence according to SEQ ID NO: 14 (5′ UAAAGAAGGAGCUUAAUUGUGA 3′), and a sense strand of nucleotide sequence according to SEQ ID NO: 75 (5′ ACAAUUAAGCUCCUUCUUUA 3′); ix) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); x) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); xi) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); xii) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xiii) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xiv) an anti-sense strand of nucleotide sequence according to SEQ ID NO: 22 (5′ UAUUUUUACAUCGUCUAACAUA 3′), and an anti-sense strand of nucleotide sequence according to SEQ ID NO: 83 (5′ UGUUAGACGAUGUAAAAAUA 3′); xv) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); xvi) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xvii) an anti-sense strand of nucleotide sequence according to SEQ ID NO: 26 (5′ UAUAGAUCAUAAAAAGACUGAU 3′), and an anti-sense strand of nucleotide sequence according to SEQ ID NO: 87 (5′ CAGUCUUUUUAUGAUCUAUA 3′); xviii) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xix) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xx) an anti-sense strand of nucleotide sequence according to SEQ ID NO: 33 (5′ UUUCUAAAUAUUUCACUUUUUG 3′), and a sense strand of nucleotide sequence according to SEQ ID NO: 94 (5′ AAAAGUGAAAUAUUUAGAAA 3′); xxi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xxii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xxiii) an anti-sense strand of nucleotide sequence according to SEQ ID NO: 40 (5′ UAAAACUUGAGAGUUGCUGGGU 3′), and a sense strand of nucleotide sequence according to SEQ ID NO: 101 (5′ CCAGCAACUCUCAAGUUUUA 3′); xxiv) an anti-sense strand of nucleotide sequence according to SEQ ID NO: 43 (5′ UUAAUUAGAUUGCUUCACUAUG 3′), and a sense strand of nucleotide sequence according to SEQ ID NO: 104 (5′ UAGUGAAGCAAUCUAAUUAA 3′); xxv) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xxvi) an anti-sense strand of nucleotide sequence according to SEQ ID NO: 47 (5′ UAAAAAGAAGGAGCUUAAUUGU 3′), and a sense strand of nucleotide sequence according to SEQ ID NO: 108 (5′ AAUUAAGCUCCUUCUUUUUA 3′); xxvii) an anti-sense strand of nucleotide sequence according to SEQ ID NO: 50 (5′ UCUAACAUAGCAAAUCUUGAUU 3′), and a sense strand of nucleotide sequence according to SEQ ID NO: 111 (5′ UCAAGAUUUGCUAUGUUAGA 3′); xxviii) an anti-sense strand of nucleotide sequence according to SEQ ID NO: 51 (5′ UUUGAAAUAUGUCAUUAAUUUG 3′), and a sense strand of nucleotide sequence according to SEQ ID NO: 112 (5′ AAUUAAUGACAUAUUUCAAA 3′); xxix) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xxx) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xxxi) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); xxxii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); xxxiii) an anti-sense strand of nucleotide sequence according to SEQ ID NO: 59 (5′ UGUUGAAGUAGAAUUUUUUCUU 3′), and a sense strand of nucleotide sequence according to SEQ ID NO: 120 (5′ GAAAAAAUUCUACUUCAACA 3′); xxxiv) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xxxv) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA3′); xxxvi) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); or xxxvii) an anti-sense strand of nucleotide sequence according to SEQ ID NO: 200 (5′ UUUCAAAGCUUUCUGAAUCUGU 3′), and a sense strand of nucleotide sequence according to SEQ ID NO: 235 (5′ AGAUUCAGAAAGCUUUGAAA 3′).

The present disclosure provides an isolated oligonucleotide comprising a sense strand and an anti-sense strand, wherein: the sense strand comprises a nucleotide sequence that is substantially identical to a region comprising 19-25 nucleotides between any one of the nucleotide positions selected from: a) 9 to 103; b) 110 to 504; c) 522 to 571; d) 612 to 653; e) 670 to 767; f) 836 to 964; g) 988 to 1128; h) 1145 to 1218; i) 1248 to 1309; j) 1351 to 1456; and k) 1501 to 1521, from the 5′ end of an ANGPTL-3 mRNA sequence according to SEQ ID NO: 1, and the anti-sense strand is substantially complementary to the sense strand such that the sense strand and the anti-sense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region comprising 19-25 nucleotides between any one of the nucleotide positions selected from: a) 9 to 103; b) 110 to 504; c) 522 to 571; d) 612 to 653; e) 670 to 767; f) 836 to 964; g) 988 to 1128; h) 1145 to 1218; i) 1248 to 1309; j) 1351 to 1456; and k) 1501 to 1521, from the 5′ end of an ANGPTL-3 mRNA sequence according to SEQ ID NO: 1.

In some embodiments of the isolated oligonucleotide of the present disclosure, the sense strand comprises a nucleotide sequence that is identical to a region comprising 19-25 nucleotides between any one of the nucleotide positions selected from: a) 9 to 103; b) 110 to 504; c) 522 to 571; d) 612 to 653; e) 670 to 767; f) 836 to 964; g) 988 to 1128; h) 1145 to 1218; i) 1248 to 1309; j) 1351 to 1456; and k) 1501 to 1521, from the 5′ end of an ANGPTL-3 mRNA sequence according to SEQ ID NO: 1.

In some embodiments of the isolated oligonucleotide of the present disclosure, the anti-sense strand comprises a nucleotide sequence according to any one of: SEQ ID NOs: 2-62, 200, 268-411, and 523-633.

In some embodiments of the isolated oligonucleotide of the present disclosure, the sense strand comprises a nucleotide sequence according to any one of: SEQ ID NOs: 63-123, 235, 124-267, and 412-522.

In some embodiments of the isolated oligonucleotide of the present disclosure, the anti-sense strand comprises a nucleotide sequence according to any one of: SEQ ID NOs: 2-62, 200, 268-411, and 523-633; and the sense strand comprises a nucleotide sequence according to any one of: SEQ ID NOs: 63-123, 235, 124-267, and 412-522, wherein the anti-sense strand and the sense strand sequences have sufficient complementarity to allow formation of a double stranded region between the anti-sense and the sense strand.

In some embodiments, the isolated oligonucleotide of the present disclosure attenuates expression of the ANGPTL-3 mRNA in the liver by 20% to 50% (e.g., between 20% to 25%, 25% to 30%, 30% to 35%, 35% to 40%, 40% to 45% or 45% to 50%).

In some embodiments, the isolated oligonucleotide of the present disclosure attenuates expression of the ANGPTL-3 mRNA in the liver by 50 to 75% (e.g., between 50% to 55%, 55% to 60%, 60% to 65%, 65% to 70%, 70% to 75% or 75% to 80%).

In some embodiments, the isolated oligonucleotide of the present disclosure attenuates expression of the ANGPTL-3 mRNA in the liver by more than 75% (e.g., between 75% to 80%, 85% to 90%, 90% to 95%, 95% to 99%, or 99% to 100%).

In some embodiments of the isolated oligonucleotide of the present disclosure that attenuate expression of the ANGPTL-3 mRNA by 20% to 50%, 50% to 75%, or greater than 75% in the liver, the double stranded region comprises: i) an anti-sense of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); ii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); iv) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); v) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′); vi) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); vii) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); viii) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); ix) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); x) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xiii) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xiv) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xv) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xvi) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xvii) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xviii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xix) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xx) an anti-sense strand of nucleotide sequence according to SEQ ID NO: 278 (5′ UUAGCAAAUCUUGAUUUUGGCU 3′), and a sense strand of nucleotide sequence according to SEQ ID NO: 134 (5′ CCAAAAUCAAGAUUUGCUAA 3′); xxi) an anti-sense strand of nucleotide sequence according to SEQ ID NO: 558 (5′ UAGAAUUUUUUCUUCUAGGAGG 3′), and a sense strand of nucleotide sequence according to SEQ ID NO: 447 (5′ UCCUAGAAGAAAAAAUUCUA 3′); xxii) an anti-sense strand of nucleotide sequence according to SEQ ID NO: 7 (5′ UUUUUAAGUGAAGUUACUUCUG 3′), and a sense strand of nucleotide sequence according to SEQ ID NO: 68 (5′ GAAGUAACUUCACUUAAAAA 3′); xxiii) an anti-sense strand of nucleotide sequence according to SEQ ID NO: 2 (5′ UCAAUAAAAAGAAGGAGCUUAA 3′), and a sense strand of nucleotide sequence according to SEQ ID NO: 63 (5′ AAGCUCCUUCUUUUUAUUGA 3′); xxiv) an anti-sense strand of nucleotide sequence according to SEQ ID NO: 11 (5′ UGUAUAACCUUCCAUUUUGAGA 3′), and a sense strand of nucleotide sequence according to SEQ ID NO: 72 (5′ UCAAAAUGGAAGGUUAUACA 3′); xxv) an anti-sense strand of nucleotide sequence according to SEQ ID NO: 15 (5′ UAUAAAAAGAAGGAGCUUAAUU 3′), and a sense strand of nucleotide sequence according to SEQ ID NO: 76 (5′ UUAAGCUCCUUCUUUUUAUA 3′); xxvi) an anti-sense strand of nucleotide sequence according to SEQ ID NO: 17 (5′ UAAAUAACUAGAGGAACAAUAA 3′), and a sense strand of nucleotide sequence according to SEQ ID NO: 78 (5′ AUUGUUCCUCUAGUUAUUUA 3′); xxvii) an anti-sense strand of nucleotide sequence according to SEQ ID NO: 18 (5′ UAAAUCUUGAUUUUGGCUCUGG 3′), and a sense strand of nucleotide sequence according to SEQ ID NO: 79 (5′ AGAGCCAAAAUCAAGAUUUA 3′); xxviii) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); xxix) an anti-sense strand of nucleotide sequence according to SEQ ID NO: 20 (5′ UUUGAUUUUGGCUCUGGAGAUA 3′), and a sense strand of nucleotide sequence according to SEQ ID NO: 81 (5′ UCUCCAGAGCCAAAAUCAAA 3′); xxx) an anti-sense strand of nucleotide sequence according to SEQ ID NO: 21 (5′ UAUCGUCUAACAUAGCAAAUCU 3′), and a sense strand of nucleotide sequence according to SEQ ID NO: 82 (5′ AUUUGCUAUGUUAGACGAUA 3′); xxxi) an anti-sense strand of nucleotide sequence according to SEQ ID NO: 22 (5′ UAUUUUUACAUCGUCUAACAUA 3′), and a sense strand of nucleotide sequence according to SEQ ID NO: 83 (5′ UGUUAGACGAUGUAAAAAUA 3′); xxxii) an anti-sense strand of nucleotide sequence according to SEQ ID NO: 23 (5′ UAAAAUUUUUACAUCGUCUAAC 3′), and a sense strand of nucleotide sequence according to SEQ ID NO: 84 (5′ UAGACGAUGUAAAAAUUUUA 3′); xxxiii) an anti-sense strand of nucleotide sequence according to SEQ ID NO: 25 (5′ UUAAAAAGACUGAUCAAAUAUG 3′), and a sense strand of nucleotide sequence according to SEQ ID NO: 86 (5′ UAUUUGAUCAGUCUUUUUAA 3′); xxxiv) an anti-sense strand of nucleotide sequence according to SEQ ID NO: 26 (5′ UAUAGAUCAUAAAAAGACUGAU 3′), and a sense strand of nucleotide sequence according to SEQ ID NO: 87 (5′ CAGUCUUUUUAUGAUCUAUA 3′); xxxv) an anti-sense strand of nucleotide sequence according to SEQ ID NO: 28 (5′ UUUUAUAUGUAGUUCUUCUCAG 3′), and a sense strand of nucleotide sequence according to SEQ ID NO: 89 (5′ GAGAAGAACUACAUAUAAAA 3′); xxxvi) an anti-sense strand of nucleotide sequence according to SEQ ID NO: 29 (5′ UAUUUUUGACUUGUAGUUUAUA 3′), and a sense strand of nucleotide sequence according to SEQ ID NO: 90 (5′ UAAACUACAAGUCAAAAAUA 3′); xxxvii) an anti-sense strand of nucleotide sequence according to SEQ ID NO: 36 (5′ UAUGCUAUUAUCUUGUUUUUCU 3′), and a sense strand of nucleotide sequence according to SEQ ID NO: 97 (5′ AAAAACAAGAUAAUAGCAUA 3′); xxxviii) an anti-sense strand of nucleotide sequence according to SEQ ID NO: 43 (5′ UUAAUUAGAUUGCUUCACUAUG 3′), and a sense strand of nucleotide sequence according to SEQ ID NO: 104 (5′ UAGUGAAGCAAUCUAAUUAA 3′); xxxix) an anti-sense strand of nucleotide sequence according to SEQ ID NO: 45 (5′ UAUAUAAUGUUUGUUGUCUUUC 3′), and a sense strand of nucleotide sequence according to SEQ ID NO: 106 (5′ AAGACAACAAACAUUAUAUA 3′); xL) an anti-sense strand of nucleotide sequence according to SEQ ID NO: 46 (5′ UAAAGAAUAUUCAAUAUAAUGU 3′), and a sense strand of nucleotide sequence according to SEQ ID NO: 107 (5′ AUUAUAUUGAAUAUUCUUUA 3′); xLi) an anti-sense strand of nucleotide sequence according to SEQ ID NO: 6 (5′ UUUUAAGUGAAGUUACUUCUGG 3′), and a sense strand of nucleotide sequence according to SEQ ID NO: 67 (5′ AGAAGUAACUUCACUUAAAA 3′); xLii) an anti-sense strand of nucleotide sequence according to SEQ ID NO: 12 (5′ UUUUAUAGAGUAUAACCUUCCA 3′), and a sense strand of nucleotide sequence according to SEQ ID NO: 73 (5′ GAAGGUUAUACUCUAUAAAA 3′); xLiii) an anti-sense strand of nucleotide sequence according to SEQ ID NO: 57 (5′ UAAGUUAGUUAGUUGCUCUUCU 3′), and a sense strand of nucleotide sequence according to SEQ ID NO: 118 (5′ AAGAGCAACUAACUAACUUA 3′); xLiv) an anti-sense strand of nucleotide sequence according to SEQ ID NO: 58 (5′ UGAAUUAAGUUAGUUAGUUGCU 3′), and a sense strand of nucleotide sequence according to SEQ ID NO: 119 (5′ CAACUAACUAACUUAAUUCA 3′); xLv) an anti-sense strand of nucleotide sequence according to SEQ ID NO: 280 (5′ UGUCUAACAUAGCAAAUCUUGA 3′), and a sense strand of nucleotide sequence according to SEQ ID NO: 136 (5′ AAGAUUUGCUAUGUUAGACA 3′); xLvi) an anti-sense strand of nucleotide sequence according to SEQ ID NO: 296 (5′ UGACUGAUCAAAUAUGUUGAGU 3′), and a sense strand of nucleotide sequence according to SEQ ID NO: 152 (5′ UCAACAUAUUUGAUCAGUCA 3′); xLvii) an anti-sense strand of nucleotide sequence according to SEQ ID NO: 316 (5′ UAAUUUUTUCUUCUAGGAGGCU 3′), and a sense strand of nucleotide sequence according to SEQ ID NO: 172 (5′ CCUCCUAGAAGAAAAAAUUA 3′); or xLviii) an anti-sense strand of nucleotide sequence according to SEQ ID NO: 530 (5′ UUAAAAUUUUUACAUCGUCUAA 3′), and a sense strand of nucleotide sequence according to SEQ ID NO: 419 (5′ AGACGAUGUAAAAAUUUUAA 3′).

In some embodiments, the isolated oligonucleotide of the present disclosure attenuates expression of the ANGPTL-3 protein levels in serum by 20% to 50% (e.g., between 20% to 25%, 25% to 30%, 30% to 35%, 35% to 40%, 40% to 45% or 45% to 50%).

In some embodiments, the isolated oligonucleotide of the present disclosure attenuates expression of the ANGPTL-3 protein levels in serum by 50 to 75% (e.g., between 50% to 55%, 55% to 60%, 60% to 65%, 65% to 70%, 70% to 75% or 75% to 80%).

In some embodiments, the isolated oligonucleotide of the present disclosure attenuates expression of the ANGPTL-3 protein levels in serum by more than 75% (e.g., between 75% to 80%, 85% to 90%, 90% to 95%, 95% to 99%, or 99% to 100%).

In some embodiments of the isolated oligonucleotide of the present disclosure that attenuate expression of the ANGPTL-3 protein levels in serum by 20% to 50%, 50% to 75%, or greater than 75% in the liver, the double stranded region comprises: i) an anti-sense of nucleotide sequence according to SEQ ID NO: 3 (5′ UAAAUUUUUACAUCGUCUAACA 3′), and a sense strand of nucleotide sequence according to SEQ ID NO: 64 (5′ UUAGACGAUGUAAAAAUUUA 3′); ii) an anti-sense strand of nucleotide sequence according to SEQ ID NO: 4 (5′ UAUAAAAAGACUGAUCAAAUAU 3′), and a sense strand of nucleotide sequence according to SEQ ID NO: 65 (5′ AUUUGAUCAGUCUUUUUAUA 3′); iii) an anti-sense strand of nucleotide sequence according to SEQ ID NO: 5 (5′ UGACAUAUUCUUUACCUCUUCA 3′), and a sense strand of nucleotide sequence according to SEQ ID NO: 66 (5′ AAGAGGUAAAGAAUAUGUCA 3′); iv) an anti-sense strand of nucleotide sequence according to SEQ ID NO: 9 (5′ UGAAAAACUUGAGAGUUGCUGG 3′), and a sense strand of nucleotide sequence according to SEQ ID NO: 70 (5′ AGCAACUCUCAAGUUUUUCA 3′); v) an anti-sense strand of nucleotide sequence according to SEQ ID NO: 10 (5′ UUAAUGUUUGUUGUCUUUCCAG 3′), and a sense strand of nucleotide sequence according to SEQ ID NO: 71 (5′ GGAAAGACAACAAACAUUAA 3′);

vi) an anti-sense strand of nucleotide sequence according to SEQ ID NO: 19 (5′ UCAUAGCAAAUCUUGAUUUUGG 3′), and a sense strand of nucleotide sequence according to SEQ ID NO: 80 (5′ AAAAUCAAGAUUUGCUAUGA 3′); vii) an anti-sense strand of nucleotide sequence according to SEQ ID NO: 24 (5′ UGAUAGAUCAUAAAAAGACUGA 3′), and a sense strand of nucleotide sequence according to SEQ ID NO: 85 (5′ AGUCUUUUUAUGAUCUAUCA 3′); viii) an anti-sense strand of nucleotide sequence according to SEQ ID NO: 27 (5′ UUAGUUUAUAUGUAGUUCUUCU 3′), and a sense strand of nucleotide sequence according to SEQ ID NO: 88 (5′ AAGAACUACAUAUAAACUAA 3′); ix) an anti-sense strand of nucleotide sequence according to SEQ ID NO: 30 (5′ UUUAAGUUAGUUAGUUGCUCUU 3′), and a sense strand of nucleotide sequence according to SEQ ID NO: 91 (5′ GAGCAACUAACUAACUUAAA 3′); x) an anti-sense strand of nucleotide sequence according to SEQ ID NO: 31 (5′ UAUUAAGUUAGUUAGUUGCUCU 3′), and a sense strand of nucleotide sequence according to SEQ ID NO: 92 (5′ AGCAACUAACUAACUUAAUA 3′); xi) an anti-sense strand of nucleotide sequence according to SEQ ID NO: 34 (5′ UUUAGUUAGUUGCUCUUCUAAA 3′), and a sense strand of nucleotide sequence according to SEQ ID NO: 95 (5′ UAGAAGAGCAACUAACUAAA 3′); xii) an anti-sense strand of nucleotide sequence according to SEQ ID NO: 41 (5′ UUUGAAUUAAUGUCCAUGGACU 3′), and a sense strand of nucleotide sequence according to SEQ ID NO: 102 (5′ UCCAUGGACAUUAAUUCAAA 3′); xiii) an anti-sense strand of nucleotide sequence according to SEQ ID NO: 48 (5′ UUUUACAUCGUCUAACAUAGCA 3′), and a sense strand of nucleotide sequence according to SEQ ID NO: 109 (5′ CUAUGUUAGACGAUGUAAAA 3′); xiv) an anti-sense strand of nucleotide sequence according to SEQ ID NO: 54 (5′ UUUAUAUGUAGUUCUUCUCAGU 3′), and a sense strand of nucleotide sequence according to SEQ ID NO: 115 (5′ UGAGAAGAACUACAUAUAAA 3′); xv) an anti-sense strand of nucleotide sequence according to SEQ ID NO: 55 (5′ UCAAGUGACAUAUUCUUUACCU 3′), and a sense strand of nucleotide sequence according to SEQ ID NO: 116 (5′ GUAAAGAAUAUGUCACUUGA 3′); xvi) an anti-sense strand of nucleotide sequence according to SEQ ID NO: 56 (5′ UUUGAGUUGAGUUCAAGUGACA 3′), and a sense strand of nucleotide sequence according to SEQ ID NO: 117 (5′ UCACUUGAACUCAACUCAAA 3′); xvii) an anti-sense strand of nucleotide sequence according to SEQ ID NO: 60 (5′ UUUGUUGAAGUAGAAUUUUUUC 3′), and a sense strand of nucleotide sequence according to SEQ ID NO: 121 (5′ AAAAAUUCUACUUCAACAAA 3′); xviii) an anti-sense strand of nucleotide sequence according to SEQ ID NO: 61 (5′ UUUCACUUUUUGUUGAAGUAGA 3′), and a sense strand of nucleotide sequence according to SEQ ID NO: 122 (5′ UACUUCAACAAAAAGUGAAA 3′); xix) an anti-sense strand of nucleotide sequence according to SEQ ID NO: 62 (5′ UUUUAUUUGACUAUGCUGUUGG 3′), and a sense strand of nucleotide sequence according to SEQ ID NO: 123 (5′ AACAGCAUAGUCAAAUAAAA 3′); xx) an anti-sense strand of nucleotide sequence according to SEQ ID NO: 278 (5′ UUAGCAAAUCUUGAUUUUGGCU 3′), and a sense strand of nucleotide sequence according to SEQ ID NO: 134 (5′ CCAAAAUCAAGAUUUGCUAA 3′); or xxi) an anti-sense strand of nucleotide sequence according to SEQ ID NO: 558 (5′ UAGAAUUUUUUCUUCUAGGAGG 3′), and a sense strand of nucleotide sequence according to SEQ ID NO: 447 (5′ UCCUAGAAGAAAAAAUUCUA 3′).

In some embodiments, the isolated oligonucleotides of the present disclosure can comprises a linker, sometimes referred to as a loop. siRNAs comprising a linker or loop are sometimes referred to as short hairpin RNAs (shRNAs). In some embodiments, both the sense and the anti-sense regions of the siRNA are encoded by one single-stranded RNA. In these embodiments, and the anti-sense region and the sense region hybridize to form a duplex region. The sense and anti-sense regions are joined by a linker sequence, forming a “hairpin” or “stem-loop” structure. The siRNA can have complementary sense and anti-sense regions at opposing ends of a single stranded molecule, so that the molecule can form a duplex region with the complementary sequence portions, and the strands are linked at one end of the duplex region by a linker. The linker can be either a nucleotide or non-nucleotide linker or a combination thereof. The linker can interact with the first, and optionally, second strands through covalent bonds or non-covalent interactions.

Any suitable nucleotide linker sequence is envisaged as within the scope of the disclosure. An siRNA of this disclosure may include a nucleotide, non-nucleotide, or mixed nucleotide/non-nucleotide linker that joins the sense region of the nucleic acid to the anti-sense region of the nucleic acid. A nucleotide linker can be a linker of >2 nucleotides in length, for example about 3, 4, 5, 6, 7, 8, 9, 10, 11, 12, 13, 14, 15 or 16 nucleotides in length.

Examples of a non-nucleotide linker include an abasic nucleotide, polyether, polyamine, polyamide, peptide, carbohydrate, lipid, polyhydrocarbon, or other polymeric agents, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc, 1991, Vol. 113, pp. 6324-6326; Jaeschke et al., Tetrahedron Lett., 1993, Vol. 34, pp. 301; Arnold et al., WO 1989/002439; Usman et al., WO 1995/006731; Dudycz et al., WO 1995/011910, and Ferentz et al., J. Am. Chem. Soc, 1991, Vol. 113, pp. 4000-4002.

Examples of nucleotide linker sequences include, but are not limited to, AUG, CCC, UUCG, CCACC, AAGCAA, CCACACC and UUCAAGAGA.

In some embodiments, the isolated oligonucleotides of the present disclosure is an siRNA that can be a dsRNA of a length suitable as a Dicer substrate, which can be processed to produce a RISC active siRNA molecule. See, e.g., Rossi et al., US2005/0244858.

A Dicer substrate double stranded RNA (dsRNA) can be of a length sufficient that it is processed by Dicer to produce an active siRNA, and may further include one or more of the following properties: (i) the Dicer substrate dsRNA can be asymmetric, for example, having a 3′ overhang on the anti-sense strand, (ii) the Dicer substrate dsRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can from 19-30 bp in length.

In some embodiments of the isolated oligonucleotides of the present disclosure, the sense strand or the anti-sense strand or both comprise one or more modified nucleotide(s).

In some embodiments, the isolated oligonucleotides of the present disclosure comprises at least one modified nucleotide(s). In some embodiments, the one or more modified nucleotide(s) increases the stability or potency or both of the isolated oligonucleotide. In some embodiments, the one or more modified nucleotide(s) increases the stability of the RNA duplex, and siRNA.

Modifications that increase RNA stability include, but are not limited to locked nucleic acids. As used herein, the term “locked nucleic acid” or “LNA” includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. LNAs having a 2′-4′ cyclic linkage, as described in the International Patent Application WO 99/14226, WO 00/56746, WO 00/56748, and WO 00/66604, the contents of which are incorporated herein by reference.

In some embodiments, the one or more modified nucleotide comprises a phosphorothioate derivative or an acridinine substituted nucleotide. In some embodiments, the the isolated oligonucleotides of the present disclosure comprise a phosphate mimic at the 5′-terminus of antisense strand, including but not limited to vinyl-phosphonate or other phosphate analogues.

In some embodiments, the modified nucleotide comprises 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl-aminomethyluracil, 5-methoxyaminomethyl-2-thiouracil, beta-D-mannosylqueosine, 5′-methoxycarboxymethyluracil, 5-methoxyuracil, 2-methylthio-N-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2, 6-diaminopurine.

### Nucleic Acids and Vectors

The present disclosure also provides a vector encoding an isolated oligonucleotide disclosed herein. In some embodiments, the vector is any one of a plasmid, a cosmid or a viral vector. In some embodiments, the vector is an adenoviral vector. In some embodiments, the vector is a lentiviral vector. In some embodiments, the plasmid is an expression plasmid.

The disclosure provides nucleic acids comprising the sequences encoding the isolated oligonucleotides of the present disclosure (e.g., dsRNAs or siRNAs) targeting ANGPTL-3 described herein.

In some embodiments, the nucleic acids are ribonucleic acids (RNAs). In some embodiments, the nucleic acids are deoxyribonucleic acids (DNAs). The DNAs may be a vector or a plasmid, e.g., an expression vector.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

By the term “express” or “expression” of a polynucleotide coding sequence, it is meant that the sequence is transcribed, and optionally, translated. Typically, according to the present disclosure, expression of a coding sequence of the disclosure will result in production of the polypeptide of the disclosure. The entire expressed polypeptide or fragment can also function in intact cells without purification.

In some embodiments, the vector is an expression vector for manufacturing siRNAs of the disclosure. Exemplary expression vectors may comprise a sequence encoding the sense and/or anti-sense strand of the isolated oligonucleotide of the present disclosure, under the control of a suitable promoter for transcription. Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

The isolated oligonucleotide of the present disclosure (e.g., dsRNAs and siRNAs) can be expressed endogenously from plasmid or viral expression vectors, or from minimal expression cassettes, for example, PCR generated fragments comprising one or more promoters and an appropriate template or templates for transcribing the siRNA. Examples of commercially available plasmid-based expression vectors for shRNA include members of the pSilencer series (Ambion, Austin. Tex.) and pCpG-siRNA (InvivoGen. San Diego, Calif.). Examples of kits for production of PCR-generated shRNA expression cassettes include Silencer Express (Ambion, Austin, Tex.) and siXpress (Mirus, Madison. Wis.).

Viral vectors for the in vivo expression of the isolated oligonucleotides (e.g., siRNAs and dsRNAs) in eukaryotic cells are also contemplated as within the scope of the instant disclosure. Viral vectors may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the siRNA from the vector and methods of delivering the viral vector, for example incorporated within a nanoparticle, are within the ordinary skill of one in the art.

It will be apparent to those skilled in the art that any suitable vector, optionally incorporated into a nanoparticle, can be used to deliver the isolated oligonucleotides of the present disclosure (e.g., dsRNAs or siRNAs) described herein to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

### Delivery Systems

The present disclosure also provides a delivery system comprising the isolated oligonucleotide disclosed herein or vector of the present disclosure encoding an isolated oligonucleotide disclosed herein. In some embodiments, the delivery system is any one of a liposome, a nanoparticle, a polymer based delivery system or a ligand-conjugate delivery system. In some embodiments, the ligand-conjugate delivery system comprises one or more of an antibody, a peptide, a sugar moiety or a combination thereof.

In some embodiments, the delivery system of the present disclosure comprise nanoparticles comprising the isolated oligonucleotides of the present disclosure (e.g., siRNA or dsRNAs) targeting a ANGTL-3 mRNA for degradation.

In some embodiments, the nanoparticle comprises a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle is pH responsive. In some embodiments, the polymer-based nanoparticle further comprises a buffering component.

In some embodiments, the delivery system comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published Application 20090023673. In other embodiments, the one or more lipids are one or more cationic lipids. One skilled in the art will recognize which liposomes are appropriate for siRNA encapsulation.

In some embodiments, the liposome or the nanoparticle of the present disclosure comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a nanocrystal. Exemplary nanocrystals are crystalline particles with at least one dimension of less than 1000 nanometers, preferably of less than 100 nanometers.

In some embodiments, the nanoparticle comprises a polymer based nanoparticle. In some embodiments, the polymer comprises a multiblock copolymer, a diblock copolymer, a polymeric micelle or a hyperbranched macromolecule. In some embodiments, the particle comprises one or more cationic polymers. In some embodiments, the cationic polymer is chitosan, protamine, polylysine, polyhistidine, polyarginine or poly(ethylene)imine. In other embodiments, the one or more polymers contain the buffering component, degradable component, hydrophilic component, cleavable bond component or some combination thereof.

In some embodiments, the nanoparticles or some portion thereof are degradable. In other embodiments, the lipids and/or polymers of the nanoparticles are degradable.

In some embodiments, any of these delivery systems of the present disclosure can comprise a buffering component. In other embodiments, any of the of the present disclosure can comprise a buffering component and a degradable component. In still other embodiments, any of the of the present disclosure can comprise a buffering component and a hydrophilic component. In yet other embodiments, any of the of the present disclosure can comprise a buffering component and a cleavable bond component. In yet other embodiments, any of the of the present disclosure can comprise a buffering component, a degradable component and a hydrophilic component. In still other embodiments, any of the of the present disclosure can comprise a buffering component, a degradable component and a cleavable bond component. In further embodiments, any of the of the present disclosure can comprise a buffering component, a hydrophilic component and a cleavable bond component. In yet another embodiment, any of the of the present disclosure can comprise a buffering component, a degradable component, a hydrophilic component and a cleavable bond component. In some embodiments, the particle is composed of one or more polymers that contain any of the aforementioned combinations of components.

In some embodiments of the isolated oligonucleotides of the present disclosure, the delivery system comprises a ligand-conjugate delivery system. In some embodiments, the ligand-conjugate delivery system comprises one or more of an antibody, a peptide, a sugar moiety, lipid or a combination thereof

In further embodiments, the isolated oligonucleotide of the present disclosure targeting a ANGPTL-3 mRNA (e.g., siRNA or dsRNA) is conjugated to, complexed to, or encapsulated by the one or more lipids or polymers of the delivery system. In further embodiments, the isolated oligonucleotide of the present disclosure targeting a ANGPTL-3 mRNA (e.g., siRNA or dsRNA) can be encapsulated in the hollow core of a nanoparticle. Alternatively, or in addition, the isolated oligonucleotide of the present disclosure targeting a ANGPTL-3 mRNA (e.g., siRNA or dsRNA) can be incorporated into the lipid or polymer based shell of the delivery system, for example via intercalation. Alternatively, or in addition, the isolated oligonucleotide of the present disclosure targeting a ANGPTL-3 mRNA (e.g., siRNA or dsRNA) can be attached to the surface of the delivery system. In some embodiments, the isolated oligonucleotide of the present disclosure targeting a ANGPTL-3 mRNA (e.g., siRNA or dsRNA) is conjugated to one or more lipids or polymers of the delivery system, e.g. via covalent attachment.

In some embodiments, the ligand conjugate delivery system further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

In some embodiments, the targeting agent comprises a binding partner for a cell surface protein that is upregulated or overexpressed or normally expressed in a target cell encoding ANGPTL-3 mRNA and expressing ANGPTL-3 protein. In some embodiments, the binding partner can be a transmembrane peptidoglycan expressed on the surface of many types of such cells. Targeting of cell surface protein by the delivery system of the present disclosure thus provides superior delivery and specificity of the compositions of the disclosure to target cells. In some embodiments, the target cell can be any one of an intestinal cell, an arterial cell, a cell of the cardiovascular system, a hepatocyte, a pancreatic cell or a combination thereof.

In some embodiments, the delivery system of the present disclosure comprises a polymer based delivery system. In some embodiments, polymer based delivery system comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In other embodiments, the polyalkylene oxide is polyethylene oxide (PEO).

In some embodiments, the delivery system of the present disclosure is a polymer based nanoparticle. Polymer based nanoparticles comprise one or more polymers. In some embodiments, the one or more polymers comprise a polyester, poly(ortho ester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In some embodiments, the one or more polymers comprise poly(lactic-co-glycolic acid) (PLGA). In some embodiments, the one or more polymers comprise poly(lactic acid) (PLA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the polymer-based nanoparticle comprises poly(lactic-co-glycolic acid) PLGA polymers. In some embodiments, the PLGA nanoparticle further comprises a targeting agent, as described herein.

In some embodiments, the delivery system of the present disclosure is a nanoparticle of average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

### Therapeutic Agents

In some embodiments, the delivery system of the present disclosure are administered with one or more additional therapeutic agents. In some embodiments, the additional therapeutic agents can be a steroid, an anti-inflammatory agent, an antibody, a fusion protein, a small molecule or combination thereof.

In some embodiments, the additional therapeutic agent is incorporated into a delivery system of the present disclosure comprising at least one isolated oligonucleotide targeting ANGPTL-3, disclosed herein. In some embodiments, the additional therapeutic agent is conjugated to, complexed to, or encapsulated by the one or more lipids or polymers of the delivery system. Additional therapeutic agents can be encapsulated in the hollow core of delivery system. Alternatively, or in addition, Additional therapeutic agents can be incorporated into the lipid or polymer based shell of the delivery system, for example via intercalation. Alternatively, or in addition, additional therapeutic agents can be attached to the surface of the delivery system. In some embodiments, the additional therapeutic agents are conjugated to one or more lipids or polymers of the delivery system, e.g. via covalent attachment.

In some embodiments, the additional therapeutic agent and the delivery system at least one isolated oligonucleotide targeting ANGPTL-3, disclosed herein, are formulated in the same composition. For example, the delivery system comprising isolated oligonucleotide of the present disclosure targeting ANGPTL-3 and the additional therapeutic agent can be formulated in the same pharmaceutical composition.

In some embodiments, the additional therapeutic agent and the delivery system comprises at least one isolated oligonucleotide targeting ANGPTL-3, disclosed herein are formulated as separate compositions, e.g., for separate administration to a subject.

### Pharmaceutical Compositions

The present disclosure also provides a pharmaceutical composition comprising: an isolated oligonucleotide disclosed herein, a vector of the present disclosure encoding an isolated oligonucleotide disclosed herein, or a delivery system of the present disclosure, and a pharmaceutically acceptable carrier, diluent, or excipient.

The pharmaceutical compositions of the disclosure can optionally comprise therapeutic agents, pharmaceutical agents, carriers, adjuvants, dispersing agents, diluents, and the like.

In some embodiments, the pharmaceutical composition comprises a therapeutic agent, such as a chemotherapeutic agent. In some embodiments, the therapeutic agent is formulated in the delivery system comprising the one or more isolated oligonucleotides (e.g., dsRNA or siRNA) targeting ANGPTL-3 of the present disclosure.

In some embodiments, an additional therapeutic agent is not formulated in the delivery system comprising the one or more isolated oligonucleotides (e.g., dsRNA or siRNA) targeting ANGPTL-3 of the present disclosure, but both the delivery system and the therapeutic agent are formulated in the same pharmaceutical composition. In some embodiments, an additional therapeutic agent is not formulated in the delivery system comprising the one or more isolated oligonucleotides (e.g., dsRNA or siRNA) targeting ANGPTL-3 of the present disclosure, and the delivery system and the therapeutic agent are formulated in separate pharmaceutical compositions.

Pharmaceutical compositions can contain any of the reagents discussed above, and one or more of a pharmaceutically acceptable carrier, a diluent or an excipient.

A pharmaceutical composition is in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed agent) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a of this disclosure include powders, sprays, ointments, pastes, creams, lotions, gels, solutions, patches and inhalants. In one embodiment, the active agent is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), intraperitoneal (into the body cavity) and transmucosal administration. Solutions or suspensions used for parenteral, intradermal, intraperitoneal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

The pharmaceutical compositions containing the nanoparticles described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active agents into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required nanoparticle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible pharmaceutically acceptable carrier. They can be enclosed in gelatin capsules or compressed into tablets. For the purpose of oral therapeutic administration, the active age can be incorporated with excipients and used in the form of tablets, troches, or capsules. Oral compositions can also be prepared using a fluid carrier for use as a mouthwash, wherein the agents in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the agents are delivered in the form of an aerosol spray from pressured container or dispenser, which contains a suitable propellant, e.g., a gas such as carbon dioxide, or a nebulizer.

The pharmaceutical compositions of the present disclosure can be prepared with pharmaceutically acceptable carriers that will protect the one or more isolated oligonucleotides (e.g., dsRNAs or siRNAs) targeting ANGPTL-3 mRNA of the present disclosure against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, and the materials can be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms of the disclosure are dictated by and directly dependent on the unique characteristics of the active agent and the particular therapeutic effect to be achieved.

The pharmaceutical compositions can be included in a container, pack, or dispenser together with instructions for administration.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids.

Techniques for formulation and administration of the disclosed compositions of the disclosure can be found in Remington: the Science and Practice of Pharmacy, 19th edition, Mack Publishing Co., Easton, PA (1995).

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Methods of Making Isolated Oligonucleotides

Provided herein are methods of making the one or more oligonucleotides of (e.g., dsRNAs or siRNAs) targeting ANGPTL-3 of the present disclosure, and delivery systems comprising same.

The one or more oligonucleotides of (e.g., dsRNAs or siRNAs) targeting ANGPTL-3 of the present disclosure, may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers. The siRNAs can be purified by extraction with a solvent or resin, precipitation, electrophoresis, chromatography, or a combination thereof, for example. Alternatively, siRNAs may be used with little if any purification to avoid losses due to sample processing.

In some embodiments, the one or more oligonucleotides of (e.g., dsRNAs or siRNAs) targeting ANGPTL-3 of the present disclosure can be produced using an expression vector into which a nucleic acid encoding the double stranded RNA has been cloned, for example under control of a suitable promoter.

In some embodiments, the one or more oligonucleotides of (e.g., dsRNAs or siRNAs) targeting ANGPTL-3 of the present disclosure can be incorporated in a delivery system of the present disclosure (e.g., a nanoparticle).

Delivery systems comprising dsRNAs or siRNAs of the disclosure can be prepared by any suitable means known in the art. For example, polymeric nanoparticles can be prepared using various methods including, but not limited to, solvent evaporation, spontaneous emulsification, solvent diffusion, desolation, dialysis, ionic gelation, nanoprecipitation, salting out, spray drying and supercritical fluid methods. The dispersion of preformed polymers and the polymerization of monomers are two additional strategies for preparation of polymeric nanoparticles. However, the choice of an appropriate method depends upon various factors, which will be known to the person of ordinary skill in the art.

Sterile injectable solutions comprising a delivery system of the disclosure can be prepared by incorporating the one or more isolated oligonucleotides (e.g. dsRNA and siRNA) targeting ANGPTL-3 disclosed herein, in the delivery systems (e.g. nanoparticle) in the required amount in an appropriate solvent with one or a combination of ingredients enumerated herein, as required, followed by filtered sterilization. Alternatively, or in addition, sterilization can be achieved through other means such as radiation or gas. Generally, dispersions are prepared by incorporating the delivery particles into a sterile vehicle that contains a basic dispersion medium and the required other ingredients from those enumerated above. In the case of sterile powders for the preparation of sterile injectable solutions, methods of preparation are vacuum drying and freeze drying that yields a powder of delivery system comprising the one or more isolated oligonucleotides (e.g. dsRNA and siRNA) targeting ANGPTL-3 disclosed herein, plus any additional desired ingredient from a previously sterile filtered solution thereof.

### Methods of Use

The present disclosure also provides a method of inhibiting or downregulating the expression or level of ANGPTL-3 in a subject in need thereof, wherein the method comprises administering to the subject an effective amount an isolated oligonucleotide disclosed herein, a vector of the of the present disclosure encoding an isolated oligonucleotide disclosed herein, a delivery system of the present disclosure, or a pharmaceutical composition of the present disclosure.

The present disclosure also provides a method of treating or preventing a disease or disorder associated with aberrant or increased expression or activity of ANGPTL-3 or a disease or disorder where ANGPTL-3 plays a role in a subject in need thereof, wherein the method comprises administering to the subject an effective amount an isolated oligonucleotide disclosed herein, a vector of the of the present disclosure encoding an isolated oligonucleotide disclosed herein, a delivery system of the present disclosure, or a pharmaceutical composition of the present disclosure.

The present disclosure also provides an isolated oligonucleotide disclosed herein, a vector of the of the present disclosure encoding an isolated oligonucleotide disclosed herein, a delivery system of the present disclosure, or a pharmaceutical composition of the present disclosure, for use in treatment or prevention of a disease or disorder associated with aberrant or increased expression or activity of ANGPTL-3 or a disease or disorder where ANGPTL-3 plays a role, in a subject in need thereof.

The present disclosure also provides use of an isolated oligonucleotide disclosed herein, a vector of the of the present disclosure encoding an isolated oligonucleotide disclosed herein, a delivery system of the present disclosure, or a pharmaceutical composition of the present disclosure, in the manufacture of a medicament for treatment or prevention of a disease or disorder associated with aberrant or increased expression or activity of ANGPTL-3 or a disease or disorder where ANGPTL-3 plays a role in a subject in need thereof.

Provided herein are methods of inhibiting or downregulating ANGPTL-3 expression or activity in a cell, comprising contacting the cell with the one or more oligonucleotides (e.g., dsRNA or siRNA) targeting ANGPTL-3 as described herein. The one or more oligonucleotides (e.g., dsRNA or siRNA) targeting ANGPTL-3 as described herein can reduce or inhibit ANGPTL-3 activity through the RNAi pathway. The cell can be in vitro, in vivo or ex vivo. For example, the cell can be from a cell line, or in vivo in a subject in need thereof.

In some embodiments, the one or more oligonucleotides (e.g., dsRNA or siRNA) targeting ANGPTL-3 as described herein are capable of inducing RNAi-mediated degradation of an ANGPTL-3 mRNA in a cell of a subject.

As used herein, the terms “contacting,” “introducing” and “administering” are used interchangeably, and refer to a process by which dsRNA or siRNA of the present disclosure or a nucleic acid molecule encoding a dsRNA or siRNA of this disclosure is delivered to a cell, in order to inhibit or alter or modify expression of a target gene. The dsRNA may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

In contrast, the term “increase” or grammatical variations thereof as used herein refers to an increase or elevation in the specified level or activity of at least about 5%, about 10%, about 15%, about 25%, about 35%, about 40%, about 50%, about 60%, about 75%, about 80%, about 90%, about 95% or more. Increases in activity can be described in terms of fold change. For example, activity can be increased 1.2×, 1.5×, 2×, 3×, 5×, 6×, 7×, 8×, 9×, 10× or more compared to a baseline level of activity.

As used herein, the term “IC₅₀” or “IC₅₀ value” refers to the concentration of an agent where cell viability is reduced by half. The IC₅₀ is thus a measure of the effectiveness of an agent in inhibiting a biological process. In an exemplary model, cell lines are cultured using standard techniques, treated with any of the one or more oligonucleotides (e.g., dsRNA or siRNA) targeting ANGPTL-3 as described herein, and the IC₅₀ value of the oligonucleotides (e.g., dsRNA or siRNA) targeting ANGPTL-3 is calculated after 24, 48 and/or 72 hours to determine its effectiveness in downregulating or inhibiting the level of ANGPTL-3 mRNA or protein to 50%, as compared to the level of ANGPTL-3 mRNA or protein in an untreated cell or in the same cell before initiation of treatment with the isolated oligonucleotide.

Methods of monitoring of ANGPTL-3 mRNA and/or protein expression can be used to characterize gene silencing, and to determine the effectiveness of the compositions described herein. Expression of ANGPTL-3 may be evaluated by any known technique. Examples thereof include immunoprecipitations methods, utilizing ANGPTL-3 antibodies in assays such as ELISAs, Western Blot, or immunohistochemistry to visualize ANGPTL-3 protein expression in cells, or flow cytometry. Additional methods include various hybridization methods utilizing a nucleic acid that specifically hybridizes with a nucleic acid encoding ANGPTL-3 or a unique fragment thereof, or a transcription product (e.g., mRNA) or splicing product of said nucleic acid, Northern Blot methods, Southern blot methods, and various PCR-based methods such as RT-PCR, qPCR or digital droplet PCR. ANGPTL-3 mRNA expression may additionally be assessed using high throughput sequencing techniques.

Methods of assaying the effect of individual isolated oligonucleotides (e.g., dsRNA or siRNA) targeting ANGPTL-3 include transfecting representative cell lines with isolated oligonucleotides, and measuring viability. For example, cells from representative cell lines can be transfected using methods known in the art, such as the RNAiMAX Lipofectamine kit (Invitrogen, Carlsbad, CA), and cultured using any suitable technique known in the art. Optionally additional therapeutic agents as described herein can be added at variable concentrations to cell culture media following transfection. Following a suitable incubation period, such as 24-96 hours, cell viability can be measured using methods such as Cell Titer Glo 2.0 (Promega, CA) to determine cell viability, and/or ANGPTL-3 mRNA and protein levels can be assessed using the methods described herein.

In some embodiments of the methods of inhibiting or downregulating ANGPTL-3 expression or activity in a cell of the present disclosure, wherein the isolated oligonucleotide, the vector, the delivery system, or the pharmaceutical composition is administered parenterally.

In some embodiments of the methods of inhibiting or downregulating ANGPTL-3 expression or activity in a cell of the present disclosure, wherein the parenteral administration is intravenous, subcutaneous, intraperitoneal, or intramuscular.

In some embodiments of the methods of inhibiting or downregulating ANGPTL-3 expression or activity in a cell of the present disclosure, the subject is a human. In some embodiments of the methods of inhibiting or downregulating ANGPTL-3 expression or activity in a cell of the present disclosure, the subject has hypercholesterolemia, hypertriglyceridemia, coronary heart disease, peripheral arterial disease, stroke, type 2 diabetes or high blood pressure or a combination thereof.

In some embodiments of the methods of inhibiting or downregulating ANGPTL-3 expression or activity in a cell of the present disclosure, the method comprises administering the isolated oligonucleotide, the vector, the delivery system, or the pharmaceutical composition, in combination with at least a second therapeutic agent.

In some embodiments of the methods of inhibiting or downregulating ANGPTL-3 expression or activity in a cell of the present disclosure, the second therapeutic agent is an antibody, a small molecule drug, a peptide, a nucleotide molecule, or a combination thereof.

In some embodiments of the methods of inhibiting or downregulating ANGPTL-3 expression or activity in a cell of the present disclosure, the second therapeutic agent is an isolated oligonucleotide of the present disclosure.

The present disclosure also provides a method of inhibiting or downregulating the expression or level of ANGPTL-3 in a subject in need thereof, wherein the method comprises administering to the subject an effective amount of a first and at least a second oligonucleotides disclosed herein, wherein the first and at least second oligonucleotides comprise different sequences.

In some embodiments of the methods of inhibiting or downregulating ANGPTL-3 expression or activity in a cell of the present disclosure, wherein the first and at least second oligonucleotides are administered simultaneously.

In some embodiments of the methods of inhibiting or downregulating ANGPTL-3 expression or activity in a cell of the present disclosure, wherein the first and at least second oligonucleotides are administered sequentially.

In some embodiments of the methods of inhibiting or downregulating ANGPTL-3 expression or activity in a cell of the present disclosure, the subject is a human. In some embodiments of the methods of inhibiting or downregulating ANGPTL-3 expression or activity in a cell of the present disclosure, the subject has hypercholesterolemia, hypertriglyceridemia, coronary heart disease, peripheral arterial disease, stroke, type 2 diabetes or high blood pressure or a combination thereof.

In some embodiments of the method of treating or preventing a disease or disorder associated with aberrant or increased expression or activity of ANGPTL-3 or a disease or disorder where ANGPTL-3 plays a role of the present disclosure, the subject is a human. In some embodiments of the method of treating or preventing a disease or disorder associated with aberrant or increased expression or activity of ANGPTL-3 or a disease or disorder where ANGPTL-3 plays a role of the present disclosure, the disease or disorder is hypercholesterolemia, hypertriglyceridemia, coronary heart disease, peripheral arterial disease, stroke, type 2 diabetes or high blood pressure or a combination thereof.

In some embodiments of the use for treating or preventing a disease or disorder associated with aberrant or increased expression or activity of ANGPTL-3 or a disease or disorder where ANGPTL-3 plays a role of the present disclosure, the subject is a human. In some embodiments of the use for treating or preventing a disease or disorder associated with aberrant or increased expression or activity of ANGPTL-3 or a disease or disorder where ANGPTL-3 plays a role of the present disclosure, the disease or disorder is hypercholesterolemia, hypertriglyceridemia, coronary heart disease, peripheral arterial disease, stroke, type 2 diabetes or high blood pressure or a combination thereof.

In some embodiments of the use in the manufacture of a medicament for treatment or prevention of a disease or disorder associated with aberrant or increased expression or activity of ANGPTL-3 of the present disclosure, the subject is a human. In some embodiments of the use in the manufacture of a medicament for treatment or prevention of a disease or disorder associated with aberrant or increased expression or activity of ANGPTL-3 of the present disclosure of the present disclosure, the disease or disorder is hypercholesterolemia, hypertriglyceridemia, coronary heart disease, peripheral arterial disease, stroke, type 2 diabetes or high blood pressure or a combination thereof treatment or prevention of a disease or disorder associated with aberrant or increased expression or activity of ANGPTL-3.

### Routes of Administration

Nanoparticles comprising the one or more isolated oligonucleotides (e.g., dsRNA or siRNA) targeting ANGPTL-3 mRNA of the present disclosure can be administered to a subject by many of the well-known methods currently used for therapeutic treatment. For example, for treatment of mammalian diseases associated with expression or activity of ANGPTL-3, a compositions comprising the one or more isolated oligonucleotides (e.g., dsRNA or siRNA) targeting ANGPTL-3 mRNA of the present disclosure may be injected directly into cells, injected into the blood stream or body cavities or taken orally or applied through the skin with patches. The dose chosen should be sufficient to constitute effective treatment but not so high as to cause unacceptable side effects. The state of the disease condition and the health of the patient should preferably be closely monitored during and for a reasonable period after treatment.

The compositions comprising the one or more isolated oligonucleotides (e.g., dsRNA or siRNA) targeting ANGPTL-3 mRNA of the present disclosure can be administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the parenteral administration comprises intramuscular, intraperitoneal, subcutaneous or intravenous administration. One skilled in the art will recognize the advantages of certain routes of administration.

Compositions of the disclosure may be administered parenterally. Systemic administration of compositions comprising nanoparticles of the disclosure can also be by intravenous, transmucosal, subcutaneous, intraperitoneal, intramuscular or transdermal means. For intravenous parenteral administration, compositions comprising nanoparticles may be administered by injection or by infusion. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### Dosages

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing or treatment of the condition or symptom associated with expression or activity of ANGPTL-3. Dosages may vary depending on the age and size of the subject and the type and severity of the disease or disorder associated with ANGPTL-3 expression.

The term “effective amount” or “therapeutically effective amount”, as used interchangeably herein, refers to an amount of a pharmaceutical agent to treat, ameliorate, inhibit, downregulate or control the expression of ANGPTL-3 or symptoms associated with aberrant or abnormal expression of ANGPTL-3 in a subject, or to exhibit a detectable therapeutic or inhibitory effect in a subject. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any of the one or more isolated oligonucleotides (e.g., dsRNA or siRNA) targeting ANGPTL-3 mRNA of the present disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. In some embodiments, a standard xenograft or patient derived xenograft mouse model can be used to determine the effectiveness of the one or more isolated oligonucleotides (e.g., dsRNA or siRNA) targeting ANGPTL-3 mRNA of the present disclosure. Such information can then be used to determine useful doses and routes for administration in humans. Therapeutic/prophylactic efficacy and toxicity may be determined by standard pharmaceutical procedures in cell cultures or experimental animals, e.g., the maximum tolerated dose and no observable adverse effect dose. Pharmaceutical compositions that exhibit large therapeutic windows are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The dosage of nanoparticles comprising the one or more isolated oligonucleotides (e.g., dsRNA or siRNA) targeting ANGPTL-3 mRNA of the present disclosure, required depends on the choice of the route of administration; the nature of the formulation; the nature of the patient's illness; the subject's size, weight, surface area, age, and sex; other drugs being administered; and the judgment of the attending physician. Wide variations in the needed dosage are to be expected in view of the differing efficiencies of various routes of administration. For example, oral administration would be expected to require higher dosages than administration by intravenous injection (e.g., 2-, 3-, 4-, 6-, 8-, 10-; 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., capsules or implantable devices) may increase the efficiency of delivery, particularly for oral delivery.

A therapeutically effective dose of the one or more isolated oligonucleotides (e.g., dsRNA or siRNA) targeting ANGPTL-3 mRNA of the present disclosure, can optionally be combined with approved amounts of therapeutic agents, and described herein.

### Kits and Articles of Manufacture

The present disclosure also provides a kit comprising an isolated oligonucleotide disclosed herein, a vector of the present disclosure encoding an isolated oligonucleotide disclosed herein, a delivery system of the present disclosure, or a pharmaceutical composition of the present disclosure.

The kits are for use in the treatment of diseases related to abnormal or aberrant expression of ANGPTL-3, in a mammal. The kits are for use in downregulating or inhibiting expression of ANGPTL-3 partially or completely, in a mammal. In some embodiments, the mammal is a human, a mouse, a rat, a rabbit, a pig, a bovine, a canine, a feline, an ungulate, an ape, a monkey or an equine species. In some embodiments, the mammal is a human

Nanoparticles comprising the one or more isolated oligonucleotides (e.g., dsRNA or siRNA) targeting ANGPTL-3 mRNA of the present disclosure, can be lyophilized before being packaged in the kit, or can be provided in solution with a pharmaceutically acceptable carrier, diluent of excipient.

In some embodiments of the kits of the disclosure, the kit comprises a therapeutically effective amount of a composition comprising the delivery system of the present disclosure comprising one or more of the isolated oligonucleotides of the present disclosure targeting ANGPTL-3 (dsRNA or siRNA), and instructions for use of the same. In some embodiments, the kit further comprises at least one additional therapeutic agents, as described herein.

Articles of manufacture include, but are not limited to, instructions for use of the kit in treating diseases related to abnormal or aberrant expression of ANGPTL-3 or diseases related to expression of ANGPTL-3.

In some embodiments, the kits further comprise instructions for administering the isolated oligonucleotides, the vector, the delivery systems and the pharmaceutical compositions of the disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed invention. Based on the present disclosure the skilled artisan can identify and employ other components and methodology useful for practicing the present disclosure.

TABLE 2
Exemplary Sequences of the Present Application and Their Potency in vitro

| | | | | % of gene | | % of gene | |
|---|---|---|---|---|---|---|---|
| | Position | | | remaining | | remaining | |
| | of guide | Guide strand | Passenger strand | at 0.02 nM | | at 0.1 nM | |
| SEQ ID NOs | strand 3′ | sequence (5′-3′) | sequence (5′-3′) | Mean | SEM | Mean | SEM |

Set 1

| | | | | | | | |
|---|---|---|---|---|---|---|---|
| (SEQ ID NO: 268: | 30 | UUUUCAAGCAACGUGGAACUGU | AGUUCCACGUUGCUUGAAAA | 81.111533 | 9.3184535 | 32.645433 | 1.2792951 |
| SEQ ID NO: 124) | | | | | | | |
| (SEQ ID NO: 269: | 32 | UAAUUUCAAGCAACGUGGAACU | UUCCACGUUGCUUGAAAUUA | 90.014133 | 3.2787059 | 53.587933 | 3.6458452 |
| SEQ ID NO: 125) | | | | | | | |
| (SEQ ID NO: 270: | 35 | UUUCAAUUUCAAGCAACGUGGA | CACGUUGCUUGAAAUUGAAA | 80.0895 | 1.7664802 | 34.202767 | 3.7888977 |
| SEQ ID NO: 126) | | | | | | | |
| (SEQ ID NO: 271: | 36 | UUUUCAAUUUCAAGCAACGUGG | ACGUUGCUUGAAAUUGAAAA | 80.907933 | 3.3800739 | 38.524933 | 2.5584298 |
| SEQ ID NO: 127) | | | | | | | |
| (SEQ ID NO: 272: | 38 | UAUUUUCAAUUUCAAGCAACGU | GUUGCUUGAAAUUGAAAAUA | 86.937033 | 3.9987882 | 51.0402 | 3.5539255 |
| SEQ ID NO: 128) | | | | | | | |
| (SEQ ID NO: 273: | 39 | UGAUUUUCAAUUUCAAGCAACG | UUGCUUGAAAUUGAAAAUCA | 71.221633 | 2.6311449 | 37.2264 | 1.9406537 |
| SEQ ID NO: 129) | | | | | | | |
| (SEQ ID NO: 14: | 73 | UAAAGAAGGAGCUUAAUUGUGA | ACAAUUAAGCUCCUUCUUUA | 53.396667 | 1.438037 | 23.529267 | 0.5921558 |
| SEQ ID NO: 75) | | | | | | | |
| (SEQ ID NO: 47: | 75 | UAAAAAGAAGGAGCUUAAUUGU | AAUUAAGCUCCUUCUUUUUA | 52.968 | 1.0609924 | 22.5403 | 1.342357 |
| SEQ ID NO: 108) | | | | | | | |
| (SEQ ID NO: 274: | 82 | UGAACAAUAAAAAGAAGGAGCU | CUCCUUCUUUUUAUUGUUCA | 87.569467 | 3.0060246 | 53.4674 | 2.9598334 |
| SEQ ID NO: 130) | | | | | | | |
| (SEQ ID NO: 275: | 104 | UAUUCUGGAGGAAAUAACUAGA | UAGUUAUUUCCUCCAGAAUA | 71.2664 | 2.2272059 | 35.677733 | 1.013812 |
| SEQ ID NO: 131) | | | | | | | |
| (SEQ ID NO: 276: | 131 | UGAAUCAAAUGAUGAAUUGUCU | ACAAUUCAUCAUUUGAUUCA | 92.256833 | 3.0100584 | 58.2395 | 5.878688 |
| SEQ ID NO: 132) | | | | | | | |
| (SEQ ID NO: 277: | 158 | UAAUCUUGAUUUUGGCUCUGGA | CAGAGCCAAAAUCAAGAUUA | 75.575767 | 2.2352084 | 35.933667 | 0.5464555 |
| SEQ ID NO: 133) | | | | | | | |
| (SEQ ID NO: 18: | 159 | UAAAUCUUGAUUUUGGCUCUGG | AGAGCCAAAAUCAAGAUUUA | 43.241667 | 2.7841908 | 24.375967 | 5.3728394 |
| SEQ ID NO: 79) | | | | | | | |
| (SEQ ID NO: 278: | 163 | UUAGCAAAUCUUGAUUUUGGCU | CCAAAAUCAAGAUUUGCUAA | 56.316767 | 0.362523 | 30.630633 | 2.8476555 |
| SEQ ID NO: 134) | | | | | | | |
| (SEQ ID NO: 279: | 164 | UAUAGCAAAUCUUGAUUUUGGC | CAAAAUCAAGAUUUGCUAUA | 87.385267 | 3.893047 | 58.967033 | 9.1935964 |
| SEQ ID NO: 135) | | | | | | | |
| (SEQ ID NO: 19: | 165 | UCAUAGCAAAUCUUGAUUUUGG | AAAAUCAAGAUUUGCUAUGA | 45.484367 | 2.3409822 | 26.341767 | 4.0614731 |
| SEQ ID NO: 80) | | | | | | | |
| (SEQ ID NO: 280: | 171 | UGUCUAACAUAGCAAAUCUUGA | AAGAUUUGCUAUGUUAGACA | 64.104133 | 3.0918068 | 38.084767 | 5.5515859 |
| SEQ ID NO: 136) | | | | | | | |
| (SEQ ID NO: 48: | 179 | UUUUACAUCGUCUAACAUAGCA | CUAUGUUAGACGAUGUAAAA | 50.159233 | 4.767436 | 27.929767 | 4.1018037 |
| SEQ ID NO: 109) | | | | | | | |
| (SEQ ID NO: 49: | 180 | UUUUUACAUCGUCUAACAUAGC | UAUGUUAGACGAUGUAAAAA | 65.861167 | 0.9343576 | 30.1164 | 1.81541 |
| SEQ ID NO: 110) | | | | | | | |
| SEQ ID NO: 3: | 184 | UAAAUUUUUACAUCGUCUAACA | UUAGACGAUGUAAAAAUUUA | 53.736933 | 0.9785624 | 24.777 | 1.1407125 |
| SEQ ID NO: 64) | | | | | | | |
| (SEQ ID NO: 281: | 187 | UCUAAAAUUUUUACAUCGUCUA | GACGAUGUAAAAAUUUUAGA | 100.01487 | 2.9546284 | 66.843167 | 5.9206462 |
| SEQ ID NO: 137) | | | | | | | |
| (SEQ ID NO: 282: | 206 | UAACUGAAGGAGGCCAUUGGCU | CCAAUGGCCUCCUUCAGUUA | 97.9615 | 1.139691 | 54.3866 | 3.9999251 |
| SEQ ID NO: 138) | | | | | | | |
| (SEQ ID NO: 283: | 207 | UCAACUGAAGGAGGCCAUUGGC | CAAUGGCCUCCUUCAGUUGA | 99.696733 | 2.6662818 | 73.7278 | 2.6968611 |
| SEQ ID NO: 139) | | | | | | | |
| (SEQ ID NO: 284: | 217 | UGACCAUGUCCCAACUGAAGGA | CUUCAGUUGGGACAUGGUCA | 108.20177 | 5.5042258 | 92.5647 | 1.7211104 |
| SEQ ID NO: 140) | | | | | | | |
| (SEQ ID NO: 285: | 221 | UUUAAGACCAUGUCCCAACUGA | AGUUGGGACAUGGUCUUAAA | 93.7201 | 5.4120913 | 75.122933 | 6.4053112 |
| SEQ ID NO: 141) | | | | | | | |
| (SEQ ID NO: 286: | 223 | UCUUUAAGACCAUGUCCCAACU | UUGGGACAUGGUCUUAAAGA | 108.3241 | 6.3785735 | 102.46843 | 6.1372659 |
| SEQ ID NO: 142) | | | | | | | |
| (SEQ ID NO: 287: | 227 | UAAGUCUUUAAGACCAUGUCCC | GACAUGGuCUUAAAGACUUA | 100.31113 | 9.2975383 | 85.192233 | 3.1617187 |
| SEQ ID NO: 143) | | | | | | | |
| (SEQ ID NO: 288: | 228 | UAAAGUCUUUAAGACCAUGUCC | ACAUGGUCUUAAAGACUUUA | 102.038 | 8.0583773 | 88.811267 | 4.0530443 |
| SEQ ID NO: 144) | | | | | | | |
| (SEQ ID NO: 289: | 234 | UAUGGACAAAGUCUUUAAGACC | UCUUAAAGACUUUGUCCAUA | 98.124233 | 2.7328737 | 80.361867 | 6.4032413 |
| SEQ ID NO: 145) | | | | | | | |
| (SEQ ID NO: 290: | 236 | UUUAUGGACAAAGUCUUUAAGA | UUAAAGACUUUGUCCAUAAA | 97.103467 | 0.6574211 | 86.360567 | 3.0221867 |
| SEQ ID NO: 146) | | | | | | | |
| (SEQ ID NO: 291: | 262 | UAUAUGUCAUUAAUUUGGCCCU | GGCCAAAUUAAUGACAUAUA | 84.003533 | 0.6946951 | 55.887133 | 0.4712452 |
| SEQ ID NO: 147) | | | | | | | |
| (SEQ ID NO: 292: | 263 | UAAUAUGUCAUUAAUUUGGCCC | GCCAAAUUAAUGACAUAUUA | 63.596033 | 5.3027637 | 37.1758 | 1.0957327 |
| SEQ ID NO: 148) | | | | | | | |
| (SEQ ID NO: 293: | 264 | UAAAUAUGUCAUUAAUUUGGCC | CCAAAUUAAUGACAUAUUUA | 87.641767 | 2.0945498 | 64.418533 | 0.8667767 |
| SEQ ID NO: 149) | | | | | | | |
| (SEQ ID NO: 294: | 265 | UGAAAUAUGUCAUUAAUUUGGC | CAAAUUAAUGACAUAUUUCA | 85.362567 | 2.3767474 | 48.921933 | 2.6741333 |
| SEQ ID NO: 150) | | | | | | | |
| (SEQ ID NO: 295: | 275 | UUUGAGUUUUUGAAAUAUGUCA | ACAUAUUUCAAAAACUCAAA | 66.1322 | 1.6889226 | 35.774067 | 4.1253731 |
| SEQ ID NO: 151) | | | | | | | |
| (SEQ ID NO: 296: | 290 | UGACUGAUCAAAUAUGUUGAGU | UCAACAUAUUUGAUCAGUCA | 84.1016 | 2.7374969 | 51.850367 | 4.7386264 |
| SEQ ID NO: 152) | | | | | | | |
| (SEQ ID NO: 297: | 295 | UAAAAAGACUGAUCAAAUAUGU | AUAUUUGAUCAGUCUUUUUA | 72.8498 | 1.4487774 | 43.286567 | 2.4261051 |
| SEQ ID NO: 153) | | | | | | | |
| (SEQ ID NO: 24; | 305 | UGAUAGAUCAUAAAAAGACUGA | AGUCUUUUUAUGAUCUAUCA | 53.979033 | 1.905561 | 26.105867 | 1.6276034 |
| SEQ ID NO: 85) | | | | | | | |
| (SEQ ID NO: 298: | 328 | UCUUUGAUUUCACUGGUUUGCA | CAAACCAGUGAAAUCAAAGA | 86.6326 | 3.2865195 | 44.776033 | 4.4416589 |
| SEQ ID NO: 154) | | | | | | | |
| (SEQ ID NO: 299: | 333 | UUUCUUCUUUGAUUUCACUGGU | CAGUGAAAUCAAAGAAGAAA | 92.217433 | 5.5940638 | 59.213933 | 5.8075015 |
| SEQ ID NO: 155) | | | | | | | |
| (SEQ ID NO: 300: | 334 | UCUUCUUCUUUGAUUUCACUGG | AGUGAAAUCAAAGAAGAAGA | 69.148367 | 3.9724784 | 31.315 | 4.7646439 |
| SEQ ID NO: 156) | | | | | | | |
| (SEQ ID NO: 301: | 336 | UUUCUUCUUCUUUGAUUUCACU | UGAAAUCAAAGAAGAAGAAA | 87.131667 | 5.427754 | 58.7004 | 6.4070105 |
| SEQ ID NO: 157) | | | | | | | |
| (SEQ ID NO: 302: | 338 | UUUUUCUUCUUCUUUGAUUUCA | AAAUCAAAGAAGAAGAAAAA | 92.6884 | 12.279077 | 34.9207 | 19.948282 |
| SEQ ID NO: 158) | | | | | | | |
| (SEQ ID NO: 303: | 343 | UGUUCCUUUUCUUCUUCUUUGA | AAAGAAGAAGAAAAGGAACA | 98.193767 | 5.2532341 | 63.4527 | 4.7471188 |
| SEQ ID NO: 159) | | | | | | | |
| (SEQ ID NO: 304: | 347 | UCUCAGUUCCUUUUCUUCUUCU | AAGAAGAAAAGGAACUGAGA | 85.2296 | 3.6383759 | 49.4382 | 10.739607 |
| SEQ ID NO: 160) | | | | | | | |
| (SEQ ID NO: 305: | 350 | UCUUCUCAGUUCCUUUUCUUCU | AAGAAAAGGAACUGAGAAGA | 95.922733 | 1.8398638 | 48.673467 | 2.4293042 |
| SEQ ID NO: 161) | | | | | | | |
| (SEQ ID NO: 306: | 353 | UGUUCUUCUCAGUUCCUUUUCU | AAAAGGAACUGAGAAGAACA | 94.5707 | 6.3835906 | 41.7594 | 7.1734133 |
| SEQ ID NO: 162) | | | | | | | |

| | | | | | | | |
|---|---|---|---|---|---|---|---|
| (SEQ ID NO: 53: | 358 | UAUGUAGUUCUUCUCAGUUCCU | GAACUGAGAAGAACUACAUA | 64.678 | 1.6125137 | 30.536533 | 2.9373369 |
| SEQ ID NO: 114) | | | | | | | |
| (SEQ ID NO: 307: | 359 | UUAUGUAGUUCUUCUCAGUUCC | AACUGAGAAGAACUACAUAA | 79.186633 | 10.01101 | 47.830533 | 10.387164 |
| SEQ ID NO: 163) | | | | | | | |
| (SEQ ID NO: 54: | 362 | UUUAUAUGUAGUUCUUCUCAGU | UGAGAAGAACUACAUAUAAA | 52.007667 | 5.7221394 | 23.992967 | 3.3882784 |
| SEQ ID NO: 115) | | | | | | | |
| (SEQ ID NO: 308: | 364 | UGUUUAUAUGUAGUUCUUCUCA | AGAAGAACUACAUAUAAACA | 60.327833 | 6.2015425 | 44.081067 | 12.308883 |
| SEQ ID NO: 164) | | | | | | | |
| (SEQ ID NO: 27: | 366 | UUAGUUUAUAUGUAGUUCUUCU | AAGAACUACAUAUAAACUAA | 63.427533 | 2.85008 | 28.985067 | 4.8675553 |
| SEQ ID NO: 88) | | | | | | | |
| (SEQ ID NO: 309: | 369 | UUUGUAGUUUAUAUGUAGUUCU | AACUACAUAUAAACUACAAA | 62.857633 | 7.055527 | 36.931633 | 7.0092183 |
| SEQ ID NO: 165) | | | | | | | |
| (SEQ ID NO: 310: | 375 | UUUUGACUUGUAGUUUAUAUGU | AUAUAAACUACAAGUCAAAA | 62.0823 | 9.0672152 | 50.7904 | 27.221904 |
| SEQ ID NO: 166) | | | | | | | |
| (SEQ ID NO: 311: | 389 | UUUUACCUCUUCAUUUUUGACU | UCAAAAAUGAAGAGGUAAAA | 118.18367 | 1.2517491 | 50.8274 | 3.8309279 |
| SEQ ID NO: 167) | | | | | | | |
| (SEQ ID NO: 55: | 403 | UCAAGUGACAUAUUCUUUACCU | GUAAAGAAUAUGUCACUUGA | 50.322667 | 4.8073004 | 23.886167 | 3.1825861 |
| SEQ ID NO: 116) | | | | | | | |
| (SEQ ID NO: 56: | 415 | UUUGAGUUGAGUUCAAGUGACA | UCACUUGAACUCAACUCAAA | 69.345933 | 15.4121 | 31.234733 | 1.8142841 |
| SEQ ID NO: 117) | | | | | | | |
| (SEQ ID NO: 312: | 417 | UUUUUGAGUUGAGUUCAAGUGA | ACUUGAACUCAACUCAAAAA | 80.624467 | 7.0841712 | 101.0632 | 8.8073837 |
| SEQ ID NO: 168) | | | | | | | |
| (SEQ ID NO: 313: | 425 | UCUUUCAAGUUUUGAGUUGAGU | UCAACUCAAAACUUGAAAGA | 82.508533 | 5.3567098 | 55.001833 | 3.5084995 |
| SEQ ID NO: 169) | | | | | | | |
| (SEQ ID NO: 314: | 440 | UUUUUCUUCUAGGAGGCUUUCA | AAAGCCUCCUAGAAGAAAAA | 87.816433 | 2.6304888 | 53.872633 | 11.02833 |
| SEQ ID NO: 170) | | | | | | | |
| (SEQ ID NO: 315: | 443 | UAUUUUUUCUUCUAGGAGGCUU | GCCUCCUAGAAGAAAAAAUA | 57.2616 | 4.95955 | 35.023633 | 3.3959716 |
| SEQ ID NO: 171) | | | | | | | |
| (SEQ ID NO: 316: | 444 | UAAUUUUUUCUUCUAGGAGGCU | CCUCCUAGAAGAAAAAAUUA | 55.3547 | 7.9583484 | 33.086767 | 0.842275 |
| SEQ ID NO: 172) | | | | | | | |
| (SEQ ID NO: 317: | 445 | UGAAUUUUUUCUUCUAGGAGGC | CUCCUAGAAGAAAAAAUUCA | 65.170533 | 9.0766225 | 50.532433 | 19.274533 |
| SEQ ID NO: 173) | | | | | | | |
| (SEQ ID NO: 318: | 448 | UGUAGAAUUUUUUCUUCUAGGA | CUAGAAGAAAAAAUUCUACA | 61.441067 | 2.2395317 | 40.896667 | 5.4915157 |
| SEQ ID NO: 174) | | | | | | | |
| (SEQ ID NO: 319: | 467 | UUAUUUCACUUUUUGUUGAAGU | UUCAACAAAAAGUGAAAUAA | 53.604333 | 5.6668526 | 35.461133 | 5.5902173 |
| SEQ ID NO: 175) | | | | | | | |
| (SEQ ID NO: 320: | 470 | UAAAUAUUUCACUUUUUGUUGA | AACAAAAAGUGAAAUAUUUA | 79.7731 | 11.290485 | 35.922333 | 11.652346 |
| SEQ ID NO: 176) | | | | | | | |
| (SEQ ID NO: 321: | 471 | UUAAAUAUUUCACUUUUUGUUG | ACAAAAAGUGAAAUAUUUAA | 84.426767 | 16.359293 | 50.253133 | 4.1183419 |
| SEQ ID NO: 177) | | | | | | | |
| (SEQ ID NO: 322: | 472 | UCUAAAUAUUUCACUUUUUGUU | CAAAAAGUGAAAUAUUUAGA | 65.2547 | 3.9430278 | 47.642033 | 5.9982093 |
| SEQ ID NO: 178) | | | | | | | |
| (SEQ ID NO: 323: | 481 | UGUUGCUCUUCUAAAUAUUUCA | AAAUAUUUAGAAGAGCAACA | 95.753833 | 6.3840088 | 84.063433 | 2.9916751 |
| SEQ ID NO: 179) | | | | | | | |
| (SEQ ID NO: 57: | 491 | UAAGUUAGUUAGUUGCUCUUCU | AAGAGCAACUAACUAACUUA | 33.247167 | 4.7473982 | 17.260267 | 2.9985122 |
| SEQ ID NO: 118) | | | | | | | |
| (SEQ ID NO: 30: | 493 | UUUAAGUUAGUUAGUUGCUCUU | GAGCAACUAACUAACUUAAA | 60.394867 | 8.550497 | 30.0198 | 2.6377085 |
| SEQ ID NO: 91) | | | | | | | |
| (SEQ ID NO: 31: | 494 | UAUUAAGUUAGUUAGUUGCUCU | AGCAACUAACUAACUUAAUA | 31.838033 | 2.5026453 | 16.757633 | 1.0421251 |
| SEQ ID NO: 92) | | | | | | | |
| (SEQ ID NO: 58: | 496 | UGAAUUAAGUUAGUUAGUUGCU | CAACUAACUAACUUAAUUCA | 65.6962 | 2.3828708 | 25.028133 | 2.342104 |
| SEQ ID NO: 119) | | | | | | | |
| (SEQ ID NO: 324: | 500 | UUUUUGAAUUAAGUUAGUUAGU | UAACUAACUUAAUUCAAAAA | 97.3836 | 7.0199213 | 46.2078 | 8.8036166 |
| SEQ ID NO: 180) | | | | | | | |
| (SEQ ID NO: 32: | 504 | UUUGAUUUUGAAUUAAGUUAGU | UAACUUAAUUCAAAAUCAAA | 77.550233 | 3.610039 | 27.3279 | 3.1461421 |
| SEQ ID NO: 93) | | | | | | | |
| (SEQ ID NO: 6: | 543 | UUUUAAGUGAAGUUACUUCUGG | AGAAGUAACUUCACUUAAAA | 45.7161 | 3.488431 | 17.9783 | 1.5298929 |
| SEQ ID NO: 67) | | | | | | | |
| (SEQ ID NO: 35: | 569 | UCUAUUAUCUUGUUUUUCUACA | UAGAAAAACAAGAUAAUAGA | 65.361567 | 2.3323769 | 28.518867 | 3.3703379 |
| SEQ ID NO: 96) | | | | | | | |
| (SEQ ID NO: 325: | 577 | UCUUUGAUGCUAUUAUCUUGUU | CAAGAUAAUAGCAUCAAAGA | 114.21473 | 8.3015935 | 72.113933 | 5.4392455 |
| SEQ ID NO: 181) | | | | | | | |
| (SEQ ID NO: 326: | 588 | UCUGGAGAAGGUCUUUGAUGCU | CAUCAAAGACCUUCUCCAGA | 101.64243 | 4.1554567 | 57.086033 | 1.3809388 |
| SEQ ID NO: 182) | | | | | | | |
| (SEQ ID NO: 327: | 635 | UAUUUGACUAUGCUGUUGGUUU | ACCAACAGCAUAGUCAAAUA | 94.039567 | 4.742873 | 48.877433 | 1.4459471 |
| SEQ ID NO: 183) | | | | | | | |
| (SEQ ID NO: 328: | 637 | UUUAUUUGACUAUGCUGUUGGU | CAACAGCAUAGUCAAAUAAA | 111.3027 | 5.3796013 | 78.620533 | 12.135312 |
| SEQ ID NO: 184) | | | | | | | |
| (SEQ ID NO: 62: | 638 | UUUUAUUUGACUAUGCUGUUGG | AACAGCAUAGUCAAAUAAAA | 62.323233 | 5.6744147 | 22.150367 | 3.2152643 |
| SEQ ID NO: 123) | | | | | | | |
| (SEQ ID NO: 329: | 640 | UCUUUUAUUUGACUAUGCUGUU | CAGCAUAGUCAAAUAAAAGA | 90.1718 | 1.3542803 | 42.463733 | 6.6050357 |
| SEQ ID NO: 185) | | | | | | | |
| (SEQ ID NO: 330: | 643 | UUUUCUUUUAUUUGACUAUGCU | CAUAGUCAAAUAAAAGAAAA | 76.853333 | 3.3724043 | 37.182733 | 5.3393213 |
| SEQ ID NO: 186) | | | | | | | |
| (SEQ ID NO: 331: | 644 | UAUUUCUUUUAUUUGACUAUGC | AUAGUCAAAUAAAAGAAAUA | 137.05967 | 9.7924733 | 42.786633 | 5.1706423 |
| SEQ ID NO: 187) | | | | | | | |
| (SEQ ID NO: 332: | 648 | UUUCUAUUUCUUUUAUUUGACU | UCAAAUAAAAGAAAUAGAAA | 140.03297 | 9.4930861 | 73.5336 | 15.295446 |
| SEQ ID NO: 188) | | | | | | | |
| (SEQ ID NO: 333: | 650 | UUUUUCUAUUUCUUUUAUUUGA | AAAUAAAAGAAAUAGAAAAA | 137.60857 | 18.117029 | 69.188333 | 13.17626 |
| SEQ ID NO: 189) | | | | | | | |
| (SEQ ID NO: 334: | 692 | UGAAAUUUCUGUGGGUUCUUGA | AAGAACCCACAGAAAUUUCA | 92.6653 | 9.4770748 | 41.4271 | 5.4535003 |
| SEQ ID NO: 190) | | | | | | | |
| (SEQ ID NO: 335: | 698 | UGAUAGAGAAAUUUCUGUGGGU | CCACAGAAAUUUCUCUAUCA | 83.653067 | 2.7519067 | 45.535367 | 6.702345 |
| SEQ ID NO: 191) | | | | | | | |
| (SEQ ID NO: 37: | 700 | UAAGAUAGAGAAAUUUCUGUGG | ACAGAAAUUUCUCUAUCUUA | 68.411667 | 0.8878605 | 28.9777 | 1.7445077 |
| SEQ ID NO: 98) | | | | | | | |
| (SEQ ID NO: 336: | 721 | UUUCUUGGUGCUCUUGGCUUGG | AAGCCAAGAGCACCAAGAAA | 152.11207 | 9.2513988 | 111.08723 | 51.913069 |
| SEQ ID NO: 192) | | | | | | | |
| (SEQ ID NO: 337: | 724 | UUAGUUCUUGGUGCUCUUGGCU | CCAAGAGCACCAAGAACUAA | 82.261967 | 4.9682566 | 42.907967 | 5.7810435 |
| SEQ ID NO: 193) | | | | | | | |
| (SEQ ID NO: 338: | 725 | UGUAGUUCUUGGUGCUCUUGGC | CAAGAGCACCAAGAACUACA | 98.714333 | 3.1006466 | 54.5726 | 10.375489 |
| SEQ ID NO: 194) | | | | | | | |
| (SEQ ID NO: 339: | 735 | UAAGAAAGGGAGUAGUUCUUGG | AAGAACUACUCCCUUUCUUA | 88.692933 | 1.5607667 | 43.971367 | 6.8420604 |
| SEQ ID NO: 195) | | | | | | | |
| (SEQ ID NO: 39: | 738 | UCUGAAGAAAGGGAGUAGUUCU | AACUACUCCCUUUCUUCAGA | 68.641367 | 5.654269 | 28.614433 | 4.9746183 |
| SEQ ID NO: 100) | | | | | | | |
| (SEQ ID NO: 340: | 760 | UUUACAUUUCUUAUUUCAUUCA | AAUGAAAUAAGAAAUGUAAA | 73.978633 | 2.8610971 | 45.126433 | 3.4099763 |
| SEQ ID NO: 196) | | | | | | | |
| (SEQ ID NO: 341: | 848 | UGAGUUGCUGGGUCUGAUGGCA | CCAUCAGACCCAGCAACUCA | 99.093567 | 2.4467283 | 57.508933 | 7.4624154 |
| SEQ ID NO: 197) | | | | | | | |
| (SEQ ID NO: 342: | 853 | UCUUGAGAGUUGCUGGGUCUGA | AGACCCAGCAACUCUCAAGA | 91.759 | 3.4622054 | 53.417167 | 8.0953572 |
| SEQ ID NO: 198) | | | | | | | |
| (SEQ ID NO: 343: | 855 | UAACUUGAGAGUUGCUGGGUCU | ACCCAGCAACUCUCAAGUUA | 95.9127 | 5.4609295 | 59.1318 | 10.959826 |
| SEQ ID NO: 199) | | | | | | | |
| (SEQ ID NO: 40: | 857 | UAAAACUUGAGAGUUGCUGGGU | CCAGCAACUCUCAAGUUUUA | 59.400233 | 2.7747281 | 24.045367 | 4.229129 |
| SEQ ID NO: 101) | | | | | | | |
| (SEQ ID NO: 344: | 858 | UAAAAACUUGAGAGUUGCUGGG | CAGCAACUCUCAAGUUUUUA | 76.7912 | 4.5216948 | 45.182267 | 8.819548 |
| SEQ ID NO: 634) | | | | | | | |
| (SEQ ID NO: 9: | 859 | UGAAAAACUUGAGAGUUGCUGG | AGCAACUCUCAAGUUUUUCA | 57.659033 | 4.7496144 | 23.821 | 1.6344765 |
| SEQ ID NO: 70) | | | | | | | |
| (SEQ ID NO: 345: | 861 | UAUGAAAAACUUGAGAGUUGCU | CAACUCUCAAGUUUUUCAUA | 71.069233 | 5.2775892 | 58.444467 | 20.132003 |
| SEQ ID NO: 201) | | | | | | | |
| (SEQ ID NO: 346: | 867 | UGUAGACAUGAAAAACUUGAGA | UCAAGUUUUUCAUGUCUACA | 81.9428 | 5.4334955 | 45.0543 | 3.7082459 |
| SEQ ID NO: 202) | | | | | | | |
| (SEQ ID NO: 347: | 900 | UUAAUGUCCAUGGACUACCUGA | AGGUAGUCCAUGGACAUUAA | 67.274567 | 4.2862834 | 31.388467 | 2.1035597 |
| SEQ ID NO: 203) | | | | | | | |
| (SEQ ID NO: 348: | 902 | UAUUAAUGUCCAUGGACUACCU | GUAGUCCAUGGACAUUAAUA | 108.7726 | 7.9744193 | 84.910067 | 8.4569033 |
| SEQ ID NO: 204) | | | | | | | |
| (SEQ ID NO: 349: | 903 | UAAUUAAUGUCCAUGGACUACC | UAGUCCAUGGACAUUAAUUA | 77.622833 | 2.5979571 | 36.826867 | 4.445192 |
| SEQ ID NO: 205) | | | | | | | |
| (SEQ ID NO: 41: | 906 | UUUGAAUUAAUGUCCAUGGACU | UCCAUGGACAUUAAUUCAAA | 71.157233 | 3.7079924 | 36.405733 | 0.8768475 |
| SEQ ID NO: 102) | | | | | | | |
| (SEQ ID NO: 350: | 909 | UAUGUUGAAUUAAUGUCCAUGG | AUGGACAUUAAUUCAACAUA | 113.2428 | 3.3691547 | 81.756033 | 19.427249 |
| SEQ ID NO: 206) | | | | | | | |
| (SEQ ID NO: 351: | 915 | UUAUUCGAUGUUGAAUUAAUGU | AUUAAUUCAACAUCGAAUAA | 132.1274 | 21.339567 | 58.182467 | 10.000921 |
| SEQ ID NO: 207) | | | | | | | |
| (SEQ ID NO: 352: | 929 | UUUUUGUGAUCCAUCUAUUCGA | GAAUAGAUGGAUCACAAAAA | 185.28237 | 28.947359 | 93.806867 | 15.706993 |
| SEQ ID NO: 208) | | | | | | | |
| (SEQ ID NO: 353: | 930 | UGUUUUGUGAUCCAUCUAUUCG | AAUAGAUGGAUCACAAAACA | 143.0759 | 7.9791352 | 61.372733 | 15.539965 |
| SEQ ID NO: 209) | | | | | | | |
| (SEQ ID NO: 354: | 935 | UUUGAAGUUUUGUGAUCCAUCU | AUGGAUCACAAAACUUCAAA | 129.94767 | 13.627957 | 70.935 | 8.4723531 |
| SEQ ID NO: 210) | | | | | | | |
| (SEQ ID NO: 355: | 956 | UUUGUAGUUCUCCCACGUUUCA | AAACGUGGGAGAACUACAAA | 148.64763 | 13.5027 | 110.03653 | 11.744245 |
| SEQ ID NO: 211) | | | | | | | |
| (SEQ ID NO: 356: | 960 | UAUAUUUGUAGUUCUCCCACGU | GUGGGAGAACUACAAAUAUA | 132.17717 | 12.151453 | 104.19597 | 29.315333 |
| SEQ ID NO: 212) | | | | | | | |
| (SEQ ID NO: 357: | 961 | UCAUAUUUGUAGUUCUCCCACG | UGGGAGAACUACAAAUAUGA | 103.725 | 7.7607682 | 47.876967 | 6.2847505 |
| SEQ ID NO: 213) | | | | | | | |
| (SEQ ID NO: 358: | 964 | UAACCAUAUUUGUAGUUCUCCC | GAGAACUACAAAUAUGGUUA | 118.90777 | 10.384789 | 57.7426 | 6.7707745 |
| SEQ ID NO: 214) | | | | | | | |
| (SEQ ID NO: 42: | 965 | UAAACCAUAUUUGUAGUUCUCC | AGAACUACAAAUAUGGUUUA | 82.127033 | 9.8030209 | 27.6747 | 3.8724921 |
| SEQ ID NO: 103) | | | | | | | |
| (SEQ ID NO: 359: | 1008 | UUAUCUUCUCUAGGCCCAACCA | GUUGGGCCUAGAGAAGAUAA | 141.8275 | 16.388851 | 96.864367 | 15.759358 |
| SEQ ID NO: 215) | | | | | | | |
| (SEQ ID NO: 360: | 1009 | UAUAUCUUCUCUAGGCCCAACC | UUGGGCCUAGAGAAGAUAUA | 125.25507 | 7.9341453 | 81.532133 | 3.2216714 |
| SEQ ID NO: 216) | | | | | | | |
| (SEQ ID NO: 361: | 1013 | UGAGUAUAUCUUCUCUAGGCCC | GCCUAGAGAAGAUAUACUCA | 113.2969 | 6.2405098 | 69.3611 | 18.425281 |
| SEQ ID NO: 217) | | | | | | | |
| (SEQ ID NO: 362: | 1030 | UUAGAUUGCUUCACUAUGGAGU | UCCAUAGUGAAGCAAUCUAA | 101.4948 | 5.4443606 | 47.836067 | 8.3064239 |
| SEQ ID NO: 218) | | | | | | | |
| (SEQ ID NO: 363: | 1032 | UAUUAGAUUGCUUCACUAUGGA | CAUAGUGAAGCAAUCUAAUA | 94.281133 | 9.8842229 | 43.5399 | 10.690291 |
| SEQ ID NO: 219) | | | | | | | |
| (SEQ ID NO: 364: | 1033 | UAAUUAGAUUGCUUCACUAUGG | AUAGUGAAGCAAUCUAAUUA | 80.2373 | 8.3302341 | 30.678633 | 5.0638987 |
| SEQ ID NO: 220) | | | | | | | |
| (SEQ ID NO: 365: | 1061 | UCAGUCUUCCAACUCAAUUCGU | GAAUUGAGUUGGAAGACUGA | 109.6143 | 7.1361751 | 66.678467 | 6.7831127 |
| SEQ ID NO: 221) | | | | | | | |
| (SEQ ID NO: 366: | 1078 | UAAUGUUUGUUGUCUUUCCAGU | UGGAAAGACAACAAACAUUA | 102.99897 | 7.7998012 | 55.368367 | 3.3318317 |
| SEQ ID NO: 222) | | | | | | | |
| (SEQ ID NO: 367: | 1080 | UAUAAUGUUUGUUGUCUUUCCA | GAAAGACAACAAACAUUAUA | 103.75123 | 11.036301 | 74.157033 | 12.440365 |
| SEQ ID NO: 223) | | | | | | | |
| (SEQ ID NO: 44: | 1081 | UUAUAAUGUUUGUUGUCUUUCC | AAAGACAACAAACAUUAUAA | 72.085467 | 6.0336195 | 28.3018 | 2.9335243 |
| SEQ ID NO: 105) | | | | | | | |
| (SEQ ID NO: 368: | 1084 | UCAAUAUAAUGUUUGUUGUCUU | GACAACAAACAUUAUAUUGA | 109.78223 | 8.1878628 | 77.8338 | 8.5819048 |
| SEQ ID NO: 224) | | | | | | | |
| (SEQ ID NO: 369: | 1087 | UAUUCAAUAUAAUGUUUGUUGU | AACAAACAUUAUAUUGAAUA | 126.2894 | 7.5927047 | 94.415 | 12.560197 |
| SEQ ID NO: 225) | | | | | | | |
| (SEQ ID NO: 370: | 1090 | UAAUAUUCAAUAUAAUGUUUGU | AAACAUUAUAUUGAAUAUUA | 89.525567 | 4.8574535 | 43.538433 | 11.119729 |
| SEQ ID NO: 226) | | | | | | | |
| (SEQ ID NO: 371: | 1121 | UUAGUUGGUUUCGUGAUUUCCC | GAAAUCACGAAACCAACUAA | 70.1635 | 3.554717 | 35.036967 | 5.1468698 |
| SEQ ID NO: 227) | | | | | | | |
| (SEQ ID NO: 372: | 1122 | UAUAGUUGGUUUCGUGAUUUCC | AAAUCACGAAACCAACUAUA | 83.583633 | 7.1836706 | 57.0473 | 7.5819033 |
| SEQ ID NO: 228) | | | | | | | |
| (SEQ ID NO: 373: | 1129 | UGUAGCGUAUAGUUGGUUUCGU | GAAACCAACUAUACGCUACA | 88.864933 | 7.9989439 | 46.698667 | 1.0430542 |
| SEQ ID NO: 229) | | | | | | | |
| (SEQ ID NO: 374: | 1166 | UAUUGCAUUGGGGACAUUGCCA | GCAAUGUCCCCAAUGCAAUA | 88.390133 | 10.063767 | 46.541267 | 3.1804305 |
| SEQ ID NO: 230) | | | | | | | |
| (SEQ ID NO: 375: | 1167 | UGAUUGCAUUGGGGACAUUGCC | CAAUGUCCCCAAUGCAAUCA | 115.077 | 3.3146796 | 111.374 | 18.734875 |
| SEQ ID NO: 231) | | | | | | | |
| (SEQ ID NO: 376: | 1176 | UGUUUUCCGGGAUUGCAUUGGG | CAAUGCAAUCCCGGAAAACA | 94.2486 | 11.377036 | 81.139 | 5.0015777 |
| SEQ ID NO: 232) | | | | | | | |
| (SEQ ID NO: 377: | 1182 | UAUCUUUGUUUUCCGGGAUUGC | AAUCCCGGAAAACAAAGAUA | 97.6698 | 11.547892 | 109.07143 | 54.790767 |
| SEQ ID NO: 233) | | | | | | | |
| (SEQ ID NO: 378: | 1188 | UCACCAAAUCUUUGUUUUCCGG | GGAAAACAAAGAUUUGGUGA | 52.370733 | 11.892447 | 123.1813 | 87.066701 |
| SEQ ID NO: 234) | | | | | | | |
| (SEQ ID NO: 379: | 1195 | UUAGAAAACACCAAAUCUUUGU | AAAGAUUUGGUGUUUUCUAA | 77.0324 | 1.5308621 | 43.206133 | 9.2457205 |
| SEQ ID NO: 235) | | | | | | | |
| (SEQ ID NO: 380: | 1204 | UGAUCCCAAGUAGAAAACACCA | GUGUUUUCUACUUGGGAUCA | 103.65283 | 8.106543 | 129.6379 | 40.457466 |
| SEQ ID NO: 236) | | | | | | | |
| (SEQ ID NO: 381: | 1215 | UUUUUGCUUUGUGAUCCCAAGU | UUGGGAUCACAAAGCAAAAA | 98.8224 | 4.3587929 | 76.0444 | 11.570459 |
| SEQ ID NO: 237) | | | | | | | |
| (SEQ ID NO: 382: | 1219 | UGUCCUUUUGCUUUGUGAUCCC | GAUCACAAAGCAAAAGGACA | 104.02257 | 6.1514778 | 78.301967 | 9.1899298 |
| SEQ ID NO: 238) | | | | | | | |
| (SEQ ID NO: 383: | 1269 | UCUCAUCAUGCCACCACCAGCC | CUGGUGGUGGCAUGAUGAGA | 103.70937 | 8.1498855 | 126.52803 | 42.205361 |
| SEQ ID NO: 239) | | | | | | | |
| (SEQ ID NO: 384: | 1273 | UCACACUCAUCAUGCCACCACC | UGGUGGCAUGAUGAGUGUGA | 107.83373 | 5.7365498 | 99.9397 | 1.3136097 |
| SEQ ID NO: 240) | | | | | | | |
| (SEQ ID NO: 385: | 1279 | UUUUCUCCACACUCAUCAUGCC | CAUGAUGAGUGUGGAGAAAA | 121.94393 | 15.810038 | 70.0712 | 29.903818 |
| SEQ ID NO: 241) | | | | | | | |
| (SEQ ID NO: 386: | 1280 | UUUUUCUCCACACUCAUCAUGC | AUGAUGAGUGUGGAGAAAAA | 87.6833 | 9.0146735 | 43.7414 | 18.931566 |
| SEQ ID NO: 242) | | | | | | | |
| (SEQ ID NO: 387: | 1288 | UUUAGGUUGUUUUCUCCACACU | UGUGGAGAAAACAACCUAAA | 22.05941 | 90.047779 | 65.621033 | 23.955834 |
| SEQ ID NO: 243) | | | | | | | |
| (SEQ ID NO: 388: | 1290 | UAUUUAGGUUGUUUUCUCCACA | UGGAGAAAACAACCUAAAUA | 76.546567 | 1.4885844 | 51.554 | 4.774882 |
| SEQ ID NO: 244) | | | | | | | |
| (SEQ ID NO: 389: | 1300 | UUAUAUUUACCAUUUAGGUUGU | AACCUAAAUGGUAAAUAUAA | 85.734367 | 4.1702586 | 62.483033 | 4.8870038 |
| SEQ ID NO: 245) | | | | | | | |
| (SEQ ID NO: 390: | 1301 | UUUAUAUUUACCAUUUAGGUUG | ACCUAAAUGGUAAAUAUAAA | 68.444567 | 7.471757 | 41.968867 | 5.2868984 |
| SEQ ID NO: 246) | | | | | | | |
| (SEQ ID NO: 391: | 1302 | UGUUAUAUUUACCAUUUAGGUU | CCUAAAUGGUAAAUAUAACA | 84.578233 | 0.4915932 | 40.932367 | 18.304362 |
| SEQ ID NO: 247) | | | | | | | |
| (SEQ ID NO: 392: | 1304 | UUUGUUAUAUUUACCAUUUAGG | UAAAUGGUAAAUAUAACAAA | 75.169933 | 0.3859007 | 44.0614 | 2.9868971 |
| SEQ ID NO: 248) | | | | | | | |
| (SEQ ID NO: 393: | 1310 | UCUUGGUUUGUUAUAUUUACCA | GUAAAUAUAACAAACCAAGA | 80.955133 | 4.0044948 | 52.750567 | 5.9144115 |
| SEQ ID NO: 249) | | | | | | | |
| (SEQ ID NO: 394: | 1372 | UAUAACCUUCCAUUUUGAGACU | UCUCAAAAUGGAAGGUUAUA | 82.279633 | 4.4737465 | 56.732833 | 5.0342632 |
| SEQ ID NO: 250) | | | | | | | |
| (SEQ ID NO: 11: | 1374 | UGUAUAACCUUCCAUUUUGAGA | UCAAAAUGGAAGGUUAUACA | 47.419233 | 1.5257463 | 20.3444 | 1.6642044 |
| SEQ ID NO: 72) | | | | | | | |
| (SEQ ID NO: 12: | 2382 | UUUUAUAGAGUAUAACCUUCCA | GAAGGUUAUACUCUAUAAAA | 52.965733 | 5.9697325 | 25.163667 | 4.6561875 |
| SEQ ID NO: 73) | | | | | | | |
| (SEQ ID NO: 395: | 1383 | UUUUUAUAGAGUAUAACCUUCC | AAGGUUAUACUCUAUAAAAA | 84.7974 | 8.9870304 | 40.742033 | 10.249496 |
| SEQ ID NO: 251) | | | | | | | |
| (SEQ ID NO: 396: | 1385 | UGAUUUUAUAGAGUAUAACCUU | GGUUAUACUCUAUAAAAUCA | 90.5518 | 5.754896 | 47.735167 | 14.146511 |
| SEQ ID NO: 252) | | | | | | | |
| (SEQ ID NO: 13: | 2387 | UUUGAUUUUAUAGAGUAUAACC | UUAUACUCUAUAAAAUCAAA | 49.542867 | 5.4764675 | 20.700367 | 3.9959345 |
| SEQ ID NO: 74) | | | | | | | |
| (SEQ ID NO: 397: | 1393 | UUUUUGGUUGAUUUUAUAGAGU | UCUAUAAAAUCAACCAAAAA | 92.809833 | 6.3035405 | 50.460833 | 5.3540639 |
| SEQ ID NO: 253) | | | | | | | |
| (SEQ ID NO: 398: | 1395 | UCAUUUUGGUUGAUUUUAUAGA | UAUAAAAUCAACCAAAAUGA | 64.130467 | 2.0472791 | 52.6753 | 13.564506 |
| SEQ ID NO: 254) | | | | | | | |
| (SEQ ID NO: 399: | 1404 | UAUGGAUCAACAUUUUGGUUGA | AACCAAAAUGUUGAUCCAUA | 173.1365 | 29.476142 | 70.1206 | 23.099461 |
| SEQ ID NO: 255) | | | | | | | |
| (SEQ ID NO: 400: | 1417 | UCUGAAUCUGUUGGAUGGAUCA | AUCCAUCCAACAGAUUCAGA | 87.26 | 6.6062014 | 71.0928 | 11.558223 |
| SEQ ID NO: 256) | | | | | | | |
| (SEQ ID NO: 401: | 1420 | UUUUCUGAAUCUGUUGGAUGGA | CAUCCAACAGAUUCAGAAAA | 121.9751 | 19.330767 | 106.1185 | 42.438487 |
| SEQ ID NO: 257) | | | | | | | |
| (SEQ ID NO: 402: | 1421 | UCUUUCUGAAUCUGUUGGAUGG | AUCCAACAGAUUCAGAAAGA | 96.614167 | 7.881981 | 63.7182 | 9.1294267 |
| SEQ ID NO: 258) | | | | | | | |
| (SEQ ID NO: 403: | 1424 | UAAGCUUUCUGAAUCUGUUGGA | CAACAGAUUCAGAAAGCUUA | 62.765633 | 5.2521074 | 32.512133 | 5.7431891 |
| SEQ ID NO: 259) | | | | | | | |
| (SEQ ID NO: 200: | 1428 | UUUCAAAGCUUUCUGAAUCUGU | AGAUUCAGAAAGCUUUGAAA | 50.476333 | 1.9068653 | 24.403067 | 2.9538761 |
| SEQ ID NO: 235) | | | | | | | |
| (SEQ ID NO: 404: | 1430 | UCAUUCAAAGCUUUCUGAAUCU | AUUCAGAAAGCUUUGAAUGA | 77.107233 | 4.6970322 | 39.7077 | 6.0068176 |
| SEQ ID NO: 260) | | | | | | | |
| (SEQ ID NO: 405: | 1445 | UAAAUUUGCCUCAGUUCAUUCA | AAUGAACUGAGGCAAAUUUA | 96.511567 | 0.5075969 | 64.195267 | 5.3993091 |
| SEQ ID NO: 261) | | | | | | | |
| (SEQ ID NO: 406: | 1449 | UUUUUAAAUUUGCCUCAGUUCA | AACUGAGGCAAAUUUAAAAA | 92.577967 | 2.5478732 | 64.621 | 7.5239279 |
| SEQ ID NO: 262) | | | | | | | |
| (SEQ ID NO: 407: | 1450 | UCUUUUAAAUUUGCCUCAGUUC | ACUGAGGCAAAUUUAAAAGA | 107.32393 | 7.7834844 | 66.103033 | 2.0528362 |
| SEQ ID NO: 263) | | | | | | | |
| (SEQ ID NO: 408: | 1454 | UUUGCCUUUUAAAUUUGCCUCA | AGGCAAAUUUAAAAGGCAAA | 94.013167 | 15.980062 | 48.363467 | 5.8449752 |
| SEQ ID NO: 264) | | | | | | | |
| (SEQ ID NO: 409: | 1456 | UUAUUGCCUUUUAAAUUUGCCU | GCAAAUUUAAAAGGCAAUAA | 84.605 | 5.7726317 | 56.793433 | 19.518118 |
| SEQ ID NO: 265) | | | | | | | |
| (SEQ ID NO: 410: | 1457 | UUUAUUGCCUUUUAAAUUUGCC | CAAAUUUAAAAGGCAAUAAA | 74.781467 | 2.3691634 | 32.703633 | 4.5081013 |
| SEQ ID NO: 266) | | | | | | | |
| (SEQ ID NO: 411: | 1522 | UUUCUCUUAAGGAUUUAAUACC | UAUUAAAUCCUUAAGAGAAA | 84.0109 | 3.005092 | 35.619533 | 2.3364351 |
| SEQ ID NO: 267) | | | | | | | |

Set 2

| | | | | | | | |
|---|---|---|---|---|---|---|---|
| (SEQ ID NO: 523: | 33 | UCAAUUUCAAGCAACGUGGAAC | UCCACGUUGCUUGAAAUUGA | 83.566667 | 1.2128937 | 59 | 8 |
| SEQ ID NO: 412) | | | | | | | |
| (SEQ ID NO: 524: | 37 | UUUUUCAAUUUCAAGCAACGUG | CGUUGCUUGAAAUUGAAAAA | 68.7 | 2.2501852 | 29.6 | 2.6102363 |
| SEQ ID NO: 413) | | | | | | | |
| (SEQ ID NO: 525: | 62 | UUUAAUUGUGAACAUUUUUAUC | UAAAAAUGUUCACAAUUAAA | 74.233333 | 1.6796164 | 27.833333 | 2.338328 |
| SEQ ID NO: 414) | | | | | | | |
| (SEQ ID NO: 526: | 63 | UCUUAAUUGUGAACAUUUUUAU | AAAAAUGUUCACAAUUAAGA | 97.033333 | 9.2405507 | 53.966667 | 3.0140412 |
| SEQ ID NO: 415) | | | | | | | |
| (SEQ ID NO: 15: | 77 | UAUAAAAAGAAGGAGCUUAAUU | UUAAGCUCCUUCUUUUUAUA | 47.166667 | 3.7100464 | 16.7 | 2.007486 |
| SEQ ID NO: 76) | | | | | | | |
| (SEQ ID NO: 16: | 78 | UAAUAAAAAGAAGGAGCUUAAU | UAAGCUCCUUCUUUUUAUUA | 77.733333 | 4.684134 | 26.866667 | 2.3835082 |
| SEQ ID NO: 77) | | | | | | | |
| (SEQ ID NO: 2: | 79 | UCAAUAAAAAGAAGGAGCUUAA | AAGCUCCUUCUUUUUAUUGA | 48.766667 | 1.034945 | 20.866667 | 0.5044249 |
| SEQ ID NO: 63) | | | | | | | |
| (SEQ ID NO: 17: | 94 | UAAAUAACUAGAGGAACAAUAA | AUUGUUCCUCUAGUUAUUUA | 50.466667 | 6.920822 | 17.166667 | 0.7446103 |
| SEQ ID NO: 78) | | | | | | | |
| (SEQ ID NO: 527: | 98 | UGAGGAAAUAACUAGAGGAACA | UUCCUCUAGUUAUUUCCUCA | 71.833333 | 2.6491089 | 37.1 | 1.3453624 |
| SEQ ID NO: 416) | | | | | | | |
| (SEQ ID NO: 20: | 154 | UUUGAUUUUGGCUCUGGAGAUA | UCUCCAGAGCCAAAAUCAAA | 62.866667 | 8.5159328 | 21.933333 | 0.664162 |
| SEQ ID NO: 81) | | | | | | | |
| (SEQ ID NO: 528: | 155 | UCUUGAUUUUGGCUCUGGAGAU | CUCCAGAGCCAAAAUCAAGA | 80.966667 | 15.908523 | 37.2 | 7.4002252 |
| SEQ ID NO: 417) | | | | | | | |
| (SEQ ID NO: 50: | 169 | UCUAACAUAGCAAAUCUUGAUU | UCAAGAUUUGCUAUGUUAGA | 63.7 | 11.475191 | 22.233333 | 2.2578259 |
| SEQ ID NO: 111) | | | | | | | |
| (SEQ ID NO: 21: | 174 | UAUCGUCUAACAUAGCAAAUCU | AUUUGCUAUGUUAGACGAUA | 54.666667 | 8.5623854 | 22.166667 | 1.3928409 |
| SEQ ID NO: 82) | | | | | | | |
| (SEQ ID NO: 529: | 181 | UUUUUUACAUCGUCUAACAUAG | AUGUUAGACGAUGUAAAAAA | 67.566667 | 8.2795598 | 30.266667 | 2.2578259 |
| SEQ ID NO: 418) | | | | | | | |
| (SEQ ID NO: 22: | 182 | UAUUUUUACAUCGUCUAACAUA | UGUUAGACGAUGUAAAAAUA | 46.333333 | 3.5783298 | 13.466667 | 0.5607535 |
| SEQ ID NO: 83) | | | | | | | |
| (SEQ ID NO: 23: | 185 | UAAAAUUUUUACAUCGUCUAAC | UAGACGAUGUAAAAAUUUUA | 40.5 | 3.356089 | 13.733333 | 1.4518188 |
| SEQ ID NO: 84) | | | | | | | |
| (SEQ ID NO: 530: | 186 | UUAAAAUUUUUACAUCGUCUAA | AGACGAUGUAAAAAUUUUAA | 46.1 | 3.7233497 | 27.3 | 0.305505 |
| SEQ ID NO: 419) | | | | | | | |
| (SEQ ID NO: 531: | 225 | UGUCUUUAAGACCAUGUCCCAA | GGGACAUGGUCUUAAAGACA | 74.133333 | 6.2135157 | 70.433333 | 8.0470146 |
| SEQ ID NO: 420) | | | | | | | |
| (SEQ ID NO: 532: | 226 | UAGUCUUUAAGACCAUGUCCCA | GGACAUGGUCUUAAAGACUA | 128.2 | 6.7039789 | 88.866667 | 2.3103631 |
| SEQ ID NO: 421) | | | | | | | |
| (SEQ ID NO: 533: | 229 | UCAAAGUCUUUAAGACCAUGUC | CAUGGUCUUAAAGACUUUGA | 156.33333 | 15.975015 | 109.56667 | 15.163809 |
| SEQ ID NO: 422) | | | | | | | |
| (SEQ ID NO: 534: | 239 | UGUCUUAUGGACAAAGUCUUUA | AAGACUUUGUCCAUAAGACA | 134.16667 | 8.6971898 | 109.2 | 10.700156 |
| SEQ ID NO: 423) | | | | | | | |
| (SEQ ID NO: 51: | 267 | UUUGAAAUAUGUCAUUAAUUUG | AAUUAAUGACAUAUUUCAAA | 63.7 | 3.5341194 | 23.233333 | 3.2844753 |
| SEQ ID NO: 112) | | | | | | | |
| (SEQ ID NO: 535: | 270 | UUUUUUGAAAUAUGUCAUUAAU | UAAUGACAUAUUUCAAAAAA | 130.53333 | 17.288757 | 64.633333 | 7.4601906 |
| SEQ ID NO: 424) | | | | | | | |
| (SEQ ID NO: 536: | 271 | UGUUUUUGAAAUAUGUCAUUAA | AAUGACAUAUUUCAAAAACA | 91.733333 | 5.621783 | 36.633333 | 2.9672284 |
| SEQ ID NO: 425) | | | | | | | |
| (SEQ ID NO: 537: | 282 | UAAAUAUGUUGAGUUUUUGAAA | UCAAAAACUCAACAUAUUUA | 77 | 0.305505 | 37.2 | 2.9715316 |
| SEQ ID NO: 426) | | | | | | | |
| (SEQ ID NO: 52: | 283 | UCAAAUAUGUUGAGUUUUUGAA | CAAAAACUCAACAUAUUUGA | 71.3 | 3.8850139 | 26.633333 | 2.6834886 |
| SEQ ID NO: 113) | | | | | | | |
| (SEQ ID NO: 538: | 285 | UAUCAAAUAUGUUGAGUUUUUG | AAAACUCAACAUAUUUGAUA | 88.466667 | 3.8324637 | 35.4 | 0.3511885 |
| SEQ ID NO: 427) | | | | | | | |
| (SEQ ID NO: 25: | 296 | UUAAAAAGACUGAUCAAAUAUG | UAUUUGAUCAGUCUUUUUAA | 56.933333 | 6.7296194 | 22.033333 | 2.1137118 |
| SEQ ID NO: 86) | | | | | | | |
| (SEQ ID NO: 4: | 297 | UAUAAAAAGACUGAUCAAAUAU | AUUUGAUCAGUCUUUUUAUA | 62.366667 | 1.449521 | 25.633333 | 3.6956431 |
| SEQ ID NO: 65) | | | | | | | |
| (SEQ ID NO: 539: | 301 | UGAUCAUAAAAAGACUGAUCAA | GAUCAGUCUUUUUAUGAUCA | 96.533333 | 8.0718303 | 55.4 | 2.007486 |
| SEQ ID NO: 428) | | | | | | | |
| (SEQ ID NO: 26: | 304 | UAUAGAUCAUAAAAAGACUGAU | CAGUCUUUUUAUGAUCUAUA | 52.333333 | 2.5392475 | 22.666667 | 2.2519128 |
| SEQ ID NO: 87) | | | | | | | |
| (SEQ ID NO: 540: | 327 | UUUUGAUUUCACUGGUUUGCAG | GCAAACCAGUGAAAUCAAAA | 101.9 | 9.4959641 | 66.3 | 8.3 |
| SEQ ID NO: 429) | | | | | | | |
| (SEQ ID NO: 541: | 330 | UUUCUUUGAUUUCACUGGUUUG | AACCAGUGAAAUCAAAGAAA | 87.1 | 3.5104606 | 40 | 2.2538855 |
| SEQ ID NO: 430) | | | | | | | |
| (SEQ ID NO: 542: | 337 | UUUUCUUCUUCUUUGAUUUCAC | GAAAUCAAAGAAGAAGAAAA | 119.03333 | 9.7160234 | 37.266667 | 2.9812376 |
| SEQ ID NO: 431) | | | | | | | |
| (SEQ ID NO: 543: | 339 | UCUUUUCUUCUUCUUUGAUUUC | AAUCAAAGAAGAAGAAAAGA | 79.2 | 3.9374272 | 34.333333 | 2.490203 |
| SEQ ID NO: 432) | | | | | | | |
| (SEQ ID NO: 544: | 356 | UGUAGUUCUUCUCAGUUCCUUU | AGGAACUGAGAAGAACUACA | 79.533333 | 3.0112751 | 31.966667 | 2.5692628 |
| SEQ ID NO: 433) | | | | | | | |
| (SEQ ID NO: 545: | 360 | UAUAUGUAGUUCUUCUCAGUUC | ACUGAGAAGAACUACAUAUA | 72.733333 | 7.0390182 | 29.2 | 0.1527525 |
| SEQ ID NO: 434) | | | | | | | |
| (SEQ ID NO: 28: | 363 | UUUUAUAUGUAGUUCUUCUCAG | GAGAAGAACUACAUAUAAAA | 51.733333 | 2.8904056 | 18.6 | 0.7211103 |
| SSEQ ID NO: 89) | | | | | | | |
| (SEQ ID NO: 546: | 370 | UCUUGUAGUUUAUAUGUAGUUC | ACUACAUAUAAACUACAAGA | 87.6 | 7.7105988 | 52.333333 | 2.3680747 |
| SEQ ID NO: 435) | | | | | | | |
| (SEQ ID NO: 547: | 372 | UGACUUGUAGUUUAUAUGUAGU | UACAUAUAAACUACAAGUCA | 84.433333 | 8.2870046 | 44.633333 | 5.2333333 |
| SEQ ID NO: 436) | | | | | | | |
| (SEQ ID NO: 548: | 376 | UUUUUGACUUGUAGUUUAUAUG | UAUAAACUACAAGUCAAAAA | 77.1 | 4.0037482 | 59.466667 | 2.3974524 |
| SEQ ID NO: 437) | | | | | | | |
| (SEQ ID NO: 29: | 378 | UAUUUUUGACUUGUAGUUUAUA | UAAACUACAAGUCAAAAAUA | 48.4 | 2.136196 | 21.833333 | 1.2018504 |
| SEQ ID NO: | | | | | | | |
| SEQ ID NO: 90) | | | | | | | |
| (SEQ ID NO: 549: | 379 | UCAUUUUUGACUUGUAGUUUAU | AAACUACAAGUCAAAAAUGA | 95.466667 | 2.3694819 | 43.8 | 4.7843495 |
| SEQ ID NO: 438) | | | | | | | |
| (SEQ ID NO: 550: | 381 | UUUCAUUUUUGACUUGUAGUUU | ACUACAAGUCAAAAAUGAAA | 82.6 | 4.6479386 | 32.8 | 4.8839874 |
| SEQ ID NO: 439) | | | | | | | |
| (SEQ ID NO: 551: | 393 | UAUUCUUUACCUCUUCAUUUUU | AAAUGAAGAGGUAAAGAAUA | 97.4 | 5.3500779 | 50.866667 | 5.1414438 |
| SEQ ID NO: 440) | | | | | | | |
| (SEQ ID NO: 552: | 396 | UCAUAUUCUUUACCUCUUCAUU | UGAAGAGGUAAAGAAUAUGA | 89.4 | 10.695949 | 62.166667 | 6.1690986 |
| SEQ ID NO: 441) | | | | | | | |
| (SEQ ID NO: 5: | 398 | UGACAUAUUCUUUACCUCUUCA | AAGAGGUAAAGAAUAUGUCA | 57.566667 | 4.9092882 | 20.7 | 1.8520259 |
| SEQ ID NO: 66) | | | | | | | |
| (SEQ ID NO: 553: | 418 | UGUUUUGAGUUGAGUUCAAGUG | CUUGAACUCAACUCAAAACA | 87 | 2.4131584 | 52 | 5.4009258 |
| SEQ ID NO: 442) | | | | | | | |
| (SEQ ID NO: 554: | 438 | UUUCUUCUAGGAGGCUUUCAAG | UGAAAGCCUCCUAGAAGAAA | 96.733333 | 4.7054342 | 53.766667 | 3.4671474 |
| SEQ ID NO: 443) | | | | | | | |
| (SEQ ID NO: 555: | 439 | UUUUCUUCUAGGAGGCUUUCAA | GAAAGCCUCCUAGAAGAAAA | 78.3 | 10.570872 | 44.6 | 5.3153865 |
| SEQ ID NO: 444) | | | | | | | |
| (SEQ ID NO: 556: | 441 | UUUUUUCUUCUAGGAGGCUUUC | AAGCCUCCUAGAAGAAAAAA | 121.76667 | 14.653365 | 85.266667 | 6.4576397 |
| SEQ ID NO: 445) | | | | | | | |
| (SEQ ID NO: 557: | 442 | UUUUUUUCUUCUAGGAGGCUUU | AGCCUCCUAGAAGAAAAAAA | 106.1 | 6.0135957 | 77.6 | 6.3846169 |
| SEQ ID NO: 446) | | | | | | | |
| (SEQ ID NO: 558: | 446 | UAGAAUUUUUUCUUCUAGGAGG | UCCUAGAAGAAAAAAUUCUA | 46.6 | 3.3171273 | 28.766667 | 5.028364 |
| SEQ ID NO: 447) | | | | | | | |
| (SEQ ID NO: 59: | 454 | UGUUGAAGUAGAAUUUUUUCUU | GAAAAAAUUCUACUUCAACA | 65.933333 | 2.2407836 | 23.666667 | 2.8179977 |
| SEQ ID NO: 120) | | | | | | | |
| (SEQ ID NO: 60: | 456 | UUUGUUGAAGUAGAAUUUUUUC | AAAAAUUCUACUUCAACAAA | 56.2 | 7.5883683 | 25.533333 | 2.3596139 |
| SEQ ID NO: 121) | | | | | | | |
| (SEQ ID NO: 559: | 457 | UUUUGUUGAAGUAGAAUUUUUU | AAAAUUCUACUUCAACAAAA | 82.3 | 4.8809152 | 52.966667 | 0.9261629 |
| SEQ ID NO: 448) | | | | | | | |
| (SEQ ID NO: 560: | 458 | UUUUUGUUGAAGUAGAAUUUUU | AAAUUCUACUUCAACAAAAA | 88.933333 | 7.7365654 | 46.433333 | 4.2230847 |
| SEQ ID NO: 449) | | | | | | | |
| (SEQ ID NO: 561: | 459 | UUUUUUGUUGAAGUAGAAUUUU | AAUUCUACUUCAACAAAAAA | 91.6 | 5.0362023 | 61.8 | 4.2394968 |
| SEQ ID NO: 450) | | | | | | | |
| (SEQ ID NO: 562: | 460 | UCUUUUUGUUGAAGUAGAAUUU | AUUCUACUUCAACAAAAAGA | 84.7 | 3.4122329 | 86.4 | 8.1283455 |
| SEQ ID NO: 451) | | | | | | | |
| (SEQ ID NO: 61: | 464 | UUUCACUUUUUGUUGAAGUAGA | UACUUCAACAAAAAGUGAAA | 56.166667 | 2.7846803 | 26.2 | 0.7211103 |
| SEQ ID NO: 122) | | | | | | | |
| (SEQ ID NO: 563: | 466 | UAUUUCACUUUUUGUUGAAGUA | CUUCAACAAAAAGUGAAAUA | 82.2 | 2.1126603 | 70.066667 | 1.5602706 |
| SEQ ID NO: 452) | | | | | | | |
| (SEQ ID NO: 564: | 468 | UAUAUUUCACUUUUUGUUGAAG | UCAACAAAAAGUGAAAUAUA | 74.533333 | 4.5961336 | 38.733333 | 0.8373238 |
| SEQ ID NO: 453) | | | | | | | |
| (SEQ ID NO: 33: | 474 | UUUCUAAAUAUUUCACUUUUUG | AAAAGUGAAAUAUUUAGAAA | 52.933333 | 1.7854349 | 24.066667 | 0.9404491 |
| SEQ ID NO: 94) | | | | | | | |
| (SEQ ID NO: 565: | 475 | UCUUCUAAAUAUUUCACUUUUU | AAAGUGAAAUAUUUAGAAGA | 76 | 2.0207259 | 41.866667 | 1.7265894 |
| SEQ ID NO: 454) | | | | | | | |
| (SEQ ID NO: 34: | 488 | UUUAGUUAGUUGCUCUUCUAAA | UAGAAGAGCAACUAACUAAA | 48.666667 | 1.5070206 | 20.033333 | 0.5238745 |
| SEQ ID NO: 95) | | | | | | | |
| (SEQ ID NO: 566: | 495 | UAAUUAAGUUAGUUAGUUGCUC | GCAACUAACUAACUUAAUUA | 63.9 | 2.7495454 | 31.933333 | 1.8187297 |
| SEQ ID NO: 455) | | | | | | | |
| (SEQ ID NO: 567: | 501 | UAUUUUGAAUUAAGUUAGUUAG | AACUAACUUAAUUCAAAAUA | 98.266667 | 0.9171211 | 75.5 | 2.4826062 |
| SEQ ID NO: 456) | | | | | | | |
| (SEQ ID NO: 568: | 502 | UGAUUUUGAAUUAAGUUAGUUA | ACUAACUUAAUUCAAAAUCA | 87.2 | 2.4269322 | 48.4 | 3.4655447 |
| SEQ ID NO: 457) | | | | | | | |
| (SEQ ID NO: 569: | 505 | UGUUGAUUUUGAAUUAAGUUAG | AACUUAAUUCAAAAUCAACA | 102.56667 | 5.7623298 | 71.733333 | 2.9979623 |
| SEQ ID NO: 458) | | | | | | | |
| (SEQ ID NO: 7: | 544 | UUUUUAAGUGAAGUUACUUCUG | GAAGUAACUUCACUUAAAAA | 58.9 | 2.6388129 | 24.466667 | 1.8123036 |
| SEQ ID NO: 68) | | | | | | | |
| (SEQ ID NO: 36: | 572 | UAUGCUAUUAUCUUGUUUUUCU | AAAAACAAGAUAAUAGCAUA | 48.633333 | 3.2712553 | 17.4 | 2.1221059 |
| SEQ ID NO: 97) | | | | | | | |
| (SEQ ID NO: 570: | 633 | UUUGACUAUGCUGUUGGUUUAA | AAACCAACAGCAUAGUCAAA | 103.5 | 3.1432467 | 68.833333 | 1.3420548 |
| SEQ ID NO: 459) | | | | | | | |
| (SEQ ID NO: 571: | 634 | UUUUGACUAUGCUGUUGGUUUA | AACCAACAGCAUAGUCAAAA | 97.166667 | 8.5341145 | 83.366667 | 8.2561761 |
| SEQ ID NO: 460) | | | | | | | |
| (SEQ ID NO: 572: | 636 | UUAUUUGACUAUGCUGUUGGUU | CCAACAGCAUAGUCAAAUAA | 91.3 | 10.1 | 58.6 | 4.0648903 |
| SEQ ID NO: 461) | | | | | | | |
| (SEQ ID NO: 573: | 639 | UUUUUAUUUGACUAUGCUGUUG | ACAGCAUAGUCAAAUAAAAA | 101.53333 | 9.5729364 | 47 | 2.2854613 |
| SEQ ID NO: 462) | | | | | | | |
| (SEQ ID NO: 574: | 642 | UUUCUUUUAUUUGACUAUGCUG | GCAUAGUCAAAUAAAAGAAA | 124.33333 | 20.129277 | 45.466667 | 2.8759539 |
| SEQ ID NO: 463) | | | | | | | |
| (SEQ ID NO: 575: | 645 | UUAUUUCUUUUAUUUGACUAUG | UAGUCAAAUAAAAGAAAUAA | 107.9 | 8.2099533 | 49.3 | 4.2770706 |
| SEQ ID NO: 464) | | | | | | | |
| (SEQ ID NO: 576: | 646 | UCUAUUUCUUUUAUUUGACUAU | AGUCAAAUAAAAGAAAUAGA | 112.63333 | 5.1872707 | 56.2 | 10.759337 |
| SEQ ID NO: 465) | | | | | | | |
| (SEQ ID NO: 577: | 649 | UUUUCUAUUUCUUUUAUUUGAC | CAAAUAAAAGAAAUAGAAAA | 73.1 | 7.7323994 | 38.733333 | 2.9384425 |
| SEQ ID NO: 466) | | | | | | | |
| (SEQ ID NO: 578: | 651 | UAUUUUCUAUUUCUUUUAUUUG | AAUAAAAGAAAUAGAAAAUA | 75.3 | 2.0952327 | 43.2 | 1.4977761 |
| SEQ ID NO: 467) | | | | | | | |
| (SEQ ID NO: 579: | 654 | UCUGAUUUUCUAUUUCUUUUAU | AAAAGAAAUAGAAAAUCAGA | 89 | 6.4779112 | 56.166667 | 4.9670022 |
| SEQ ID NO: 468) | | | | | | | |
| (SEQ ID NO: 580: | 691 | UAAAUUUCUGUGGGUUCUUGAA | CAAGAACCCACAGAAAUUUA | 97.466667 | 9.619136 | 57.9 | 6.6161419 |
| SEQ ID NO: 469) | | | | | | | |
| (SEQ ID NO: 38: | 694 | UGAGAAAUUUCUGUGGGUUCUU | GAACCCACAGAAAUUUCUCA | 60.533333 | 4.4212869 | 26.566667 | 5.5798248 |
| SEQ ID NO; 99) | | | | | | | |
| (SEQ ID NO: 8: | 701 | UGAAGAUAGAGAAAUUUCUGUG | CAGAAAUUUCUCUAUCUUCA | 60.966667 | 2.2063041 | 23.066667 | 1.4110674 |
| SEQ ID NO: 69) | | | | | | | |
| (SEQ ID NO: 581: | 702 | UGGAAGAUAGAGAAAUUUCUGU | AGAAAUUUCUCUAUCUUCCA | 75.333333 | 1.8223916 | 42.3 | 2.0404248 |
| SEQ ID NO: 470) | | | | | | | |
| (SEQ ID NO: 582: | 762 | UUUUUACAUUUCUUAUUUCAUU | UGAAAUAAGAAAUGUAAAAA | 71.6 | 1.0440307 | 42.466667 | 7.7203915 |
| SEQ ID NO: 471) | | | | | | | |
| (SEQ ID NO: 583: | 763 | UGUUUUACAUUUCUUAUUUCAU | GAAAUAAGAAAUGUAAAACA | 60.266667 | 8.2049443 | 28.2 | 1.6802778 |
| SEQ ID NO: 472) | | | | | | | |
| (SEQ ID NO: 584: | 765 | UAUGUUUUACAUUUCUUAUUUC | AAUAAGAAAUGUAAAACAUA | 113.93333 | 6.6217655 | 71.2 | 6.512552 |
| SEQ ID NO: 473) | | | | | | | |
| (SEQ ID NO: 585: | 768 | UAUCAUGUUUUACAUUUCUUAU | AAGAAAUGUAAAACAUGAUA | 114.4 | 15.162124 | 84.766667 | 4.7178149 |
| SEQ ID NO: 474) | | | | | | | |
| (SEQ ID NO: 586: | 862 | UCAUGAAAAACUUGAGAGUUGC | AACUCUCAAGUUUUUCAUGA | 94.3 | 16.67643 | 44.8 | 4.9923274 |
| SEQ ID NO: 475) | | | | | | | |
| (SEQ ID NO: 587: | 932 | UAAGUUUUGUGAUCCAUCUAUU | UAGAUGGAUCACAAAACUUA | 61.733333 | 0.6741249 | 31.766667 | 0.6119187 |
| SEQ ID NO: 476) | | | | | | | |
| (SEQ ID NO: 588: | 939 | UUUCAUUGAAGUUUUGUGAUCC | AUCACAAAACUUCAAUGAAA | 78.2 | 4.1932485 | 42.7 | 2.5534291 |
| SEQ ID NO: 477) | | | | | | | |
| (SEQ ID NO: 589: | 941 | UGUUUCAUUGAAGUUUUGUGAU | CACAAAACUUCAAUGAAACA | 89.466667 | 3.2297231 | 67.466667 | 1.0898522 |
| SEQ ID NO: 478) | | | | | | | |
| (SEQ ID NO: 590: | 942 | UCGUUUCAUUGAAGUUUUGUGA | ACAAAACUUCAAUGAAACGA | 85.566667 | 4.6048284 | 83.266667 | 1.6374099 |
| SEQ ID NO: 479) | | | | | | | |
| (SEQ ID NO: 591: | 957 | UUUUGUAGUUCUCCCACGUUUC | AACGUGGGAGAACUACAAAA | 70.866667 | 2.5201411 | 56.233333 | 2.1073944 |
| SEQ ID NO: 480) | | | | | | | |
| (SEQ ID NO: 592: | 958 | UAUUUGUAGUUCUCCCACGUUU | ACGUGGGAGAACUACAAAUA | 69.2 | 0.6350853 | 53.933333 | 1.7071744 |
| SEQ ID NO: 481) | | | | | | | |
| (SEQ ID NO: 593: | 959 | UUAUUUGUAGUUCUCCCACGUU | CGUGGGAGAACUACAAAUAA | 71.3 | 1.0535654 | 51.033333 | 0.7310571 |
| SEQ ID NO: 482) | | | | | | | |
| (SEQ ID NO: 594: | 1011 | UGUAUAUCUUCUCUAGGCCCAA | GGGCCUAGAGAAGAUAUACA | 80.066667 | 3.3152845 | 56.766667 | 1.0898522 |
| SEQ ID NO: 483) | | | | | | | |
| (SEQ ID NO: 595: | 1012 | UAGUAUAUCUUCUCUAGGCCCA | GGCCUAGAGAAGAUAUACUA | 70.333333 | 14.174194 | 52.633333 | 5.1450084 |
| SEQ ID NO: 484) | | | | | | | |
| (SEQ ID NO: 43: | 1034 | UUAAUUAGAUUGCUUCACUAUG | UAGUGAAGCAAUCUAAUUAA | 52.6 | 1.1676187 | 21.833333 | 4.547649 |
| SEQ ID NO: 304) | | | | | | | |
| (SEQ ID NO: 596: | 1072 | UUGUUGUCUUUCCAGUCUUCCA | GAAGACUGGAAAGACAACAA | 87.7 | 1.1135529 | 54.8 | 1.7521415 |
| SEQ ID NO: 485) | | | | | | | |
| (SEQ ID NO: 597: | 1075 | UGUUUGUUGUCUUUCCAGUCUU | GACUGGAAAGACAACAAACA | 101.76667 | 2.8497563 | 81.633333 | 3.0333333 |
| SEQ ID NO: 486) | | | | | | | |
| (SEQ ID NO: 598: | 1077 | UAUGUUUGUUGUCUUUCCAGUC | CUGGAAAGACAACAAACAUA | 87.033333 | 1.8113838 | 56.7 | 1.8027756 |
| SEQ ID NO: 487) | | | | | | | |
| (SEQ ID NO: 10: | 1079 | UUAAUGUUUGUUGUCUUUCCAG | GGAAAGACAACAAACAUUAA | 55.666667 | 1.4449145 | 25.4 | 0.8082904 |
| SEQ ID NO: 71) | | | | | | | |
| (SEQ ID NO: 45: | 1082 | UAUAUAAUGUUUGUUGUCUUUC | AAGACAACAAACAUUAUAUA | 45.233333 | 2.2556103 | 17.866667 | 1.0397649 |
| SEQ ID NO: 106) | | | | | | | |
| (SEQ ID NO: 599: | 1083 | UAAUAUAAUGUUUGUUGUCUUU | AGACAACAAACAUUAUAUUA | 84.566667 | 5.500404 | 50.2 | 2.8448784 |
| SEQ ID NO: 488) | | | | | | | |
| (SEQ ID NO: 600: | 1086 | UUUCAAUAAAUGUUUGUUGUC | CAACAAACAUUAUAUUGAAA | 69.1266667 | 4.9599507 | 31.766667 | 0.6064468 |
| SEQ ID NO: 489) | | | | | | | |
| (SEQ ID NO: 601: | 1088 | UUAUUCAAUAUAAUGUUUGUUG | ACAAACAUUAUAUUGAAUAA | 58.4 | 3.1749016 | 31.366667 | 0.8819171 |
| SEQ ID NO: 490) | | | | | | | |
| (SEQ ID NO: 602: | 1089 | UAUAUUCAAUAUAAUGUUUGUU | CAAACAUUAUAUUGAAUAUA | 84.5 | 4.2524503 | 56.7 | 2.1221059 |
| SEQ ID NO: 491) | | | | | | | |
| (SEQ ID NO: 603: | 1093 | UAAGAAUAUUCAAUAUAAUGUU | CAUUAUAUUGAAUAUUCUUA | 84.866667 | 1.3245544 | 70.366667 | 1.3980145 |
| SEQ ID NO: 492) | | | | | | | |
| (SEQ ID NO: 46: | 1094 | UAAAGAAUAUUCAAUAUAAUGU | AUUAUAUUGAAUAUUCUUUA | 49.7 | 1.569501 | 25.3 | 1.3316656 |
| SEQ ID NO: 107) | | | | | | | |
| (SEQ ID NO: 604: | 1097 | UUAAAAAGAAUAUUCAAUAUAA | AUAUUGAAUAUUCUUUUUAA | 64.666667 | 0.7535103 | 43.7 | 12.219247 |
| SEQ ID NO: 493) | | | | | | | |
| (SEQ ID NO: 605: | 1098 | UGUAAAAAGAAUAUUCAAUAUA | UAUUGAAUAUUCUUUUUACA | 60.033333 | 3.0179095 | 35.566667 | 1.0088497 |
| SEQ ID NO: 494) | | | | | | | |
| (SEQ ID NO: 606: | 1120 | UAGUUGGUUUCGUGAUUUCCCA | GGAAAUCACGAAACCAACUA | 67.666667 | 2.4009257 | 61.2 | 1.7925773 |
| SEQ ID NO: 495) | | | | | | | |
| (SEQ ID NO: 607: | 1179 | UUUUGUUUUCCGGGAUUGCAUU | UGCAAUCCCGGAAAACAAAA | 95.633333 | 5.8356186 | 75.133333 | 6.4957251 |
| SEQ ID NO: 496) | | | | | | | |
| (SEQ ID NO: 608: | 1180 | UCUUUGUUUUCCGGGAUUGCAU | GCAAUCCCGGAAAACAAAGA | 112.66667 | 4.7301633 | 89.033333 | 7.1884939 |
| SEQ ID NO: 497) | | | | | | | |
| (SEQ ID NO: 609: | 1186 | UCCAAAUCUUUGUUUUCCGGGA | CCGGAAAACAAAGAUUUGGA | 108.9 | 3.5360053 | 68.2 | 3.5341194 |
| SEQ ID NO: 498) | | | | | | | |
| (SEQ ID NO: 610: | 1196 | UGUAGAAAACACCAAAUCUUUG | AAGAUUUGGUGUUUUCUACA | 83.866667 | 1.8675593 | 53.8 | 2.0502032 |
| SEQ ID NO: 499) | | | | | | | |
| (SEQ ID NO: 611: | 1199 | UCAAGUAGAAAACACCAAAUCU | AUUUGGUGUUUUCUACUUGA | 99.566667 | 1.3617799 | 50.4 | 3.9208843 |
| SEQ ID NO: 500) | | | | | | | |
| (SEQ ID NO: 612: | 1210 | UCUUUGUGAUCCCAAGUAGAAA | UCUACUUGGGAUCACAAAGA | 96.066667 | 2.8368605 | 65.5 | 0.9291573 |
| SEQ ID NO: 501) | | | | | | | |
| (SEQ ID NO: 613: | 1216 | UCUUUUGCUUUGUGAUCCCAAG | UGGGAUCACAAAGCAAAAGA | 96.733333 | 4.2451279 | 74.533333 | 5.7649323 |
| SEQ ID NO: 502) | | | | | | | |
| (SEQ ID NO: 614: | 1282 | UUGUUUUCUCCACACUCAUCAU | GAUGAGUGUGGAGAAAACAA | 100.1 | 8.2002032 | 69.433333 | 2.8695722 |
| SEQ ID NO: 503) | | | | | | | |
| (SEQ ID NO: 615: | 1283 | UUUGUUUUCUCCACACUCAUCA | AUGAGUGUGGAGAAAACAAA | 96.766667 | 5.2122718 | 61.5 | 3.3045423 |
| SEQ ID NO: 504) | | | | | | | |
| (SEQ ID NO: 616: | 1284 | UGUUGUUUUCUCCACACUCAUC | UGAGUGUGGAGAAAACAACA | 110.1 | 4.0016663 | 68.833333 | 4.8670091 |
| SEQ ID NO: 505) | | | | | | | |
| (SEQ ID NO: 617: | 1291 | UCAUUUAGGUUGUUUUCUCCAC | GGAGAAAACAACCUAAAUGA | 96.566667 | 6.0000926 | 55.833333 | 8.5958 |
| SEQ ID NO: 506) | | | | | | | |
| (SEQ ID NO: 618: | 1295 | UUUACCAUUUAGGUUGUUUUCU | AAAACAACCUAAAUGGUAAA | 118.76667 | 3.4594476 | 95.5 | 17.707155 |
| SEQ ID NO: 507) | | | | | | | |
| (SEQ ID NO: 619: | 1296 | UUUUACCAUUUAGGUUGUUUUC | AAACAACCUAAAUGGUAAAA | 109.8 | 2.7495454 | 80.233333 | 5.0273695 |
| SEQ ID NO: 508) | | | | | | | |
| (SEQ ID NO: 620: | 1297 | UAUUUACCAUUUAGGUUGUUUU | AACAACCUAAAUGGUAAAUA | 103.26667 | 3.2620716 | 68.366667 | 9.5202124 |
| SEQ ID NO: 509) | | | | | | | |
| (SEQ ID NO: 621: | 1299 | UAUAUUUACCAUUUAGGUUGUU | CAACCUAAAUGGUAAAUAUA | 91.366667 | 3.2199034 | 47.566667 | 5.1239091 |
| SEQ ID NO: 510) | | | | | | | |
| (SEQ ID NO: 622: | 1303 | UUGUUAUAUUUACCAUUUAGGU | CUAAAUGGUAAAUAUAACAA | 108.16667 | 13.339832 | 76.766667 | 5.7391443 |
| SEQ ID NO: 511) | | | | | | | |
| (SEQ ID NO: 623: | 1305 | UUUUGUUAUAUUUACCAUUUAG | AAAUGGUAAAUAUAACAAAA | 75.466667 | 5.3868152 | 41.533333 | 6.1723397 |
| SEQ ID NO: 512) | | | | | | | |
| (SEQ ID NO: 624: | 1306 | UGUUUGUUAUAUUUACCAUUUA | AAUGGUAAAUAUAACAAACA | 99.033333 | 9.7886556 | 81.766667 | 2.7412487 |
| SEQ ID NO: 513) | | | | | | | |
| (SEQ ID NO: 625: | 1376 | UGAGUAUAACCUUCCAUUUUGA | AAAAUGGAAGGUUAUACUCA | 75.1 | 2.0305993 | 56.466667 | 4.4175156 |
| SEQ ID NO: 514) | | | | | | | |
| (SEQ ID NO: 626: | 1380 | UUAUAGAGUAUAACCUUCCAUU | UGGAAGGUUAUACUCUAUAA | 75.933333 | 17.922456 | 31.133333 | 3.5025388 |
| SEQ ID NO: 515) | | | | | | | |
| (SEQ ID NO: 627: | 1384 | UAUUUUAUAGAGUAUAACCUUC | AGGUUAUACUCUAUAAAAUA | 64.533333 | 2.4767811 | 46.9 | 2.570992 |
| SEQ ID NO: 516) | | | | | | | |
| (SEQ ID NO: 628: | 1388 | UGUUGAUUUUAUAGAGUAUAAC | UAUACUCUAUAAAAUCAACA | 102 | 13.360514 | 98.333333 | 9.7813314 |
| SEQ ID NO: 517) | | | | | | | |
| (SEQ ID NO: 629: | 1394 | UAUUUUGGUUGAUUUUAUAGAG | CUAUAAAAUCAACCAAAAUA | 69.833333 | 6.5973058 | 58.766667 | 1.449521 |
| SEQ ID NO: 518) | | | | | | | |
| (SEQ ID NO: 630: | 1426 | UCAAAGCUUUCUGAAUCUGUUG | ACAGAUUCAGAAAGCUUUGA | 62.366667 | 1.6333333 | 46.1 | 1.5 |
| SEQ ID NO: 519) | | | | | | | |
| (SEQ ID NO: 631: | 1434 | UAGUUCAUUCAAAGCUUUCUGA | AGAAAGCUUUGAAUGAACUA | 92.833333 | 3.9295179 | 54.666667 | 3.9252742 |
| SEQ ID NO: 520) | | | | | | | |
| (SEQ ID NO: 632: | 1447 | UUUAAAUUUGCCUCAGUUCAUU | UGAACUGAGGCAAAUUUAAA | 66.866667 | 2.5260861 | 29.566667 | 1.3119112 |
| SEQ ID NO: 521) | | | | | | | |
| (SEQ ID NO: 633: | 1448 | UUUUAAAUUUGCCUCAGUUCAU | GAACUGAGGCAAAUUUAAAA | 66.933333 | 3.5295577 | 30.566667 | 1.2333333 |
| SEQ ID NO: 522) | | | | | | | |

TABLE 3
Exemplary Sequences and Their Calculated Absolute
IC50 Values by Dose Response in Primary Hepatocytes

| | Free uptake in primary | | Free uptake in primary | |
|---|---|---|---|---|
| | cynomolgus hepatocytes (PCH) | | human hepatocytes (PHH) | |
| | Log | Absolute | Log | Absolute |
| SEQ ID NOS: | (concentration) | IC50 (nM) | (concentration) | IC50 (nM) |
| (SEQ ID NO: 278: SEQ ID NO: 134) | −0.284 | 0.52 | 0.160 | 1.45 |
| (SEQ ID NO: 19: SEQ ID NO: 80) | 0.135 | 1.36 | 0.469 | 2.94 |
| (SEQ ID NO: 24: SEQ ID NO: 85) | 0.057 | 1.14 | 0.652 | 4.49 |
| (SEQ ID NO: 30: SEQ ID NO: 91) | 0.111 | 1.29 | 0.574 | 3.75 |
| (SEQ ID NO: 31: SEQ ID NO: 92) | −0.064 | 0.86 | 0.282 | 1.91 |
| (SEQ ID NO: 62: SEQ ID NO: 123) | 0.201 | 1.59 | 0.253 | 1.79 |
| (SEQ ID NO: 9: SEQ ID NO: 70) | −0.284 | 0.52 | −0.152 | 0.71 |
| (SEQ ID NO: 15: SEQ ID NO: 76) | −0.355 | 0.44 | −0.429 | 0.37 |
| (SEQ ID NO: 2: SEQ ID NO: 63) | 0.411 | 2.58 | 0.050 | 1.12 |
| (SEQ ID NO: 17: SEQ ID NO: 78) | 0.039 | 1.09 | 0.204 | 1.60 |
| (SEQ ID NO: 20: SEQ ID NO: 81) | 0.126 | 1.34 | 0.529 | 3.38 |
| (SEQ ID NO: 21: SEQ ID NO: 82) | 0.119 | 1.32 | 0.432 | 2.71 |
| (SEQ ID NO: 22: SEQ ID NO: 83) | 0.050 | 1.12 | 0.300 | 2.00 |
| (SEQ ID NO: 23: SEQ ID NO: 84) | 0.539 | 3.46 | 0.218 | 1.65 |
| (SEQ ID NO: 530: SEQ ID NO: 419) | 0.741 | 5.51 | 0.758 | 5.73 |
| (SEQ ID NO: 25: SEQ ID NO: 86) | 0.137 | 1.37 | 0.436 | 2.73 |
| (SEQ ID NO: 4: SEQ ID NO: 65) | 0.500 | 3.16 | 0.550 | 3.55 |
| (SEQ ID NO: 26: SEQ ID NO: 87) | 0.485 | 3.05 | 0.427 | 2.67 |
| (SEQ ID NO: 28: SEQ ID NO: 89) | −0.381 | 0.42 | 0.202 | 1.59 |
| (SEQ ID NO: 29: SEQ ID NO: 90) | 0.137 | 1.37 | 0.509 | 3.23 |
| (SEQ ID NO: 5: SEQ ID NO: 66) | 0.214 | 1.64 | 0.440 | 2.75 |
| (SEQ ID NO: 558: SEQ ID NO: 447) | 0.413 | 2.59 | 0.396 | 2.49 |
| (SEQ ID NO: 60: SEQ ID NO: 121) | 0.491 | 3.10 | 0.468 | 2.94 |
| (SEQ ID NO: 61: SEQ ID NO: 122) | 0.181 | 1.52 | 0.369 | 2.34 |
| (SEQ ID NO: 34: SEQ ID NO: 95) | −0.093 | 0.81 | −0.077 | 0.84 |
| (SEQ ID NO: 7: SEQ ID NO: 68) | 0.288 | 1.94 | −0.230 | 0.59 |
| (SEQ ID NO: 36: SEQ ID NO: 97) | −0.015 | 0.97 | −0.187 | 0.65 |
| (SEQ ID NO: 43: SEQ ID NO: 104) | −0.346 | 0.45 | −0.238 | 0.58 |
| (SEQ ID NO: 10: SEQ ID NO: 71) | −0.252 | 0.56 | −0.017 | 0.96 |
| (SEQ ID NO: 45: SEQ ID NO: 106) | −0.268 | 0.54 | −0.116 | 0.77 |
| (SEQ ID NO: 46: SEQ ID NO: 107) | −0.003 | 0.99 | 0.519 | 3.31 |
| (SEQ ID NO: 18: SEQ ID NO: 79) | −0.151 | 0.71 | 0.181 | 1.52 |
| (SEQ ID NO: 296: SEQ ID NO: 152) | 0.418 | 2.62 | 0.821 | 6.62 |
| (SEQ ID NO: 11: SEQ ID NO: 72) | 0.055 | 1.13 | 0.091 | 1.23 |
| (SEQ ID NO: 278: SEQ ID NO: 134) | −0.284 | 0.52 | 0.160 | 1.45 |

TABLE 4
Exemplary Sequences of the Present Application
and Their in vivo Potency in Silencing Human Angptl3 mRNA

| Guide | | Passenger | | % of gene | |
|---|---|---|---|---|---|
| Strand | | Strand | | remaining at | |
| SEQ | Guide strand | SEQ | Passenger strand | 0.5 mg/kg | |
| ID NOs | sequence (5′-3′) | ID NOs | sequence (5′-3′) | Mean | SD |
| 18 | UAAAUCUUGAUUUUGGCUCUGG | 79 | AGAGCCAAAAUCAAGAUUUA | 74.39 | 9.502 |
| 278 | UUAGCAAAUCUUGAUUUUGGCU | 134 | CCAAAAUCAAGAUUUGCUAA | 73.8 | 9.193 |
| 19 | UCAUAGCAAAUCUUGAUUUUGG | 80 | AAAAUCAAGAUUUGCUAUGA | 60.27 | 7.703 |
| 296 | UGACUGAUCAAAUAUGUUGAGU | 152 | UCAACAUAUUUGAUCAGUCA | 84.18 | 9.553 |
| 296 | UGACUGAUCAAAUAUGUUGAGU | 152 | UCAACAUAUUUGAUCAGUCA | 75.23 | 17.3 |
| 24 | UGAUAGAUCAUAAAAAGACUGA | 85 | AGUCUUUUUAUGAUCUAUCA | 58.71 | 11.97 |
| 30 | UUUAAGUUAGUUAGUUGCUCUU | 91 | GAGCAACUAACUAACUUAAA | 53.96 | 6.406 |
| 31 | UAUUAAGUUAGUUAGUUGCUCU | 92 | AACAGCAUAGUCAAAUAAAA | 51 | 17.42 |
| 62 | UUUUAUUUGACUAUGCUGUUGG | 123 | AACAGCAUAGUCAAAUAAAA | 52.65 | 13.27 |
| 62 | UUUUAUUUGACUAUGCUGUUGG | 123 | AACAGCAUAGUCAAAUAAAA | 68.9 | 10.23 |
| 9 | UGAAAAACUUGAGAGUUGCUGG | 70 | AGCAACUCUCAAGUUUUUCA | 82.52 | 12.94 |
| 11 | UGUAUAACCUUCCAUUUUGAGA | 72 | UCAAAAUGGAAGGUUAUACA | 76.28 | 4.298 |
| 15 | UAUAAAAAGAAGGAGCUUAAUU | 76 | UUAAGCUCCUUCUUUUUAUA | 79.86 | 12.09 |
| 2 | UCAAUAAAAAGAAGGAGCUUAA | 63 | AAGCUCCUUCUUUUUAUUGA | 63.26 | 9.028 |
| 17 | UAAAUAACUAGAGGAACAAUAA | 78 | AUUGUUCCUCUAGUUAUUUA | 65.58 | 10.09 |
| 20 | UUUGAUUUUGGCUCUGGAGAUA | 81 | UCUCCAGAGCCAAAAUCAAA | 53.58 | 10.27 |
| 21 | UAUCGUCUAACAUAGCAAAUCU | 82 | AUUUGCUAUGUUAGACGAUA | 75.71 | 4.106 |
| 22 | UAUUUUUACAUCGUCUAACAUA | 83 | UGUUAGACGAUGUAAAAAUA | 70.01 | 10.57 |
| 23 | UAAAAUUUUUACAUCGUCUAAC | 84 | UAGACGAUGUAAAAAUUUUA | 51.07 | 4.74 |
| 530 | UUAAAAUUUUUACAUCGUCUAA | 419 | AGACGAUGUAAAAAUUUUAA | 60.36 | 13.02 |
| 25 | UUAAAAAGACUGAUCAAAUAUG | 86 | UAUUUGAUCAGUCUUUUUAA | 56.46 | 4.632 |
| 4 | UAUAAAAAGACUGAUCAAAUAU | 65 | AUUUGAUCAGUCUUUUUAUA | 52.22 | 5.367 |
| 26 | UAUAGAUCAUAAAAAGACUGAU | 87 | CAGUCUUUUUAUGAUCUAUA | 66.88 | 13.51 |
| 28 | UUUUAUAUGUAGUUCUUCUCAG | 89 | GAGAAGAACUACAUAUAAAA | 60.78 | 17.33 |
| 29 | UAUUUUUGACUUGUAGUUUAUA | 90 | UAAACUACAAGUCAAAAAUA | 61.32 | 17.12 |
| 5 | UGACAUAUUCUUUACCUCUUCA | 66 | AAGAGGUAAAGAAUAUGUCA | 48.7 | 5.69 |
| 558 | UAGAAUUUUUUCUUCUAGGAGG | 447 | UCCUAGAAGAAAAAAUUCUA | 49.84 | 10.66 |
| 60 | UUUGUUGAAGUAGAAUUUUUUC | 121 | AAAAAUUCUACUUCAACAAA | 43.72 | 4.574 |
| 61 | UUUCACUUUUUGUUGAAGUAGA | 122 | UACUUCAACAAAAAGUGAAA | 47.28 | 10.15 |
| 34 | UUUAGUUAGUUGCUCUUCUAAA | 95 | UAGAAGAGCAACUAACUAAA | 51.34 | 8.448 |
| 7 | UUUUUAAGUGAAGUUACUUCUG | 68 | GAAGUAACUUCACUUAAAAA | 54.32 | 14.11 |
| 36 | UAUGCUAUUAUCUUGUUUUUCU | 97 | AAAAACAAGAUAAUAGCAUA | 60.18 | 23.03 |
| 43 | UUAAUUAGAUUGCUUCACUAUG | 104 | UAGUGAAGCAAUCUAAUUAA | 53.12 | 7.828 |
| 10 | UUAAUGUUUGUUGUCUUUCCAG | 71 | GGAAAGACAACAAACAUUAA | 85.52 | 25.14 |
| 45 | UAUAUAAUGUUUGUUGUCUUUC | 106 | AAGACAACAAACAUUAUAUA | 43.89 | 6.124 |
| 46 | UAAAGAAUAUUCAAUAUAAUGU | 107 | AUUAUAUUGAAUAUUCUUUA | 48.36 | 6.407 |

TABLE 5
Exemplary Sequences of the Present Disclosures and
Their in vivo Dose Response in Silencing Human Angptl3 mRNA

| | | | | % mRNA | | % mRNA | | % mRNA | |
|---|---|---|---|---|---|---|---|---|---|
| Guide | Passenger | | | remaining | | remaining | | remaining | |
| Strand | Strand | | | at | | at | | at | |
| SEQ | SEQ | Guide strand | Passenger strand | 0.25 mg/kg | | 0.5 mg/kg | | 1 mg/kg | |
| ID NOs | ID NOs | sequence (5′-3′) | sequence (5′-3′) | Mean | SD | Mean | SD | Mean | SD |
| 18 | 79 | UAAAUCUUGAUUUUGGCUCUGG | AGAGCCAAAAUCAAGAUUUA | 89.2 | 14.4 | 84.8 | 13.6 | 62.6 | 10.6 |
| 278 | 134 | UUAGCAAAUCUUGAUUUUGGCU | CCAAAAUCAAGAUUUGCUAA | 81.24 | 11.83 | 72.4 | 9.5 | 53.9 | 7.6 |
| 19 | 80 | UCAUAGCAAAUCUUGAUUUUGG | AAAAUCAAGAUUUGCUAUGA | 71.63 | 8.30 | 32.1 | 7 3 | 25.6 | 9.0 |
| 48 | 109 | UUUUACAUCGUCUAACAUAGCA | CUAUGUUAGACGAUGUAAAA | 65.31 | 25.23 | 54.3 | 16.1 | 55.2 | 5.5 |
| 3 | 64 | UAAAUUUUUACAUCGUCUAACA | UUAGACGAUGUAAAAAUUUA | 81.92 | 14.35 | 61.7 | 6.1 | 57.1 | 8.2 |
| 296 | 152 | UGACUGAUCAAAUAUGUUGAGU | UCAACAUAUUUGAUCAGUCA | 94.31 | 25.87 | 76.6 | 13.1 | 60.9 | 12.5 |
| 24 | 85 | UGAUAGAUCAUAAAAAGACUGA | AGUCUUUUUAUGAUCUAUCA | 70.19 | 16.52 | 46.1 | 5.6 | 39.5 | 7.1 |
| 54 | 115 | UUUAUAUGUAGUUCUUCUCAGU | UGAGAAGAACUACAUAUAAA | 51.01 | 11.37 | 56.7 | 9.7 | 57.1 | 9.5 |
| 56 | 117 | UUUGAGUUGAGUUCAAGUGACA | UCACUUGAACUCAACUCAAA | 77.69 | 20.94 | 60.0 | 12.3 | 48.8 | 5.0 |
| 316 | 172 | UAAUUUUUUCUUCUAGGAGGCU | CCUCCUAGAAGAAAAAAUUA | 78.73 | 14.30 | 84.0 | 22.3 | 74.8 | 9.4 |
| 57 | 118 | UAAGUUAGUUAGUUGCUCUUCU | AAGAGCAACUAACUAACUUA | 84.54 | 17.77 | 62.6 | 17.5 | 46.7 | 14.6 |
| 30 | 91 | UUUAAGUUAGUUAGUUGCUCUU | GAGCAACUAACUAACUUAAA | 68.26 | 20.91 | 84.0 | 23.8 | 72.3 | 18.0 |
| 58 | 119 | UGAAUUAAGUUAGUUAGUUGCU | CAACUAACUAACUUAAUUCA | 89.70 | 10.35 | 38.6 | 7.9 | 46.6 | 9.7 |
| 6 | 67 | UUUUAAGUGAAGUUACUUCUGG | AGAAGUAACUUCACUUAAAA | 80.06 | 43.88 | 53.1 | 14.8 | 87.3 | 51.1 |
| 11 | 72 | UGUAUAACCUUCCAUUUUGAGA | UCAAAAUGGAAGGUUAUACA | 127.61 | 23.10 | 34.0 | 11.6 | 36.4 | 11.5 |
| 12 | 73 | UUUUAUAGAGUAUAACCUUCCA | GAAGGUUAUACUCUAUAAAA | 39.2 | 18.6 | 32.9 | 10.2 | 26.0 | 10.8 |

TABLE 6
Exemplary Sequences of the Present Application and
Their in vivo Potency and Duration Evaluations in Silencing Human Angplt3
mRNA and Reducing Serum Protein in Humanized Transgenic Mice

| | | | | % mRNA | | serum protein | |
|---|---|---|---|---|---|---|---|
| Guide | Passenger | | | remaining | | levels (ng/ml) | |
| Strand | Strand | | | on day 14 | | on day 14 | |
| SEQ | SEQ | Guide strand | Passenger strand | at 0.5 mg/kg | | at 0.5 mg/kg | |
| ID NOs | ID NOs | sequence (5′-3′) | sequence (5′-3′) | Mean | SEM | Mean | SEM |
| 278 | 134 | UUAGCAAAUCUUGAUUUUGGCU | CCAAAAUCAAGAUUUGCUAA | 27.50 | 2.65 | 17.51 | 2.47 |
| 19 | 80 | UCAUAGCAAAUCUUGAUUUUGG | AAAAUCAAGAUUUGCUAUGA | 30.50 | 9.57 | 14.35 | 2.26 |
| 48 | 109 | UUUUACAUCGUCUAACAUAGCA | CUAUGUUAGACGAUGUAAAA | 101.50 | 15.97 | 52.53 | 6.73 |
| 3 | 64 | UAAAUUUUUACAUCGUCUAACA | UUAGACGAUGUAAAAAUUUA | 139.50 | 20.34 | 68.65 | 4.66 |
| 24 | 85 | UGAUAGAUCAUAAAAAGACUGA | AGUCUUUUUAUGAUCUAUCA | 46.50 | 7.19 | 26.63 | 3.33 |
| 54 | 115 | UUUAUAUGUAGUUCUUCUCAGU | UGAGAAGAACUACAUAUAAA | 66.25 | 4.19 | 32.69 | 7.28 |
| 27 | 88 | UUAGUUUAUAUGUAGUUCUUCU | AAGAACUACAUAUAAACUAA | 32.25 | 9.39 | 17.35 | 2.85 |
| 55 | 116 | UCAAGUGACAUAUUCUUUACCU | GUAAAGAAUAUGUCACUUGA | 131.50 | 11.96 | 57.20 | 9.59 |
| 56 | 117 | UUUGAGUUGAGUUCAAGUGACA | UCACUUGAACUCAACUCAAA | 60.75 | 21.61 | 36.62 | 3.66 |
| 30 | 91 | UUUAAGUUAGUUAGUUGCUCUU | GAGCAACUAACUAACUUAAA | 113.30 | 18.91 | 49.54 | 6.51 |
| 31 | 92 | UAUUAAGUUAGUUAGUUGCUCU | AGCAACUAACUAACUUAAUA | 90.25 | 16.26 | 51.36 | 9.89 |
| 62 | 123 | UUUUAUUUGACUAUGCUGUUGG | AACAGCAUAGUCAAAUAAAA | 152.80 | 19.40 | 57.56 | 10.48 |
| 9 | 70 | UGAAAAACUUGAGAGUUGCUGG | AGCAACUCUCAAGUUUUUCA | 75.25 | 19.38 | 32.54 | 4.04 |
| 41 | 102 | UUUGAAUUAAUGUCCAUGGACU | UCCAUGGACAUUAAUUCAAA | 72.50 | 11.90 | 34.27 | 3.25 |
| 4 | 65 | UAUAAAAAGACUGAUCAAAUAU | AUUUGAUCAGUCUUUUUAUA | 61.50 | 12.23 | 38.09 | 4.65 |
| 5 | 66 | UGACAUAUUCUUUACCUCUUCA | AAGAGGUAAAGAAUAUGUCA | 69.75 | 7.23 | 49.87 | 11.49 |
| 558 | 447 | UAGAAUUUUUUCUUCUAGGAGG | UCCUAGAAGAAAAAAUUCUA | 85.25 | 14.84 | 50.98 | 8.71 |
| 60 | 121 | UUUGUUGAAGUAGAAUUUUUUC | AAAAAUUCUACUUCAACAAA | 89.75 | 18.82 | 54.25 | 11.35 |
| 61 | 122 | UUUCACUUUUUGUUGAAGUAGA | UACUUCAACAAAAAGUGAAA | 94.75 | 11.15 | 49.20 | 3.87 |
| 34 | 95 | UUUAGUUAGUUGCUCUUCUAAA | UAGAAGAGCAACUAACUAAA | 90.50 | 13.23 | 56.26 | 14.83 |
| 10 | 71 | UUAAUGUUUGUUGUCUUUCCAG | GGAAAGACAACAAACAUUAA | 46.00 | 8.08 | 34.03 | 6.33 |

### EXAMPLES

### Example 1: Design and Testing of siRNA Compounds Against ANGPTL-3 mRNA

The example described herein determined the potency of the siRNA compounds against human Angpt13 mRNA (Table 2, Set 1 and Set 2).

### Materials and Methods

### In Vitro Compound Screening

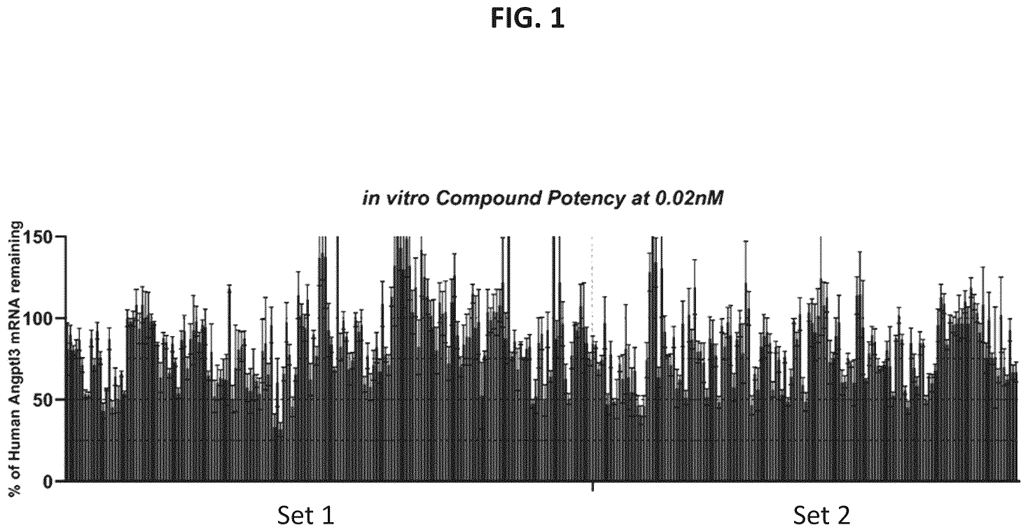

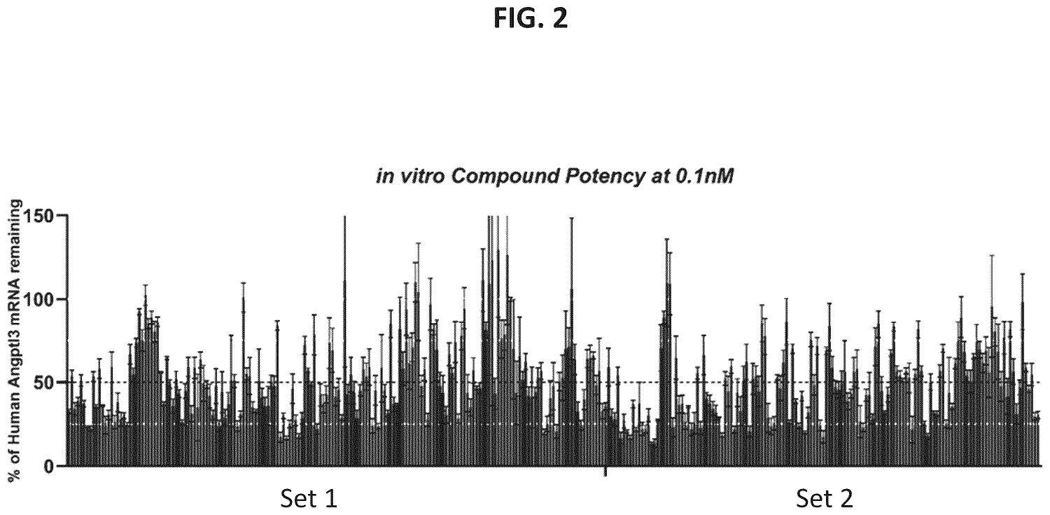

Two sets of siRNAs compounds against human Angpt13 transcript (Accession No: NM_014495.4) were designed. The first set included 176 compounds (Table 2, Set 1) and the second set included 141 compounds (Table 2, Set 2). The compounds were diluted into the desired concentration with PBS. The compounds were transfected into the cultured Huh-7 cells with Lipofectamine RNAiMAX (Invitrogen-13778-150) reagents. Each compound was tested at two concentrations of 0.02 nM (FIG. 1) and 0.1 nM (FIG. 2). At 24 hours post transfection, mRNA was extracted from the transfected cells using RNeasy 96 kit (Qiagen-74182). TaqMan RT-qPCR gene expression assay was conducted to analyze the compound potency in silencing Angplt3 mRNA.

### Ex Vivo Potency Evaluation in Primary Human Hepatocytes (PHH) and Primary Cynomolgus Monkey Hepatocytes (PCH)

Compounds with GalNAc conjugations were tested through free-uptake in primary human hepatocytes (PHH) or primary cynomolgus hepatocytes (PCH) (Table 3). The compounds were directly added to the cultured primary hepatocytes at 8 doses (0.0013 nM, 0.0064 nM, 0.0320 nM, 0.1600 nM, 0.8000 nM, 4.0000 nM, 20.0000 nM, 100.0000 nM). 48 hrs later, the cells were harvested for mRNA analysis through RT-qPCR. The absolute IC50 was calculated with three parameters log (inhibitor) vs. response equations, as shown in Table 3.

### In Vivo Compound Screening in Hydrodynamic Injection (HDI) Mouse Liver

Selected siRNA compounds with GalNAc conjugations were dosed on day 1 through subcutaneous dosing to BALB/c female animals (6˜8 weeks old). Animals then received 10 μg of pcDNA3.1-hsAngpt13 plasmids on day 4. Liver biopsies were taken on day5 for mRNA remaining analysis through RT-qPCR.

In Vivo Potency and Duration Evaluation in hsAngpt13 Transgenic Mice

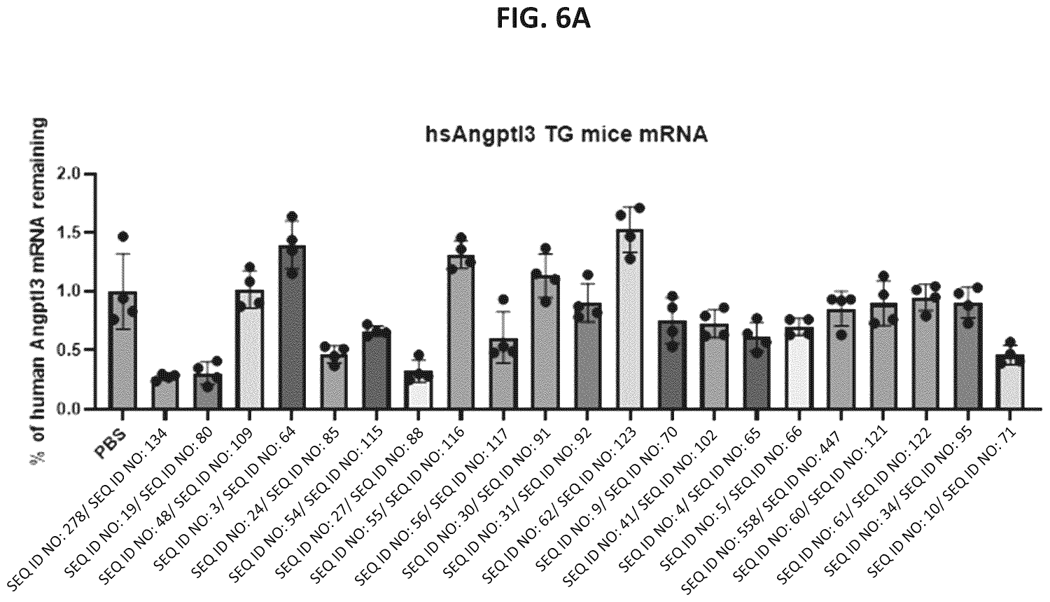

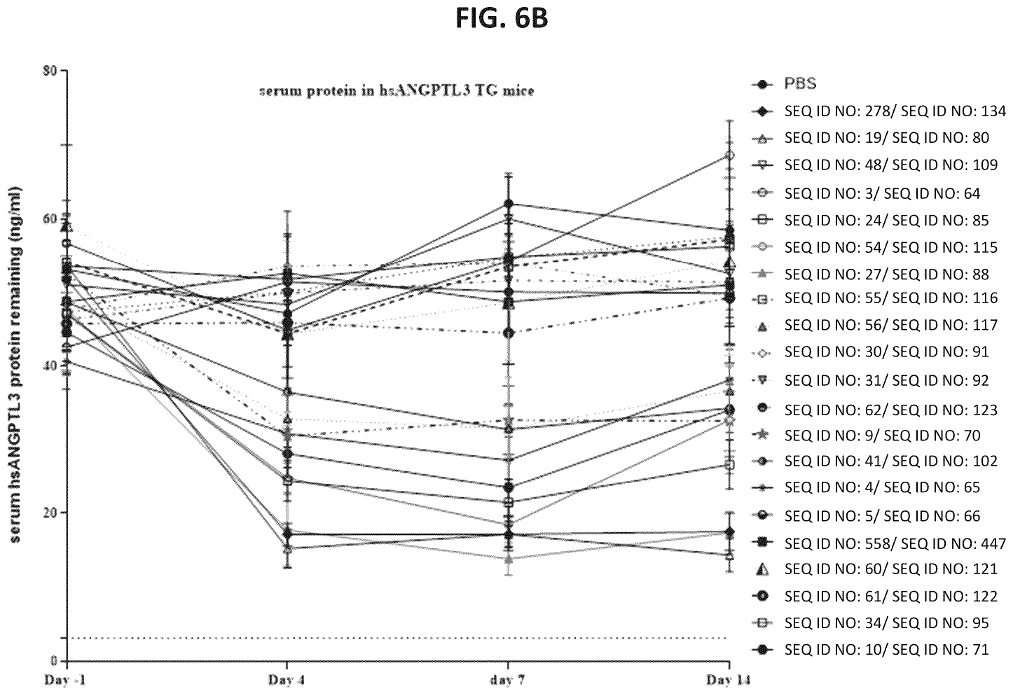

The hsAngpt13 transgenic mice is a humanized mice model that replaced the endogenous wildtype mouse Angpt13 alleles with human Angpt13 coding regions, introns, and 3′UTR. The mouse promoter regions and 5′UTR remain unchanged. Selected siRNA compounds with proprietary chemical modifications and GalNAc conjugates (FIGS. 6A-6B) were dosed into the hsAngpt13 TG mice through subcutaneous dosing at 1 mg/kg. The liver biopsies were taken on day 14 post dosing for mRNA analysis through RT-qPCR (FIG. 6A). Serum hsANGPTL3 protein levels were analyzed on day −1, 4, 7, and 14 through ELISA (R&D DANL30 ELISA kit) (FIG. 6B).

### Results and Observations

The percentage of human Angpt13 mRNA remaining in cultured Huh-7 cells relative to mock transfection when normalized to Gapdh mRNA levels, was determined for each compound following transfection into cells at a concentration of either 0.02 nM (FIG. 1) or 0.1 nM (FIG. 2). The results identified several compounds that were able to reduce the level of human Angpt13 mRNA in transfected cells by 20% to 50% or more than 50% or less at a concentration of 0.02 nM as (see Table 2, Set 1 and Set 2, and FIG. 1). Also, several compounds were able to reduce the level of human Angpt13 mRNA in transfected cells by between 50% to 75% or more than 75% or less at a concentration of 0.1 nM (see Table 2, Set 1 and Set 2, and FIG. 2).

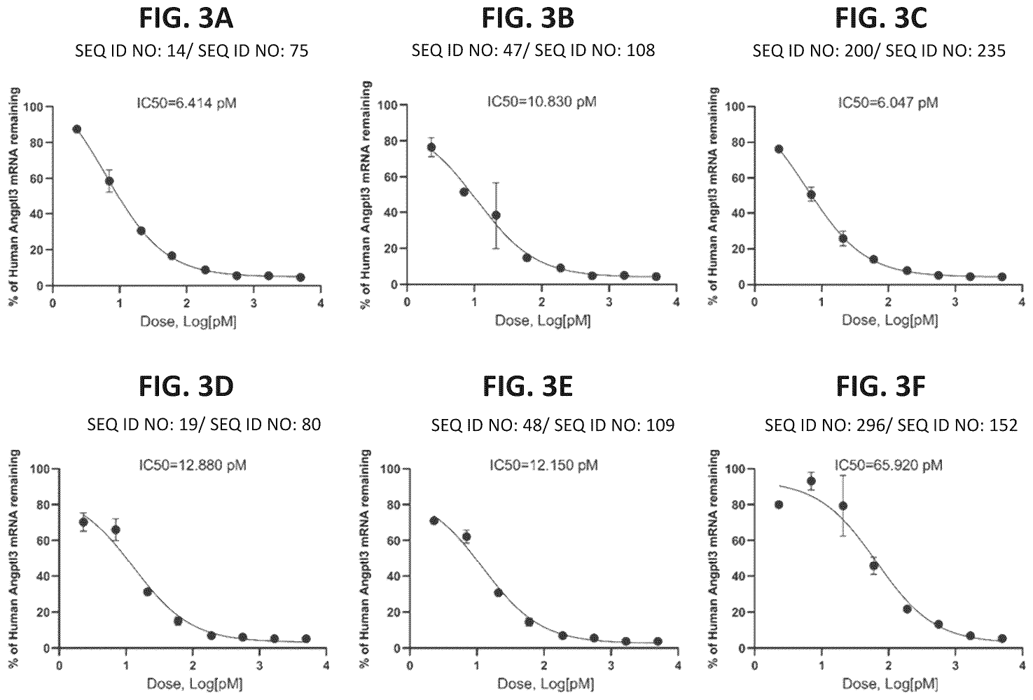

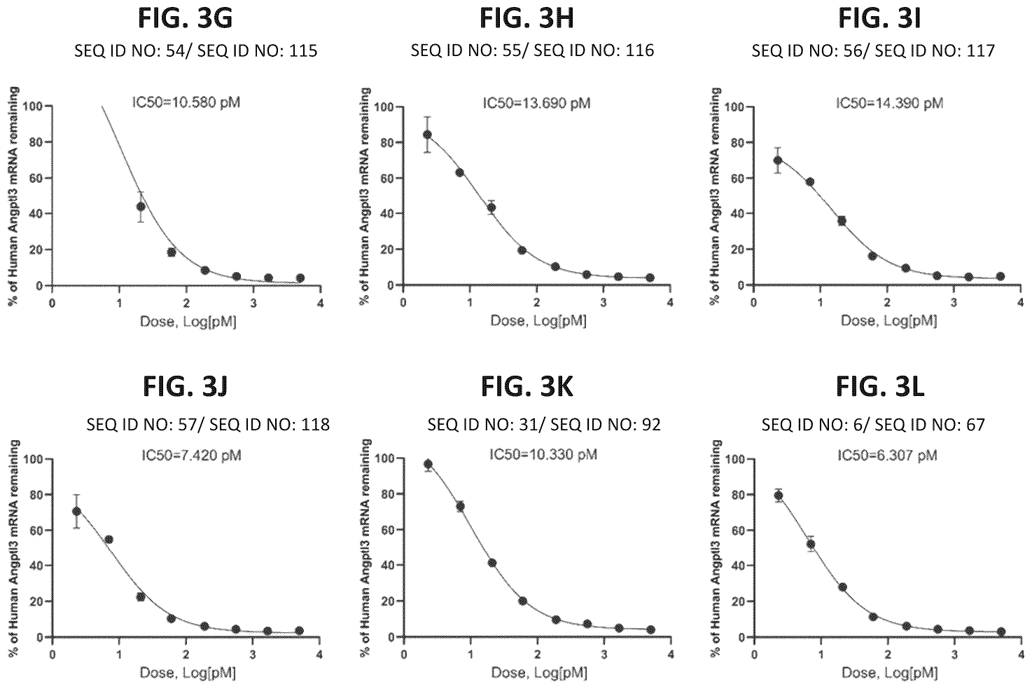

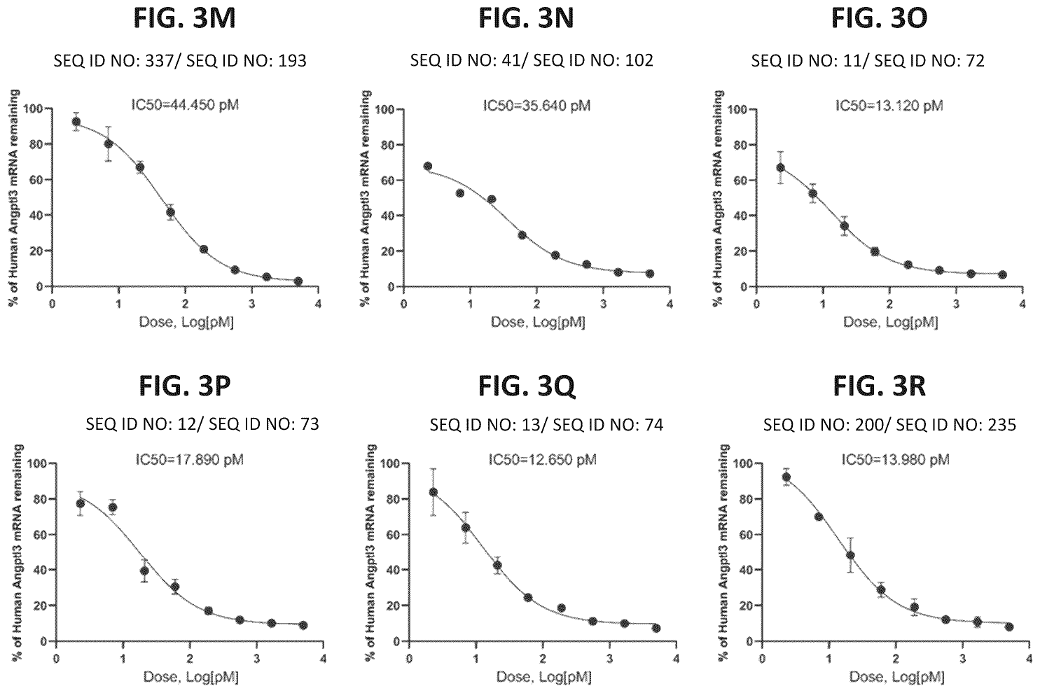

Based on the potency, sixty-two siRNA compounds were identified and selected (shown with bold in Table 2). The IC₅₀ values of eighteen of the selected compounds was determined, among which fifteen were shown to reduce the percentage of Angpt13 mRNA in cells by 50% at a dose of less than 20 pM (see FIGS. 3A-3R). For determining the IC50 value of the specific compounds, the Huh-7 cells were transfected with the indicated compounds at 2.3, 7, 21, 60, 190, 560, 1670, and 5000 picomolar concentration. IC50 was calculated using the three parameter Log (inhibitor) vs. response formula. As shown in FIGS. 3A-3R, data is presented as % of human Angpt13 mRNA remaining relative to Mock transfection when normalized to Gapdh mRNA levels (Mean, +/−SEM).

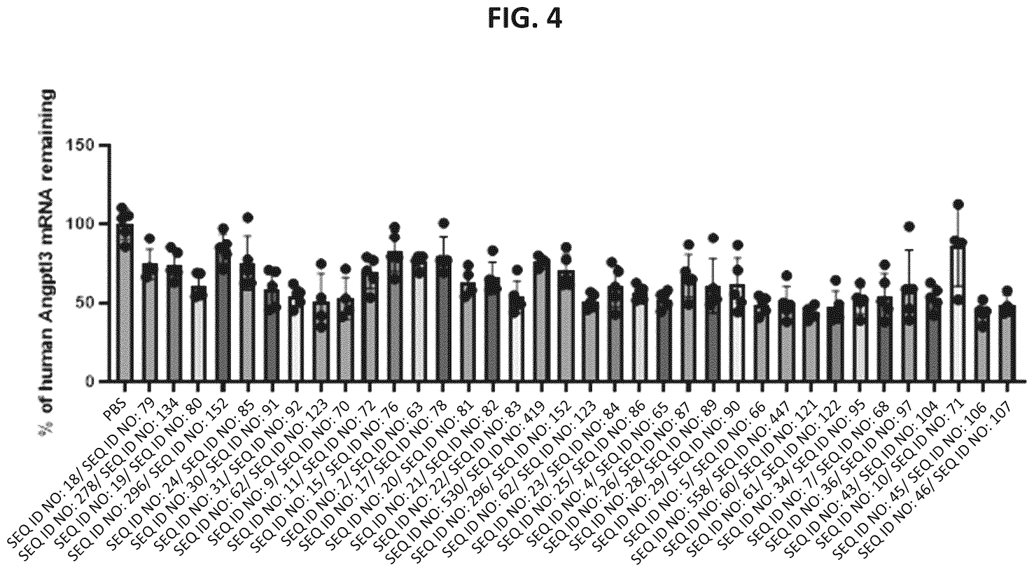

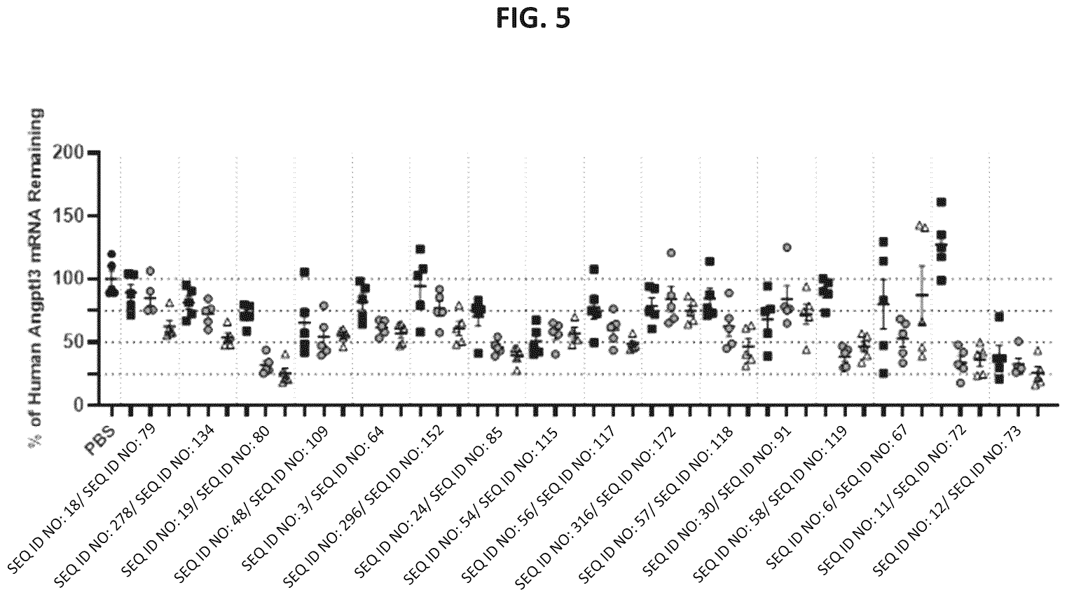

To measure in vivo potency of compounds listed in Table 2, Set 1 and Set 2, a hydrodynamic injection (HDI) mouse model was used for selected siRNA compounds containing GalNAc conjugations. Compounds were administered to BALB/c female mice (˜6-8 weeks old) at 0.5 mg/kg through subcutaneous dosing on day 1. On day 4, post-dosing, 10 μg of pcDNA3.1-hsAngpt13 plasmids were dosed through HDI and mRNA expression was examined (FIG. 4). Liver biopsies were taken on day 5 and the amount of mRNA remaining was analyzed via RT-qPCR. Data is represented as % of human Angpt13 mRNA remaining relative to PBS groups when normalized to Neomycin-resistant (NeoR) gene mRNA levels (Mean, +/−SD) (FIG. 4 and Table 4).

The in vivo dose response of selected siRNA compounds (Table 2, Set 1 and Set 2) was determined, as shown in FIG. 5. Compounds were dosed at 0.25 mg/kg (solid squares), 0.5 mg/kg (grey circles), or 1 mg/kg (triangles) subcutaneously on day 1. 10 μg of pcDNA3.1-hsAngpt13 plasmids were dosed through hydrodynamic injection (HDI) on day 4 post-dosing. Liver biopsies were taken on day 5 and the amount of mRNA remaining was analyzed via RT-qPCR. Data is represented as % of human Angpt13 mRNA remaining relative to PBS groups when normalized to NeoR mRNA levels (Mean, +/−SD) (FIG. 5 and Table 5).

The compound potency and duration evaluations of selected siRNA compounds (Table 2, Set 1 and Set 2) was evaluated using a humanized transgenic mice model that expresses human Angplt3, as shown in FIGS. 6A-6B and Table 6. Compounds were dosed at 1 mg/kg through subcutaneous dosing on day 0. Liver hsANGPTL3 mRNA levels were determined on day 14 (FIG. 6A and Table 6) and are represented as % of remaining relative to PBS group. Serum ANGPLT3 proteins concentrations were evaluated compared to PBS control (FIG. 6B and Table 6) and are represented as absolute values in ng/ml.

Several compounds were able to reduce the level of Angplt3 mRNA in transfected cells by between 20% to 50%, or greater than 50%, at either 0.02 nM or 0.1 nM, or both concentrations. Several compounds were able to reduce the level of Angplt3 mRNA in transfected cells by between 20% to 50%, by between 50% to 75%, or greater than 75%, at 0.1 nM.

Additionally, this work identifies several compounds that are able to reduce the level of Angplt3 mRNA in the liver in vivo. Specifically, multiple compounds are able to Angplt3 mRNA in vivo between 20% to 50%, or greater than 50%, when administered at 0.25 mg/kg, or 0.5 mg/kg, or 1 mg/kg, or a combination of these concentrations.

This work also identifies siRNA compounds that are able to reduce the level of human Angplt3 mRNA by between 9% and 72% in vivo and reduce the level of serum protein between 20% to 50% or greater than 50% in transgenic humanized mice

In summary, the present disclosure identifies numerous siRNA compounds that reduce the level of Angpt13 mRNA in target cells in vitro and in vivo and reduce serum protein levels in vivo.

1. An isolated oligonucleotide comprising a sense strand and an anti-sense strand, wherein:
the sense strand comprises a nucleotide sequence that is substantially identical to a region comprising 19-25 nucleotides between any one of the nucleotide positions selected from:
a) 52 to 93;
b) 133 to 184;
c) 246 to 304;
d) 337 to 414;
e) 433 to 503;
f) 522 to 571;
g) 617 to 700;
h) 717 to 737;
i) 836 to 905;
j) 944 to 964;
k) 1013 to 1093;
l) 1353 to 1386; and
m) 1407 to 1427,
from the 5′ end of an Angiopoietin-like protein 3 (ANGPTL-3) mRNA sequence according to SEQ ID NO: 1,
and the anti-sense strand is substantially complementary to the sense strand such that the sense strand and the anti-sense strand together form a double stranded region.

2.-51. (canceled)


SEQUENCE LISTING (635 sequences)

SEQ ID NO: 1 - DNA, 2926 bp, Homo sapiens
  source 1..2926: mol_type = genomic DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 2 - DNA, 22 bp, Homo sapiens
  source 1..22: mol_type = genomic DNA
tcaataaaaagaaggagcttaa

SEQ ID NO: 3 - DNA, 22 bp, Homo sapiens
  source 1..22: mol_type = genomic DNA
taaatttttacatcgtctaaca

SEQ ID NO: 4 - DNA, 22 bp, Homo sapiens
  source 1..22: mol_type = genomic DNA
tataaaaagactgatcaaatat

SEQ ID NO: 5 - DNA, 22 bp, Homo sapiens
  source 1..22: mol_type = genomic DNA
tgacatattctttacctcttca

SEQ ID NO: 6 - DNA, 22 bp, Homo sapiens
  source 1..22: mol_type = genomic DNA
ttttaagtgaagttacttctgg

SEQ ID NO: 7 - DNA, 22 bp, Homo sapiens
  source 1..22: mol_type = genomic DNA
tttttaagtgaagttacttctg

SEQ ID NO: 8 - DNA, 22 bp, Homo sapiens
  source 1..22: mol_type = genomic DNA
tgaagatagagaaatttctgtg

SEQ ID NO: 9 - DNA, 22 bp, Homo sapiens
  source 1..22: mol_type = genomic DNA
tgaaaaacttgagagttgctgg

SEQ ID NO: 10 - DNA, 22 bp, Homo sapiens
  source 1..22: mol_type = genomic DNA
ttaatgtttgttgtctttccag

SEQ ID NO: 11 - DNA, 22 bp, Homo sapiens
  source 1..22: mol_type = genomic DNA
tgtataaccttccattttgaga

SEQ ID NO: 12 - DNA, 22 bp, Homo sapiens
  source 1..22: mol_type = genomic DNA
ttttatagagtataaccttcca

SEQ ID NO: 13 - DNA, 22 bp, Homo sapiens
  source 1..22: mol_type = genomic DNA
tttgattttatagagtataacc

SEQ ID NO: 14 - DNA, 22 bp, Homo sapiens
  source 1..22: mol_type = genomic DNA
taaagaaggagcttaattgtga

SEQ ID NO: 15 - DNA, 22 bp, Homo sapiens
  source 1..22: mol_type = genomic DNA
tataaaaagaaggagcttaatt

SEQ ID NO: 16 - DNA, 22 bp, Homo sapiens
  source 1..22: mol_type = genomic DNA
taataaaaagaaggagcttaat

SEQ ID NO: 17 - DNA, 22 bp, Homo sapiens
  source 1..22: mol_type = genomic DNA
taaataactagaggaacaataa

SEQ ID NO: 18 - DNA, 22 bp, Homo sapiens
  source 1..22: mol_type = genomic DNA
taaatcttgattttggctctgg

SEQ ID NO: 19 - DNA, 22 bp, Homo sapiens
  source 1..22: mol_type = genomic DNA
tcatagcaaatcttgattttgg

SEQ ID NO: 20 - DNA, 22 bp, Homo sapiens
  source 1..22: mol_type = genomic DNA
tttgattttggctctggagata

SEQ ID NO: 21 - DNA, 22 bp, Homo sapiens
  source 1..22: mol_type = genomic DNA
tatcgtctaacatagcaaatct

SEQ ID NO: 22 - DNA, 22 bp, Homo sapiens
  source 1..22: mol_type = genomic DNA
tatttttacatcgtctaacata

SEQ ID NO: 23 - DNA, 22 bp, Homo sapiens
  source 1..22: mol_type = genomic DNA
taaaatttttacatcgtctaac

SEQ ID NO: 24 - DNA, 22 bp, Homo sapiens
  source 1..22: mol_type = genomic DNA
tgatagatcataaaaagactga

SEQ ID NO: 25 - DNA, 22 bp, Homo sapiens
  source 1..22: mol_type = genomic DNA
ttaaaaagactgatcaaatatg

SEQ ID NO: 26 - DNA, 22 bp, Homo sapiens
  source 1..22: mol_type = genomic DNA
tatagatcataaaaagactgat

SEQ ID NO: 27 - DNA, 22 bp, Homo sapiens
  source 1..22: mol_type = genomic DNA
ttagtttatatgtagttcttct

SEQ ID NO: 28 - DNA, 22 bp, Homo sapiens
  source 1..22: mol_type = genomic DNA
ttttatatgtagttcttctcag

SEQ ID NO: 29 - DNA, 22 bp, Homo sapiens
  source 1..22: mol_type = genomic DNA
tatttttgacttgtagtttata

SEQ ID NO: 30 - DNA, 22 bp, Homo sapiens
  source 1..22: mol_type = genomic DNA
tttaagttagttagttgctctt

SEQ ID NO: 31 - DNA, 22 bp, Homo sapiens
  source 1..22: mol_type = genomic DNA
tattaagttagttagttgctct

SEQ ID NO: 32 - DNA, 22 bp, Homo sapiens
  source 1..22: mol_type = genomic DNA
tttgattttgaattaagttagt

SEQ ID NO: 33 - DNA, 22 bp, Homo sapiens
  source 1..22: mol_type = genomic DNA
tttctaaatatttcactttttg

SEQ ID NO: 34 - DNA, 22 bp, Homo sapiens
  source 1..22: mol_type = genomic DNA
tttagttagttgctcttctaaa

SEQ ID NO: 35 - DNA, 22 bp, Homo sapiens
  source 1..22: mol_type = genomic DNA
tctattatcttgtttttctaca

SEQ ID NO: 36 - DNA, 22 bp, Homo sapiens
  source 1..22: mol_type = genomic DNA
tatgctattatcttgtttttct

SEQ ID NO: 37 - DNA, 22 bp, Homo sapiens
  source 1..22: mol_type = genomic DNA
taagatagagaaatttctgtgg

SEQ ID NO: 38 - DNA, 22 bp, Homo sapiens
  source 1..22: mol_type = genomic DNA
tgagaaatttctgtgggttctt

SEQ ID NO: 39 - DNA, 22 bp, Homo sapiens
  source 1..22: mol_type = genomic DNA
tctgaagaaagggagtagttct

SEQ ID NO: 40 - DNA, 22 bp, Homo sapiens
  source 1..22: mol_type = genomic DNA
taaaacttgagagttgctgggt

SEQ ID NO: 41 - DNA, 22 bp, Homo sapiens
  source 1..22: mol_type = genomic DNA
tttgaattaatgtccatggact

SEQ ID NO: 42 - DNA, 22 bp, Homo sapiens
  source 1..22: mol_type = genomic DNA
taaaccatatttgtagttctcc

SEQ ID NO: 43 - DNA, 22 bp, Homo sapiens
  source 1..22: mol_type = genomic DNA
ttaattagattgcttcactatg

SEQ ID NO: 44 - DNA, 22 bp, Homo sapiens
  source 1..22: mol_type = genomic DNA
ttataatgtttgttgtctttcc

SEQ ID NO: 45 - DNA, 22 bp, Homo sapiens
  source 1..22: mol_type = genomic DNA
tatataatgtttgttgtctttc

SEQ ID NO: 46 - DNA, 22 bp, Homo sapiens
  source 1..22: mol_type = genomic DNA
taaagaatattcaatataatgt

SEQ ID NO: 47 - DNA, 22 bp, Homo sapiens
  source 1..22: mol_type = genomic DNA
taaaaagaaggagcttaattgt

SEQ ID NO: 48 - DNA, 22 bp, Homo sapiens
  source 1..22: mol_type = genomic DNA
ttttacatcgtctaacatagca

SEQ ID NO: 49 - DNA, 22 bp, Homo sapiens
  source 1..22: mol_type = genomic DNA
tttttacatcgtctaacatagc

SEQ ID NO: 50 - DNA, 22 bp, Homo sapiens
  source 1..22: mol_type = genomic DNA
tctaacatagcaaatcttgatt

SEQ ID NO: 51 - DNA, 22 bp, Homo sapiens
  source 1..22: mol_type = genomic DNA
tttgaaatatgtcattaatttg

SEQ ID NO: 52 - DNA, 22 bp, Homo sapiens
  source 1..22: mol_type = genomic DNA
tcaaatatgttgagtttttgaa

SEQ ID NO: 53 - DNA, 22 bp, Homo sapiens
  source 1..22: mol_type = genomic DNA
tatgtagttcttctcagttcct

SEQ ID NO: 54 - DNA, 22 bp, Homo sapiens
  source 1..22: mol_type = genomic DNA
tttatatgtagttcttctcagt

SEQ ID NO: 55 - DNA, 22 bp, Homo sapiens
  source 1..22: mol_type = genomic DNA
tcaagtgacatattctttacct

SEQ ID NO: 56 - DNA, 22 bp, Homo sapiens
  source 1..22: mol_type = genomic DNA
tttgagttgagttcaagtgaca

SEQ ID NO: 57 - DNA, 22 bp, Homo sapiens
  source 1..22: mol_type = genomic DNA
taagttagttagttgctcttct

SEQ ID NO: 58 - DNA, 22 bp, Homo sapiens
  source 1..22: mol_type = genomic DNA
tgaattaagttagttagttgct

SEQ ID NO: 59 - DNA, 22 bp, Homo sapiens
  source 1..22: mol_type = genomic DNA
tgttgaagtagaattttttctt

SEQ ID NO: 60 - DNA, 22 bp, Homo sapiens
  source 1..22: mol_type = genomic DNA
tttgttgaagtagaattttttc

SEQ ID NO: 61 - DNA, 22 bp, Homo sapiens
  source 1..22: mol_type = genomic DNA
tttcactttttgttgaagtaga

SEQ ID NO: 62 - DNA, 22 bp, Homo sapiens
  source 1..22: mol_type = genomic DNA
ttttatttgactatgctgttgg

SEQ ID NO: 63 - DNA, 20 bp, Homo sapiens
  source 1..20: mol_type = genomic DNA
aagctccttctttttattga

SEQ ID NO: 64 - DNA, 20 bp, Homo sapiens
  source 1..20: mol_type = genomic DNA
ttagacgatgtaaaaattta

SEQ ID NO: 65 - DNA, 20 bp, Homo sapiens
  source 1..20: mol_type = genomic DNA
atttgatcagtctttttata

SEQ ID NO: 66 - DNA, 20 bp, Homo sapiens
  source 1..20: mol_type = genomic DNA
aagaggtaaagaatatgtca

SEQ ID NO: 67 - DNA, 20 bp, Homo sapiens
  source 1..20: mol_type = genomic DNA
agaagtaacttcacttaaaa

SEQ ID NO: 68 - DNA, 20 bp, Homo sapiens
  source 1..20: mol_type = genomic DNA
gaagtaacttcacttaaaaa

SEQ ID NO: 69 - DNA, 20 bp, Homo sapiens
  source 1..20: mol_type = genomic DNA
cagaaatttctctatcttca

SEQ ID NO: 70 - DNA, 20 bp, Homo sapiens
  source 1..20: mol_type = genomic DNA
agcaactctcaagtttttca

SEQ ID NO: 71 - DNA, 20 bp, Homo sapiens
  source 1..20: mol_type = genomic DNA
ggaaagacaacaaacattaa

SEQ ID NO: 72 - DNA, 20 bp, Homo sapiens
  source 1..20: mol_type = genomic DNA
tcaaaatggaaggttataca

SEQ ID NO: 73 - DNA, 20 bp, Homo sapiens
  source 1..20: mol_type = genomic DNA
gaaggttatactctataaaa

SEQ ID NO: 74 - DNA, 20 bp, Homo sapiens
  source 1..20: mol_type = genomic DNA
ttatactctataaaatcaaa

SEQ ID NO: 75 - DNA, 20 bp, Homo sapiens
  source 1..20: mol_type = genomic DNA
acaattaagctccttcttta

SEQ ID NO: 76 - DNA, 20 bp, Homo sapiens
  source 1..20: mol_type = genomic DNA
ttaagctccttctttttata

SEQ ID NO: 77 - DNA, 20 bp, Homo sapiens
  source 1..20: mol_type = genomic DNA
taagctccttctttttatta

SEQ ID NO: 78 - DNA, 20 bp, Homo sapiens
  source 1..20: mol_type = genomic DNA
attgttcctctagttattta

SEQ ID NO: 79 - DNA, 20 bp, Homo sapiens
  source 1..20: mol_type = genomic DNA
agagccaaaatcaagattta

SEQ ID NO: 80 - DNA, 20 bp, Homo sapiens
  source 1..20: mol_type = genomic DNA
aaaatcaagatttgctatga

SEQ ID NO: 81 - DNA, 20 bp, Homo sapiens
  source 1..20: mol_type = genomic DNA
tctccagagccaaaatcaaa

SEQ ID NO: 82 - DNA, 20 bp, Homo sapiens
  source 1..20: mol_type = genomic DNA
atttgctatgttagacgata

SEQ ID NO: 83 - DNA, 20 bp, Homo sapiens
  source 1..20: mol_type = genomic DNA
tgttagacgatgtaaaaata

SEQ ID NO: 84 - DNA, 20 bp, Homo sapiens
  source 1..20: mol_type = genomic DNA
tagacgatgtaaaaatttta

SEQ ID NO: 85 - DNA, 20 bp, Homo sapiens
  source 1..20: mol_type = genomic DNA
agtctttttatgatctatca

SEQ ID NO: 86 - DNA, 20 bp, Homo sapiens
  source 1..20: mol_type = genomic DNA
tatttgatcagtctttttaa

SEQ ID NO: 87 - DNA, 20 bp, Homo sapiens
  source 1..20: mol_type = genomic DNA
cagtctttttatgatctata

SEQ ID NO: 88 - DNA, 20 bp, Homo sapiens
  source 1..20: mol_type = genomic DNA
aagaactacatataaactaa

SEQ ID NO: 89 - DNA, 20 bp, Homo sapiens
  source 1..20: mol_type = genomic DNA
gagaagaactacatataaaa

SEQ ID NO: 90 - DNA, 20 bp, Homo sapiens
  source 1..20: mol_type = genomic DNA
taaactacaagtcaaaaata

SEQ ID NO: 91 - DNA, 20 bp, Homo sapiens
  source 1..20: mol_type = genomic DNA
gagcaactaactaacttaaa

SEQ ID NO: 92 - DNA, 20 bp, Homo sapiens
  source 1..20: mol_type = genomic DNA
agcaactaactaacttaata

SEQ ID NO: 93 - DNA, 20 bp, Homo sapiens
  source 1..20: mol_type = genomic DNA
taacttaattcaaaatcaaa

SEQ ID NO: 94 - DNA, 20 bp, Homo sapiens
  source 1..20: mol_type = genomic DNA
aaaagtgaaatatttagaaa

SEQ ID NO: 95 - DNA, 20 bp, Homo sapiens
  source 1..20: mol_type = genomic DNA
tagaagagcaactaactaaa

SEQ ID NO: 96 - DNA, 20 bp, Homo sapiens
  source 1..20: mol_type = genomic DNA
tagaaaaacaagataataga

SEQ ID NO: 97 - DNA, 20 bp, Homo sapiens
  source 1..20: mol_type = genomic DNA
aaaaacaagataatagcata

SEQ ID NO: 98 - DNA, 20 bp, Homo sapiens
  source 1..20: mol_type = genomic DNA
acagaaatttctctatctta

SEQ ID NO: 99 - DNA, 20 bp, Homo sapiens
  source 1..20: mol_type = genomic DNA
gaacccacagaaatttctca

SEQ ID NO: 100 - DNA, 20 bp, Homo sapiens
  source 1..20: mol_type = genomic DNA
aactactccctttcttcaga

SEQ ID NO: 101 - DNA, 20 bp, Homo sapiens
  source 1..20: mol_type = genomic DNA
ccagcaactctcaagtttta

SEQ ID NO: 102 - DNA, 20 bp, Homo sapiens
  source 1..20: mol_type = genomic DNA
tccatggacattaattcaaa

SEQ ID NO: 103 - DNA, 20 bp, Homo sapiens
  source 1..20: mol_type = genomic DNA
agaactacaaatatggttta

SEQ ID NO: 104 - DNA, 20 bp, Homo sapiens
  source 1..20: mol_type = genomic DNA
tagtgaagcaatctaattaa

SEQ ID NO: 105 - DNA, 20 bp, Homo sapiens
  source 1..20: mol_type = genomic DNA
aaagacaacaaacattataa

SEQ ID NO: 106 - DNA, 20 bp, Homo sapiens
  source 1..20: mol_type = genomic DNA
aagacaacaaacattatata

SEQ ID NO: 107 - DNA, 20 bp, Homo sapiens
  source 1..20: mol_type = genomic DNA
attatattgaatattcttta

SEQ ID NO: 108 - DNA, 20 bp, Homo sapiens
  source 1..20: mol_type = genomic DNA
aattaagctccttcttttta

SEQ ID NO: 109 - DNA, 20 bp, Homo sapiens
  source 1..20: mol_type = genomic DNA
ctatgttagacgatgtaaaa

SEQ ID NO: 110 - DNA, 20 bp, Homo sapiens
  source 1..20: mol_type = genomic DNA
tatgttagacgatgtaaaaa

SEQ ID NO: 111 - DNA, 20 bp, Homo sapiens
  source 1..20: mol_type = genomic DNA
tcaagatttgctatgttaga

SEQ ID NO: 112 - DNA, 20 bp, Homo sapiens
  source 1..20: mol_type = genomic DNA
aattaatgacatatttcaaa

SEQ ID NO: 113 - DNA, 20 bp, Homo sapiens
  source 1..20: mol_type = genomic DNA
caaaaactcaacatatttga

SEQ ID NO: 114 - DNA, 20 bp, Homo sapiens
  source 1..20: mol_type = genomic DNA
gaactgagaagaactacata

SEQ ID NO: 115 - DNA, 20 bp, Homo sapiens
  source 1..20: mol_type = genomic DNA
tgagaagaactacatataaa

SEQ ID NO: 116 - DNA, 20 bp, Homo sapiens
  source 1..20: mol_type = genomic DNA
gtaaagaatatgtcacttga

SEQ ID NO: 117 - DNA, 20 bp, Homo sapiens
  source 1..20: mol_type = genomic DNA
tcacttgaactcaactcaaa

SEQ ID NO: 118 - DNA, 20 bp, Homo sapiens
  source 1..20: mol_type = genomic DNA
aagagcaactaactaactta

SEQ ID NO: 119 - DNA, 20 bp, Homo sapiens
  source 1..20: mol_type = genomic DNA
caactaactaacttaattca

SEQ ID NO: 120 - DNA, 20 bp, Homo sapiens
  source 1..20: mol_type = genomic DNA
gaaaaaattctacttcaaca

SEQ ID NO: 121 - DNA, 20 bp, Homo sapiens
  source 1..20: mol_type = genomic DNA
aaaaattctacttcaacaaa

SEQ ID NO: 122 - DNA, 20 bp, Homo sapiens
  source 1..20: mol_type = genomic DNA
tacttcaacaaaaagtgaaa

SEQ ID NO: 123 - DNA, 20 bp, Homo sapiens
  source 1..20: mol_type = genomic DNA
aacagcatagtcaaataaaa

SEQ ID NO: 124 - DNA, 20 bp, Homo sapiens
  source 1..20: mol_type = genomic DNA
agttccacgttgcttgaaaa

SEQ ID NO: 125 - DNA, 20 bp, Homo sapiens
  source 1..20: mol_type = genomic DNA
ttccacgttgcttgaaatta

SEQ ID NO: 126 - DNA, 20 bp, Homo sapiens
  source 1..20: mol_type = genomic DNA
cacgttgcttgaaattgaaa

SEQ ID NO: 127 - DNA, 20 bp, Homo sapiens
  source 1..20: mol_type = genomic DNA
acgttgcttgaaattgaaaa

SEQ ID NO: 128 - DNA, 20 bp, Homo sapiens
  source 1..20: mol_type = genomic DNA
gttgcttgaaattgaaaata

SEQ ID NO: 129 - DNA, 20 bp, Homo sapiens
  source 1..20: mol_type = genomic DNA
ttgcttgaaattgaaaatca

SEQ ID NO: 130 - DNA, 20 bp, Homo sapiens
  source 1..20: mol_type = genomic DNA
ctccttctttttattgttca

SEQ ID NO: 131 - DNA, 20 bp, Homo sapiens
  source 1..20: mol_type = genomic DNA
tagttatttcctccagaata

SEQ ID NO: 132 - DNA, 20 bp, Homo sapiens
  source 1..20: mol_type = genomic DNA
acaattcatcatttgattca

SEQ ID NO: 133 - DNA, 20 bp, Homo sapiens
  source 1..20: mol_type = genomic DNA
cagagccaaaatcaagatta

SEQ ID NO: 134 - DNA, 20 bp, Homo sapiens
  source 1..20: mol_type = genomic DNA
ccaaaatcaagatttgctaa

SEQ ID NO: 135 - DNA, 20 bp, Homo sapiens
  source 1..20: mol_type = genomic DNA
caaaatcaagatttgctata

SEQ ID NO: 136 - DNA, 20 bp, Homo sapiens
  source 1..20: mol_type = genomic DNA
aagatttgctatgttagaca

SEQ ID NO: 137 - DNA, 20 bp, Homo sapiens
  source 1..20: mol_type = genomic DNA
gacgatgtaaaaattttaga

SEQ ID NO: 138 - DNA, 20 bp, Homo sapiens
  source 1..20: mol_type = genomic DNA
ccaatggcctccttcagtta

SEQ ID NO: 139 - DNA, 20 bp, Homo sapiens
  source 1..20: mol_type = genomic DNA
caatggcctccttcagttga

SEQ ID NO: 140 - DNA, 20 bp, Homo sapiens
  source 1..20: mol_type = genomic DNA
cttcagttgggacatggtca

SEQ ID NO: 141 - DNA, 20 bp, Homo sapiens
  source 1..20: mol_type = genomic DNA
agttgggacatggtcttaaa

SEQ ID NO: 142 - DNA, 20 bp, Homo sapiens
  source 1..20: mol_type = genomic DNA
ttgggacatggtcttaaaga

SEQ ID NO: 143 - DNA, 20 bp, Homo sapiens
  source 1..20: mol_type = genomic DNA
gacatggtcttaaagactta

SEQ ID NO: 144 - DNA, 20 bp, Homo sapiens
  source 1..20: mol_type = genomic DNA
acatggtcttaaagacttta

SEQ ID NO: 145 - DNA, 20 bp, Homo sapiens
  source 1..20: mol_type = genomic DNA
tcttaaagactttgtccata

SEQ ID NO: 146 - DNA, 20 bp, Homo sapiens
  source 1..20: mol_type = genomic DNA
ttaaagactttgtccataaa

SEQ ID NO: 147 - DNA, 20 bp, Homo sapiens
  source 1..20: mol_type = genomic DNA
ggccaaattaatgacatata

SEQ ID NO: 148 - DNA, 20 bp, Homo sapiens
  source 1..20: mol_type = genomic DNA
gccaaattaatgacatatta

SEQ ID NO: 149 - DNA, 20 bp, Homo sapiens
  source 1..20: mol_type = genomic DNA
ccaaattaatgacatattta

SEQ ID NO: 150 - DNA, 20 bp, Homo sapiens
  source 1..20: mol_type = genomic DNA
caaattaatgacatatttca

SEQ ID NO: 151 - DNA, 20 bp, Homo sapiens
  source 1..20: mol_type = genomic DNA
acatatttcaaaaactcaaa

SEQ ID NO: 152 - DNA, 20 bp, Homo sapiens
  source 1..20: mol_type = genomic DNA
tcaacatatttgatcagtca

SEQ ID NO: 153 - DNA, 20 bp, Homo sapiens
  source 1..20: mol_type = genomic DNA
atatttgatcagtcttttta

SEQ ID NO: 154 - DNA, 20 bp, Homo sapiens
  source 1..20: mol_type = genomic DNA
caaaccagtgaaatcaaaga

SEQ ID NO: 155 - DNA, 20 bp, Homo sapiens
  source 1..20: mol_type = genomic DNA
cagtgaaatcaaagaagaaa

SEQ ID NO: 156 - DNA, 20 bp, Homo sapiens
  source 1..20: mol_type = genomic DNA
agtgaaatcaaagaagaaga

SEQ ID NO: 157 - DNA, 20 bp, Homo sapiens
  source 1..20: mol_type = genomic DNA
tgaaatcaaagaagaagaaa

SEQ ID NO: 158 - DNA, 20 bp, Homo sapiens
  source 1..20: mol_type = genomic DNA
aaatcaaagaagaagaaaaa

SEQ ID NO: 159 - DNA, 20 bp, Homo sapiens
  source 1..20: mol_type = genomic DNA
aaagaagaagaaaaggaaca

SEQ ID NO: 160 - DNA, 20 bp, Homo sapiens
  source 1..20: mol_type = genomic DNA
aagaagaaaaggaactgaga

SEQ ID NO: 161 - DNA, 20 bp, Homo sapiens
  source 1..20: mol_type = genomic DNA
aagaaaaggaactgagaaga

SEQ ID NO: 162 - DNA, 20 bp, Homo sapiens
  source 1..20: mol_type = genomic DNA
aaaaggaactgagaagaaca

SEQ ID NO: 163 - DNA, 20 bp, Homo sapiens
  source 1..20: mol_type = genomic DNA
aactgagaagaactacataa

SEQ ID NO: 164 - DNA, 20 bp, Homo sapiens
  source 1..20: mol_type = genomic DNA
agaagaactacatataaaca

SEQ ID NO: 165 - DNA, 20 bp, Homo sapiens
  source 1..20: mol_type = genomic DNA
aactacatataaactacaaa

SEQ ID NO: 166 - DNA, 20 bp, Homo sapiens
  source 1..20: mol_type = genomic DNA
atataaactacaagtcaaaa

SEQ ID NO: 167 - DNA, 20 bp, Homo sapiens
  source 1..20: mol_type = genomic DNA
tcaaaaatgaagaggtaaaa

SEQ ID NO: 168 - DNA, 20 bp, Homo sapiens
  source 1..20: mol_type = genomic DNA
acttgaactcaactcaaaaa

SEQ ID NO: 169 - DNA, 20 bp, Homo sapiens
  source 1..20: mol_type = genomic DNA
tcaactcaaaacttgaaaga

SEQ ID NO: 170 - DNA, 20 bp, Homo sapiens
  source 1..20: mol_type = genomic DNA
aaagcctcctagaagaaaaa

SEQ ID NO: 171 - DNA, 20 bp, Homo sapiens
  source 1..20: mol_type = genomic DNA
gcctcctagaagaaaaaata

SEQ ID NO: 172 - DNA, 20 bp, Homo sapiens
  source 1..20: mol_type = genomic DNA
cctcctagaagaaaaaatta

SEQ ID NO: 173 - DNA, 20 bp, Homo sapiens
  source 1..20: mol_type = genomic DNA
ctcctagaagaaaaaattca

SEQ ID NO: 174 - DNA, 20 bp, Homo sapiens
  source 1..20: mol_type = genomic DNA
ctagaagaaaaaattctaca

SEQ ID NO: 175 - DNA, 20 bp, Homo sapiens
  source 1..20: mol_type = genomic DNA
ttcaacaaaaagtgaaataa

SEQ ID NO: 176 - DNA, 20 bp, Homo sapiens
  source 1..20: mol_type = genomic DNA
aacaaaaagtgaaatattta

SEQ ID NO: 177 - DNA, 20 bp, Homo sapiens
  source 1..20: mol_type = genomic DNA
acaaaaagtgaaatatttaa

SEQ ID NO: 178 - DNA, 20 bp, Homo sapiens
  source 1..20: mol_type = genomic DNA
caaaaagtgaaatatttaga

SEQ ID NO: 179 - DNA, 20 bp, Homo sapiens
  source 1..20: mol_type = genomic DNA
aaatatttagaagagcaaca

SEQ ID NO: 180 - DNA, 20 bp, Homo sapiens
  source 1..20: mol_type = genomic DNA
taactaacttaattcaaaaa

SEQ ID NO: 181 - DNA, 20 bp, Homo sapiens
  source 1..20: mol_type = genomic DNA
caagataatagcatcaaaga

SEQ ID NO: 182 - DNA, 20 bp, Homo sapiens
  source 1..20: mol_type = genomic DNA
catcaaagaccttctccaga

SEQ ID NO: 183 - DNA, 20 bp, Homo sapiens
  source 1..20: mol_type = genomic DNA
accaacagcatagtcaaata

SEQ ID NO: 184 - DNA, 20 bp, Homo sapiens
  source 1..20: mol_type = genomic DNA
caacagcatagtcaaataaa

SEQ ID NO: 185 - DNA, 20 bp, Homo sapiens
  source 1..20: mol_type = genomic DNA
cagcatagtcaaataaaaga

SEQ ID NO: 186 - DNA, 20 bp, Homo sapiens
  source 1..20: mol_type = genomic DNA
catagtcaaataaaagaaaa

SEQ ID NO: 187 - DNA, 20 bp, Homo sapiens
  source 1..20: mol_type = genomic DNA
atagtcaaataaaagaaata

SEQ ID NO: 188 - DNA, 20 bp, Homo sapiens
  source 1..20: mol_type = genomic DNA
tcaaataaaagaaatagaaa

SEQ ID NO: 189 - DNA, 20 bp, Homo sapiens
  source 1..20: mol_type = genomic DNA
aaataaaagaaatagaaaaa

SEQ ID NO: 190 - DNA, 20 bp, Homo sapiens
  source 1..20: mol_type = genomic DNA
aagaacccacagaaatttca

SEQ ID NO: 191 - DNA, 20 bp, Homo sapiens
  source 1..20: mol_type = genomic DNA
ccacagaaatttctctatca

SEQ ID NO: 192 - DNA, 20 bp, Homo sapiens
  source 1..20: mol_type = genomic DNA
aagccaagagcaccaagaaa

SEQ ID NO: 193 - DNA, 20 bp, Homo sapiens
  source 1..20: mol_type = genomic DNA
ccaagagcaccaagaactaa

SEQ ID NO: 194 - DNA, 20 bp, Homo sapiens
  source 1..20: mol_type = genomic DNA
caagagcaccaagaactaca

SEQ ID NO: 195 - DNA, 20 bp, Homo sapiens
  source 1..20: mol_type = genomic DNA
aagaactactccctttctta

SEQ ID NO: 196 - DNA, 20 bp, Homo sapiens
  source 1..20: mol_type = genomic DNA
aatgaaataagaaatgtaaa

SEQ ID NO: 197 - DNA, 20 bp, Homo sapiens
  source 1..20: mol_type = genomic DNA
ccatcagacccagcaactca

SEQ ID NO: 198 - DNA, 20 bp, Homo sapiens
  source 1..20: mol_type = genomic DNA
agacccagcaactctcaaga

SEQ ID NO: 199 - DNA, 20 bp, Homo sapiens
  source 1..20: mol_type = genomic DNA
acccagcaactctcaagtta

SEQ ID NO: 200 - DNA, 22 bp, Homo sapiens
  source 1..22: mol_type = genomic DNA
tttcaaagctttctgaatctgt

SEQ ID NO: 201 - DNA, 20 bp, Homo sapiens
  source 1..20: mol_type = genomic DNA
caactctcaagtttttcata

SEQ ID NO: 202 - DNA, 20 bp, Homo sapiens
  source 1..20: mol_type = genomic DNA
tcaagtttttcatgtctaca

SEQ ID NO: 203 - DNA, 20 bp, Homo sapiens
  source 1..20: mol_type = genomic DNA
aggtagtccatggacattaa

SEQ ID NO: 204 - DNA, 20 bp, Homo sapiens
  source 1..20: mol_type = genomic DNA
gtagtccatggacattaata

SEQ ID NO: 205 - DNA, 20 bp, Homo sapiens
  source 1..20: mol_type = genomic DNA
tagtccatggacattaatta

SEQ ID NO: 206 - DNA, 20 bp, Homo sapiens
  source 1..20: mol_type = genomic DNA
atggacattaattcaacata

SEQ ID NO: 207 - DNA, 20 bp, Homo sapiens
  source 1..20: mol_type = genomic DNA
attaattcaacatcgaataa

SEQ ID NO: 208 - DNA, 20 bp, Homo sapiens
  source 1..20: mol_type = genomic DNA
gaatagatggatcacaaaaa

SEQ ID NO: 209 - DNA, 20 bp, Homo sapiens
  source 1..20: mol_type = genomic DNA
aatagatggatcacaaaaca

SEQ ID NO: 210 - DNA, 20 bp, Homo sapiens
  source 1..20: mol_type = genomic DNA
atggatcacaaaacttcaaa

SEQ ID NO: 211 - DNA, 20 bp, Homo sapiens
  source 1..20: mol_type = genomic DNA
aaacgtgggagaactacaaa

SEQ ID NO: 212 - DNA, 20 bp, Homo sapiens
  source 1..20: mol_type = genomic DNA
gtgggagaactacaaatata

SEQ ID NO: 213 - DNA, 20 bp, Homo sapiens
  source 1..20: mol_type = genomic DNA
tgggagaactacaaatatga

SEQ ID NO: 214 - DNA, 20 bp, Homo sapiens
  source 1..20: mol_type = genomic DNA
gagaactacaaatatggtta

SEQ ID NO: 215 - DNA, 20 bp, Homo sapiens
  source 1..20: mol_type = genomic DNA
gttgggcctagagaagataa

SEQ ID NO: 216 - DNA, 20 bp, Homo sapiens
  source 1..20: mol_type = genomic DNA
ttgggcctagagaagatata

SEQ ID NO: 217 - DNA, 20 bp, Homo sapiens
  source 1..20: mol_type = genomic DNA
gcctagagaagatatactca

SEQ ID NO: 218 - DNA, 20 bp, Homo sapiens
  source 1..20: mol_type = genomic DNA
tccatagtgaagcaatctaa

SEQ ID NO: 219 - DNA, 20 bp, Homo sapiens
  source 1..20: mol_type = genomic DNA
catagtgaagcaatctaata

SEQ ID NO: 220 - DNA, 20 bp, Homo sapiens
  source 1..20: mol_type = genomic DNA
atagtgaagcaatctaatta

SEQ ID NO: 221 - DNA, 20 bp, Homo sapiens
  source 1..20: mol_type = genomic DNA
gaattgagttggaagactga

SEQ ID NO: 222 - DNA, 20 bp, Homo sapiens
  source 1..20: mol_type = genomic DNA
tggaaagacaacaaacatta

SEQ ID NO: 223 - DNA, 20 bp, Homo sapiens
  source 1..20: mol_type = genomic DNA
gaaagacaacaaacattata

SEQ ID NO: 224 - DNA, 20 bp, Homo sapiens
  source 1..20: mol_type = genomic DNA
gacaacaaacattatattga

SEQ ID NO: 225 - DNA, 20 bp, Homo sapiens
  source 1..20: mol_type = genomic DNA
aacaaacattatattgaata

SEQ ID NO: 226 - DNA, 20 bp, Homo sapiens
  source 1..20: mol_type = genomic DNA
aaacattatattgaatatta

SEQ ID NO: 227 - DNA, 20 bp, Homo sapiens
  source 1..20: mol_type = genomic DNA
gaaatcacgaaaccaactaa

SEQ ID NO: 228 - DNA, 20 bp, Homo sapiens
  source 1..20: mol_type = genomic DNA
aaatcacgaaaccaactata

SEQ ID NO: 229 - DNA, 20 bp, Homo sapiens
  source 1..20: mol_type = genomic DNA
gaaaccaactatacgctaca

SEQ ID NO: 230 - DNA, 20 bp, Homo sapiens
  source 1..20: mol_type = genomic DNA
gcaatgtccccaatgcaata

SEQ ID NO: 231 - DNA, 20 bp, Homo sapiens
  source 1..20: mol_type = genomic DNA
caatgtccccaatgcaatca

SEQ ID NO: 232 - DNA, 20 bp, Homo sapiens
  source 1..20: mol_type = genomic DNA
caatgcaatcccggaaaaca

SEQ ID NO: 233 - DNA, 20 bp, Homo sapiens
  source 1..20: mol_type = genomic DNA
aatcccggaaaacaaagata

SEQ ID NO: 234 - DNA, 20 bp, Homo sapiens
  source 1..20: mol_type = genomic DNA
ggaaaacaaagatttggtga

SEQ ID NO: 235 - DNA, 20 bp, Homo sapiens
  source 1..20: mol_type = genomic DNA
agattcagaaagctttgaaa

SEQ ID NO: 236 - DNA, 20 bp, Homo sapiens
  source 1..20: mol_type = genomic DNA
gtgttttctacttgggatca

SEQ ID NO: 237 - DNA, 20 bp, Homo sapiens
  source 1..20: mol_type = genomic DNA
ttgggatcacaaagcaaaaa

SEQ ID NO: 238 - DNA, 20 bp, Homo sapiens
  source 1..20: mol_type = genomic DNA
gatcacaaagcaaaaggaca

SEQ ID NO: 239 - DNA, 20 bp, Homo sapiens
  source 1..20: mol_type = genomic DNA
ctggtggtggcatgatgaga

SEQ ID NO: 240 - DNA, 20 bp, Homo sapiens
  source 1..20: mol_type = genomic DNA
tggtggcatgatgagtgtga

SEQ ID NO: 241 - DNA, 20 bp, Homo sapiens
  source 1..20: mol_type = genomic DNA
catgatgagtgtggagaaaa

SEQ ID NO: 242 - DNA, 20 bp, Homo sapiens
  source 1..20: mol_type = genomic DNA
atgatgagtgtggagaaaaa

SEQ ID NO: 243 - DNA, 20 bp, Homo sapiens
  source 1..20: mol_type = genomic DNA
tgtggagaaaacaacctaaa

SEQ ID NO: 244 - DNA, 20 bp, Homo sapiens
  source 1..20: mol_type = genomic DNA
tggagaaaacaacctaaata

SEQ ID NO: 245 - DNA, 20 bp, Homo sapiens
  source 1..20: mol_type = genomic DNA
aacctaaatggtaaatataa

SEQ ID NO: 246 - DNA, 20 bp, Homo sapiens
  source 1..20: mol_type = genomic DNA
acctaaatggtaaatataaa

SEQ ID NO: 247 - DNA, 20 bp, Homo sapiens
  source 1..20: mol_type = genomic DNA
cctaaatggtaaatataaca

SEQ ID NO: 248 - DNA, 20 bp, Homo sapiens
  source 1..20: mol_type = genomic DNA
taaatggtaaatataacaaa

SEQ ID NO: 249 - DNA, 20 bp, Homo sapiens
  source 1..20: mol_type = genomic DNA
gtaaatataacaaaccaaga

SEQ ID NO: 250 - DNA, 20 bp, Homo sapiens
  source 1..20: mol_type = genomic DNA
tctcaaaatggaaggttata

SEQ ID NO: 251 - DNA, 20 bp, Homo sapiens
  source 1..20: mol_type = genomic DNA
aaggttatactctataaaaa

SEQ ID NO: 252 - DNA, 20 bp, Homo sapiens
  source 1..20: mol_type = genomic DNA
ggttatactctataaaatca

SEQ ID NO: 253 - DNA, 20 bp, Homo sapiens
  source 1..20: mol_type = genomic DNA
tctataaaatcaaccaaaaa

SEQ ID NO: 254 - DNA, 20 bp, Homo sapiens
  source 1..20: mol_type = genomic DNA
tataaaatcaaccaaaatga

SEQ ID NO: 255 - DNA, 20 bp, Homo sapiens
  source 1..20: mol_type = genomic DNA
aaccaaaatgttgatccata

SEQ ID NO: 256 - DNA, 20 bp, Homo sapiens
  source 1..20: mol_type = genomic DNA
atccatccaacagattcaga

SEQ ID NO: 257 - DNA, 20 bp, Homo sapiens
  source 1..20: mol_type = genomic DNA
catccaacagattcagaaaa

SEQ ID NO: 258 - DNA, 20 bp, Homo sapiens
  source 1..20: mol_type = genomic DNA
atccaacagattcagaaaga

SEQ ID NO: 259 - DNA, 20 bp, Homo sapiens
  source 1..20: mol_type = genomic DNA
caacagattcagaaagctta

SEQ ID NO: 260 - DNA, 20 bp, Homo sapiens
  source 1..20: mol_type = genomic DNA
attcagaaagctttgaatga

SEQ ID NO: 261 - DNA, 20 bp, Homo sapiens
  source 1..20: mol_type = genomic DNA
aatgaactgaggcaaattta

SEQ ID NO: 262 - DNA, 20 bp, Homo sapiens
  source 1..20: mol_type = genomic DNA
aactgaggcaaatttaaaaa

SEQ ID NO: 263 - DNA, 20 bp, Homo sapiens
  source 1..20: mol_type = genomic DNA
actgaggcaaatttaaaaga

SEQ ID NO: 264 - DNA, 20 bp, Homo sapiens
  source 1..20: mol_type = genomic DNA
aggcaaatttaaaaggcaaa

SEQ ID NO: 265 - DNA, 20 bp, Homo sapiens
  source 1..20: mol_type = genomic DNA
gcaaatttaaaaggcaataa

SEQ ID NO: 266 - DNA, 20 bp, Homo sapiens
  source 1..20: mol_type = genomic DNA
caaatttaaaaggcaataaa

SEQ ID NO: 267 - DNA, 20 bp, Homo sapiens
  source 1..20: mol_type = genomic DNA
tattaaatccttaagagaaa

SEQ ID NO: 268 - DNA, 22 bp, Homo sapiens
  source 1..22: mol_type = genomic DNA
ttttcaagcaacgtggaactgt

SEQ ID NO: 269 - DNA, 22 bp, Homo sapiens
  source 1..22: mol_type = genomic DNA
taatttcaagcaacgtggaact

SEQ ID NO: 270 - DNA, 22 bp, Homo sapiens
  source 1..22: mol_type = genomic DNA
tttcaatttcaagcaacgtgga

SEQ ID NO: 271 - DNA, 22 bp, Homo sapiens
  source 1..22: mol_type = genomic DNA
ttttcaatttcaagcaacgtgg

SEQ ID NO: 272 - DNA, 22 bp, Homo sapiens
  source 1..22: mol_type = genomic DNA
tattttcaatttcaagcaacgt

SEQ ID NO: 273 - DNA, 22 bp, Homo sapiens
  source 1..22: mol_type = genomic DNA
tgattttcaatttcaagcaacg

SEQ ID NO: 274 - DNA, 22 bp, Homo sapiens
  source 1..22: mol_type = genomic DNA
tgaacaataaaaagaaggagct

SEQ ID NO: 275 - DNA, 22 bp, Homo sapiens
  source 1..22: mol_type = genomic DNA
tattctggaggaaataactaga

SEQ ID NO: 276 - DNA, 22 bp, Homo sapiens
  source 1..22: mol_type = genomic DNA
tgaatcaaatgatgaattgtct

SEQ ID NO: 277 - DNA, 22 bp, Homo sapiens
  source 1..22: mol_type = genomic DNA
taatcttgattttggctctgga

SEQ ID NO: 278 - DNA, 22 bp, Homo sapiens
  source 1..22: mol_type = genomic DNA
ttagcaaatcttgattttggct

SEQ ID NO: 279 - DNA, 22 bp, Homo sapiens
  source 1..22: mol_type = genomic DNA
tatagcaaatcttgattttggc

SEQ ID NO: 280 - DNA, 22 bp, Homo sapiens
  source 1..22: mol_type = genomic DNA
tgtctaacatagcaaatcttga

SEQ ID NO: 281 - DNA, 22 bp, Homo sapiens
  source 1..22: mol_type = genomic DNA
tctaaaatttttacatcgtcta

SEQ ID NO: 282 - DNA, 22 bp, Homo sapiens
  source 1..22: mol_type = genomic DNA
taactgaaggaggccattggct

SEQ ID NO: 283 - DNA, 22 bp, Homo sapiens
  source 1..22: mol_type = genomic DNA
tcaactgaaggaggccattggc

SEQ ID NO: 284 - DNA, 22 bp, Homo sapiens
  source 1..22: mol_type = genomic DNA
tgaccatgtcccaactgaagga

SEQ ID NO: 285 - DNA, 22 bp, Homo sapiens
  source 1..22: mol_type = genomic DNA
tttaagaccatgtcccaactga

SEQ ID NO: 286 - DNA, 22 bp, Homo sapiens
  source 1..22: mol_type = genomic DNA
tctttaagaccatgtcccaact

SEQ ID NO: 287 - DNA, 22 bp, Homo sapiens
  source 1..22: mol_type = genomic DNA
taagtctttaagaccatgtccc

SEQ ID NO: 288 - DNA, 22 bp, Homo sapiens
  source 1..22: mol_type = genomic DNA
taaagtctttaagaccatgtcc

SEQ ID NO: 289 - DNA, 22 bp, Homo sapiens
  source 1..22: mol_type = genomic DNA
tatggacaaagtctttaagacc

SEQ ID NO: 290 - DNA, 22 bp, Homo sapiens
  source 1..22: mol_type = genomic DNA
tttatggacaaagtctttaaga

SEQ ID NO: 291 - DNA, 22 bp, Homo sapiens
  source 1..22: mol_type = genomic DNA
tatatgtcattaatttggccct

SEQ ID NO: 292 - DNA, 22 bp, Homo sapiens
  source 1..22: mol_type = genomic DNA
taatatgtcattaatttggccc

SEQ ID NO: 293 - DNA, 22 bp, Homo sapiens
  source 1..22: mol_type = genomic DNA
taaatatgtcattaatttggcc

SEQ ID NO: 294 - DNA, 22 bp, Homo sapiens
  source 1..22: mol_type = genomic DNA
tgaaatatgtcattaatttggc

SEQ ID NO: 295 - DNA, 22 bp, Homo sapiens
  source 1..22: mol_type = genomic DNA
tttgagtttttgaaatatgtca

SEQ ID NO: 296 - DNA, 22 bp, Homo sapiens
  source 1..22: mol_type = genomic DNA
tgactgatcaaatatgttgagt

SEQ ID NO: 297 - DNA, 22 bp, Homo sapiens
  source 1..22: mol_type = genomic DNA
taaaaagactgatcaaatatgt

SEQ ID NO: 298 - DNA, 22 bp, Homo sapiens
  source 1..22: mol_type = genomic DNA
tctttgatttcactggtttgca

SEQ ID NO: 299 - DNA, 22 bp, Homo sapiens
  source 1..22: mol_type = genomic DNA
tttcttctttgatttcactggt

SEQ ID NO: 300 - DNA, 22 bp, Homo sapiens
  source 1..22: mol_type = genomic DNA
tcttcttctttgatttcactgg

SEQ ID NO: 301 - DNA, 22 bp, Homo sapiens
  source 1..22: mol_type = genomic DNA
tttcttcttctttgatttcact

SEQ ID NO: 302 - DNA, 22 bp, Homo sapiens
  source 1..22: mol_type = genomic DNA
tttttcttcttctttgatttca

SEQ ID NO: 303 - DNA, 22 bp, Homo sapiens
  source 1..22: mol_type = genomic DNA
tgttccttttcttcttctttga

SEQ ID NO: 304 - DNA, 22 bp, Homo sapiens
  source 1..22: mol_type = genomic DNA
tctcagttccttttcttcttct

SEQ ID NO: 305 - DNA, 22 bp, Homo sapiens
  source 1..22: mol_type = genomic DNA
tcttctcagttccttttcttct

SEQ ID NO: 306 - DNA, 22 bp, Homo sapiens
  source 1..22: mol_type = genomic DNA
tgttcttctcagttccttttct

SEQ ID NO: 307 - DNA, 22 bp, Homo sapiens
  source 1..22: mol_type = genomic DNA
ttatgtagttcttctcagttcc

SEQ ID NO: 308 - DNA, 22 bp, Homo sapiens
  source 1..22: mol_type = genomic DNA
tgtttatatgtagttcttctca

SEQ ID NO: 309 - DNA, 22 bp, Homo sapiens
  source 1..22: mol_type = genomic DNA
tttgtagtttatatgtagttct

SEQ ID NO: 310 - DNA, 22 bp, Homo sapiens
  source 1..22: mol_type = genomic DNA
ttttgacttgtagtttatatgt

SEQ ID NO: 311 - DNA, 22 bp, Homo sapiens
  source 1..22: mol_type = genomic DNA
ttttacctcttcatttttgact

SEQ ID NO: 312 - DNA, 22 bp, Homo sapiens
  source 1..22: mol_type = genomic DNA
tttttgagttgagttcaagtga

SEQ ID NO: 313 - DNA, 22 bp, Homo sapiens
  source 1..22: mol_type = genomic DNA
tctttcaagttttgagttgagt

SEQ ID NO: 314 - DNA, 22 bp, Homo sapiens
  source 1..22: mol_type = genomic DNA
tttttcttctaggaggctttca

SEQ ID NO: 315 - DNA, 22 bp, Homo sapiens
  source 1..22: mol_type = genomic DNA
tattttttcttctaggaggctt

SEQ ID NO: 316 - DNA, 22 bp, Homo sapiens
  source 1..22: mol_type = genomic DNA
taattttttcttctaggaggct

SEQ ID NO: 317 - DNA, 22 bp, Homo sapiens
  source 1..22: mol_type = genomic DNA
tgaattttttcttctaggaggc

SEQ ID NO: 318 - DNA, 22 bp, Homo sapiens
  source 1..22: mol_type = genomic DNA
tgtagaattttttcttctagga

SEQ ID NO: 319 - DNA, 22 bp, Homo sapiens
  source 1..22: mol_type = genomic DNA
ttatttcactttttgttgaagt

SEQ ID NO: 320 - DNA, 22 bp, Homo sapiens
  source 1..22: mol_type = genomic DNA
taaatatttcactttttgttga

SEQ ID NO: 321 - DNA, 22 bp, Homo sapiens
  source 1..22: mol_type = genomic DNA
ttaaatatttcactttttgttg

SEQ ID NO: 322 - DNA, 22 bp, Homo sapiens
  source 1..22: mol_type = genomic DNA
tctaaatatttcactttttgtt

SEQ ID NO: 323 - DNA, 22 bp, Homo sapiens
  source 1..22: mol_type = genomic DNA
tgttgctcttctaaatatttca

SEQ ID NO: 324 - DNA, 22 bp, Homo sapiens
  source 1..22: mol_type = genomic DNA
tttttgaattaagttagttagt

SEQ ID NO: 325 - DNA, 22 bp, Homo sapiens
  source 1..22: mol_type = genomic DNA
tctttgatgctattatcttgtt

SEQ ID NO: 326 - DNA, 22 bp, Homo sapiens
  source 1..22: mol_type = genomic DNA
tctggagaaggtctttgatgct

SEQ ID NO: 327 - DNA, 22 bp, Homo sapiens
  source 1..22: mol_type = genomic DNA
tatttgactatgctgttggttt

SEQ ID NO: 328 - DNA, 22 bp, Homo sapiens
  source 1..22: mol_type = genomic DNA
tttatttgactatgctgttggt

SEQ ID NO: 329 - DNA, 22 bp, Homo sapiens
  source 1..22: mol_type = genomic DNA
tcttttatttgactatgctgtt

SEQ ID NO: 330 - DNA, 22 bp, Homo sapiens
  source 1..22: mol_type = genomic DNA
ttttcttttatttgactatgct

SEQ ID NO: 331 - DNA, 22 bp, Homo sapiens
  source 1..22: mol_type = genomic DNA
tatttcttttatttgactatgc

SEQ ID NO: 332 - DNA, 22 bp, Homo sapiens
  source 1..22: mol_type = genomic DNA
tttctatttcttttatttgact

SEQ ID NO: 333 - DNA, 22 bp, Homo sapiens
  source 1..22: mol_type = genomic DNA
tttttctatttcttttatttga

SEQ ID NO: 334 - DNA, 22 bp, Homo sapiens
  source 1..22: mol_type = genomic DNA
tgaaatttctgtgggttcttga

SEQ ID NO: 335 - DNA, 22 bp, Homo sapiens
  source 1..22: mol_type = genomic DNA
tgatagagaaatttctgtgggt

SEQ ID NO: 336 - DNA, 22 bp, Homo sapiens
  source 1..22: mol_type = genomic DNA
tttcttggtgctcttggcttgg

SEQ ID NO: 337 - DNA, 22 bp, Homo sapiens
  source 1..22: mol_type = genomic DNA
ttagttcttggtgctcttggct

SEQ ID NO: 338 - DNA, 22 bp, Homo sapiens
  source 1..22: mol_type = genomic DNA
tgtagttcttggtgctcttggc

SEQ ID NO: 339 - DNA, 22 bp, Homo sapiens
  source 1..22: mol_type = genomic DNA
taagaaagggagtagttcttgg

SEQ ID NO: 340 - DNA, 22 bp, Homo sapiens
  source 1..22: mol_type = genomic DNA
tttacatttcttatttcattca

SEQ ID NO: 341 - DNA, 22 bp, Homo sapiens
  source 1..22: mol_type = genomic DNA
tgagttgctgggtctgatggca

SEQ ID NO: 342 - DNA, 22 bp, Homo sapiens
  source 1..22: mol_type = genomic DNA
tcttgagagttgctgggtctga

SEQ ID NO: 343 - DNA, 22 bp, Homo sapiens
  source 1..22: mol_type = genomic DNA
taacttgagagttgctgggtct

SEQ ID NO: 344 - DNA, 22 bp, Homo sapiens
  source 1..22: mol_type = genomic DNA
taaaaacttgagagttgctggg

SEQ ID NO: 345 - DNA, 22 bp, Homo sapiens
  source 1..22: mol_type = genomic DNA
tatgaaaaacttgagagttgct

SEQ ID NO: 346 - DNA, 22 bp, Homo sapiens
  source 1..22: mol_type = genomic DNA
tgtagacatgaaaaacttgaga

SEQ ID NO: 347 - DNA, 22 bp, Homo sapiens
  source 1..22: mol_type = genomic DNA
ttaatgtccatggactacctga

SEQ ID NO: 348 - DNA, 22 bp, Homo sapiens
  source 1..22: mol_type = genomic DNA
tattaatgtccatggactacct

SEQ ID NO: 349 - DNA, 22 bp, Homo sapiens
  source 1..22: mol_type = genomic DNA
taattaatgtccatggactacc

SEQ ID NO: 350 - DNA, 22 bp, Homo sapiens
  source 1..22: mol_type = genomic DNA
tatgttgaattaatgtccatgg

SEQ ID NO: 351 - DNA, 22 bp, Homo sapiens
  source 1..22: mol_type = genomic DNA
ttattcgatgttgaattaatgt

SEQ ID NO: 352 - DNA, 22 bp, Homo sapiens
  source 1..22: mol_type = genomic DNA
tttttgtgatccatctattcga

SEQ ID NO: 353 - DNA, 22 bp, Homo sapiens
  source 1..22: mol_type = genomic DNA
tgttttgtgatccatctattcg

SEQ ID NO: 354 - DNA, 22 bp, Homo sapiens
  source 1..22: mol_type = genomic DNA
tttgaagttttgtgatccatct

SEQ ID NO: 355 - DNA, 22 bp, Homo sapiens
  source 1..22: mol_type = genomic DNA
tttgtagttctcccacgtttca

SEQ ID NO: 356 - DNA, 22 bp, Homo sapiens
  source 1..22: mol_type = genomic DNA
tatatttgtagttctcccacgt

SEQ ID NO: 357 - DNA, 22 bp, Homo sapiens
  source 1..22: mol_type = genomic DNA
tcatatttgtagttctcccacg

SEQ ID NO: 358 - DNA, 22 bp, Homo sapiens
  source 1..22: mol_type = genomic DNA
taaccatatttgtagttctccc

SEQ ID NO: 359 - DNA, 22 bp, Homo sapiens
  source 1..22: mol_type = genomic DNA
ttatcttctctaggcccaacca

SEQ ID NO: 360 - DNA, 22 bp, Homo sapiens
  source 1..22: mol_type = genomic DNA
tatatcttctctaggcccaacc

SEQ ID NO: 361 - DNA, 22 bp, Homo sapiens
  source 1..22: mol_type = genomic DNA
tgagtatatcttctctaggccc

SEQ ID NO: 362 - DNA, 22 bp, Homo sapiens
  source 1..22: mol_type = genomic DNA
ttagattgcttcactatggagt

SEQ ID NO: 363 - DNA, 22 bp, Homo sapiens
  source 1..22: mol_type = genomic DNA
tattagattgcttcactatgga

SEQ ID NO: 364 - DNA, 22 bp, Homo sapiens
  source 1..22: mol_type = genomic DNA
taattagattgcttcactatgg

SEQ ID NO: 365 - DNA, 22 bp, Homo sapiens
  source 1..22: mol_type = genomic DNA
tcagtcttccaactcaattcgt

SEQ ID NO: 366 - DNA, 22 bp, Homo sapiens
  source 1..22: mol_type = genomic DNA
taatgtttgttgtctttccagt

SEQ ID NO: 367 - DNA, 22 bp, Homo sapiens
  source 1..22: mol_type = genomic DNA
tataatgtttgttgtctttcca

SEQ ID NO: 368 - DNA, 22 bp, Homo sapiens
  source 1..22: mol_type = genomic DNA
tcaatataatgtttgttgtctt

SEQ ID NO: 369 - DNA, 22 bp, Homo sapiens
  source 1..22: mol_type = genomic DNA
tattcaatataatgtttgttgt

SEQ ID NO: 370 - DNA, 22 bp, Homo sapiens
  source 1..22: mol_type = genomic DNA
taatattcaatataatgtttgt

SEQ ID NO: 371 - DNA, 22 bp, Homo sapiens
  source 1..22: mol_type = genomic DNA
ttagttggtttcgtgatttccc

SEQ ID NO: 372 - DNA, 22 bp, Homo sapiens
  source 1..22: mol_type = genomic DNA
tatagttggtttcgtgatttcc

SEQ ID NO: 373 - DNA, 22 bp, Homo sapiens
  source 1..22: mol_type = genomic DNA
tgtagcgtatagttggtttcgt

SEQ ID NO: 374 - DNA, 22 bp, Homo sapiens
  source 1..22: mol_type = genomic DNA
tattgcattggggacattgcca

SEQ ID NO: 375 - DNA, 22 bp, Homo sapiens
  source 1..22: mol_type = genomic DNA
tgattgcattggggacattgcc

SEQ ID NO: 376 - DNA, 22 bp, Homo sapiens
  source 1..22: mol_type = genomic DNA
tgttttccgggattgcattggg

SEQ ID NO: 377 - DNA, 22 bp, Homo sapiens
  source 1..22: mol_type = genomic DNA
tatctttgttttccgggattgc

SEQ ID NO: 378 - DNA, 22 bp, Homo sapiens
  source 1..22: mol_type = genomic DNA
tcaccaaatctttgttttccgg

SEQ ID NO: 379 - DNA, 22 bp, Homo sapiens
  source 1..22: mol_type = genomic DNA
ttagaaaacaccaaatctttgt

SEQ ID NO: 380 - DNA, 22 bp, Homo sapiens
  source 1..22: mol_type = genomic DNA
tgatcccaagtagaaaacacca

SEQ ID NO: 381 - DNA, 22 bp, Homo sapiens
  source 1..22: mol_type = genomic DNA
tttttgctttgtgatcccaagt

SEQ ID NO: 382 - DNA, 22 bp, Homo sapiens
  source 1..22: mol_type = genomic DNA
tgtccttttgctttgtgatccc

SEQ ID NO: 383 - DNA, 22 bp, Homo sapiens
  source 1..22: mol_type = genomic DNA
tctcatcatgccaccaccagcc

SEQ ID NO: 384 - DNA, 22 bp, Homo sapiens
  source 1..22: mol_type = genomic DNA
tcacactcatcatgccaccacc

SEQ ID NO: 385 - DNA, 22 bp, Homo sapiens
  source 1..22: mol_type = genomic DNA
ttttctccacactcatcatgcc

SEQ ID NO: 386 - DNA, 22 bp, Homo sapiens
  source 1..22: mol_type = genomic DNA
tttttctccacactcatcatgc

SEQ ID NO: 387 - DNA, 22 bp, Homo sapiens
  source 1..22: mol_type = genomic DNA
tttaggttgttttctccacact

SEQ ID NO: 388 - DNA, 22 bp, Homo sapiens
  source 1..22: mol_type = genomic DNA
tatttaggttgttttctccaca

SEQ ID NO: 389 - DNA, 22 bp, Homo sapiens
  source 1..22: mol_type = genomic DNA
ttatatttaccatttaggttgt

SEQ ID NO: 390 - DNA, 22 bp, Homo sapiens
  source 1..22: mol_type = genomic DNA
tttatatttaccatttaggttg

SEQ ID NO: 391 - DNA, 22 bp, Homo sapiens
  source 1..22: mol_type = genomic DNA
tgttatatttaccatttaggtt

SEQ ID NO: 392 - DNA, 22 bp, Homo sapiens
  source 1..22: mol_type = genomic DNA
tttgttatatttaccatttagg

SEQ ID NO: 393 - DNA, 22 bp, Homo sapiens
  source 1..22: mol_type = genomic DNA
tcttggtttgttatatttacca

SEQ ID NO: 394 - DNA, 22 bp, Homo sapiens
  source 1..22: mol_type = genomic DNA
tataaccttccattttgagact

SEQ ID NO: 395 - DNA, 22 bp, Homo sapiens
  source 1..22: mol_type = genomic DNA
tttttatagagtataaccttcc

SEQ ID NO: 396 - DNA, 22 bp, Homo sapiens
  source 1..22: mol_type = genomic DNA
tgattttatagagtataacctt

SEQ ID NO: 397 - DNA, 22 bp, Homo sapiens
  source 1..22: mol_type = genomic DNA
tttttggttgattttatagagt

SEQ ID NO: 398 - DNA, 22 bp, Homo sapiens
  source 1..22: mol_type = genomic DNA
tcattttggttgattttataga

SEQ ID NO: 399 - DNA, 22 bp, Homo sapiens
  source 1..22: mol_type = genomic DNA
tatggatcaacattttggttga

SEQ ID NO: 400 - DNA, 22 bp, Homo sapiens
  source 1..22: mol_type = genomic DNA
tctgaatctgttggatggatca

SEQ ID NO: 401 - DNA, 22 bp, Homo sapiens
  source 1..22: mol_type = genomic DNA
ttttctgaatctgttggatgga

SEQ ID NO: 402 - DNA, 22 bp, Homo sapiens
  source 1..22: mol_type = genomic DNA
tctttctgaatctgttggatgg

SEQ ID NO: 403 - DNA, 22 bp, Homo sapiens
  source 1..22: mol_type = genomic DNA
taagctttctgaatctgttgga

SEQ ID NO: 404 - DNA, 22 bp, Homo sapiens
  source 1..22: mol_type = genomic DNA
tcattcaaagctttctgaatct

SEQ ID NO: 405 - DNA, 22 bp, Homo sapiens
  source 1..22: mol_type = genomic DNA
taaatttgcctcagttcattca

SEQ ID NO: 406 - DNA, 22 bp, Homo sapiens
  source 1..22: mol_type = genomic DNA
tttttaaatttgcctcagttca

SEQ ID NO: 407 - DNA, 22 bp, Homo sapiens
  source 1..22: mol_type = genomic DNA
tcttttaaatttgcctcagttc

SEQ ID NO: 408 - DNA, 22 bp, Homo sapiens
  source 1..22: mol_type = genomic DNA
tttgccttttaaatttgcctca

SEQ ID NO: 409 - DNA, 22 bp, Homo sapiens
  source 1..22: mol_type = genomic DNA
ttattgccttttaaatttgcct

SEQ ID NO: 410 - DNA, 22 bp, Homo sapiens
  source 1..22: mol_type = genomic DNA
tttattgccttttaaatttgcc

SEQ ID NO: 411 - DNA, 22 bp, Homo sapiens
  source 1..22: mol_type = genomic DNA
tttctcttaaggatttaatacc

SEQ ID NO: 412 - DNA, 20 bp, Homo sapiens
  source 1..20: mol_type = genomic DNA
tccacgttgcttgaaattga

SEQ ID NO: 413 - DNA, 20 bp, Homo sapiens
  source 1..20: mol_type = genomic DNA
cgttgcttgaaattgaaaaa

SEQ ID NO: 414 - DNA, 20 bp, Homo sapiens
  source 1..20: mol_type = genomic DNA
taaaaatgttcacaattaaa

SEQ ID NO: 415 - DNA, 20 bp, Homo sapiens
  source 1..20: mol_type = genomic DNA
aaaaatgttcacaattaaga

SEQ ID NO: 416 - DNA, 20 bp, Homo sapiens
  source 1..20: mol_type = genomic DNA
ttcctctagttatttcctca

SEQ ID NO: 417 - DNA, 20 bp, Homo sapiens
  source 1..20: mol_type = genomic DNA
ctccagagccaaaatcaaga

SEQ ID NO: 418 - DNA, 20 bp, Homo sapiens
  source 1..20: mol_type = genomic DNA
atgttagacgatgtaaaaaa

SEQ ID NO: 419 - DNA, 20 bp, Homo sapiens
  source 1..20: mol_type = genomic DNA
agacgatgtaaaaattttaa

SEQ ID NO: 420 - DNA, 20 bp, Homo sapiens
  source 1..20: mol_type = genomic DNA
gggacatggtcttaaagaca

SEQ ID NO: 421 - DNA, 20 bp, Homo sapiens
  source 1..20: mol_type = genomic DNA
ggacatggtcttaaagacta

SEQ ID NO: 422 - DNA, 20 bp, Homo sapiens
  source 1..20: mol_type = genomic DNA
catggtcttaaagactttga

SEQ ID NO: 423 - DNA, 20 bp, Homo sapiens
  source 1..20: mol_type = genomic DNA
aagactttgtccataagaca

SEQ ID NO: 424 - DNA, 20 bp, Homo sapiens
  source 1..20: mol_type = genomic DNA
taatgacatatttcaaaaaa

SEQ ID NO: 425 - DNA, 20 bp, Homo sapiens
  source 1..20: mol_type = genomic DNA
aatgacatatttcaaaaaca

SEQ ID NO: 426 - DNA, 20 bp, Homo sapiens
  source 1..20: mol_type = genomic DNA
tcaaaaactcaacatattta

SEQ ID NO: 427 - DNA, 20 bp, Homo sapiens
  source 1..20: mol_type = genomic DNA
aaaactcaacatatttgata

SEQ ID NO: 428 - DNA, 20 bp, Homo sapiens
  source 1..20: mol_type = genomic DNA
gatcagtctttttatgatca

SEQ ID NO: 429 - DNA, 20 bp, Homo sapiens
  source 1..20: mol_type = genomic DNA
gcaaaccagtgaaatcaaaa

SEQ ID NO: 430 - DNA, 20 bp, Homo sapiens
  source 1..20: mol_type = genomic DNA
aaccagtgaaatcaaagaaa

SEQ ID NO: 431 - DNA, 20 bp, Homo sapiens
  source 1..20: mol_type = genomic DNA
gaaatcaaagaagaagaaaa

SEQ ID NO: 432 - DNA, 20 bp, Homo sapiens
  source 1..20: mol_type = genomic DNA
aatcaaagaagaagaaaaga

SEQ ID NO: 433 - DNA, 20 bp, Homo sapiens
  source 1..20: mol_type = genomic DNA
aggaactgagaagaactaca

SEQ ID NO: 434 - DNA, 20 bp, Homo sapiens
  source 1..20: mol_type = genomic DNA
actgagaagaactacatata

SEQ ID NO: 435 - DNA, 20 bp, Homo sapiens
  source 1..20: mol_type = genomic DNA
actacatataaactacaaga

SEQ ID NO: 436 - DNA, 20 bp, Homo sapiens
  source 1..20: mol_type = genomic DNA
tacatataaactacaagtca

SEQ ID NO: 437 - DNA, 20 bp, Homo sapiens
  source 1..20: mol_type = genomic DNA
tataaactacaagtcaaaaa

SEQ ID NO: 438 - DNA, 20 bp, Homo sapiens
  source 1..20: mol_type = genomic DNA
aaactacaagtcaaaaatga

SEQ ID NO: 439 - DNA, 20 bp, Homo sapiens
  source 1..20: mol_type = genomic DNA
actacaagtcaaaaatgaaa

SEQ ID NO: 440 - DNA, 20 bp, Homo sapiens
  source 1..20: mol_type = genomic DNA
aaatgaagaggtaaagaata

SEQ ID NO: 441 - DNA, 20 bp, Homo sapiens
  source 1..20: mol_type = genomic DNA
tgaagaggtaaagaatatga

SEQ ID NO: 442 - DNA, 20 bp, Homo sapiens
  source 1..20: mol_type = genomic DNA
cttgaactcaactcaaaaca

SEQ ID NO: 443 - DNA, 20 bp, Homo sapiens
  source 1..20: mol_type = genomic DNA
tgaaagcctcctagaagaaa

SEQ ID NO: 444 - DNA, 20 bp, Homo sapiens
  source 1..20: mol_type = genomic DNA
gaaagcctcctagaagaaaa

SEQ ID NO: 445 - DNA, 20 bp, Homo sapiens
  source 1..20: mol_type = genomic DNA
aagcctcctagaagaaaaaa

SEQ ID NO: 446 - DNA, 20 bp, Homo sapiens
  source 1..20: mol_type = genomic DNA
agcctcctagaagaaaaaaa

SEQ ID NO: 447 - DNA, 20 bp, Homo sapiens
  source 1..20: mol_type = genomic DNA
tcctagaagaaaaaattcta

SEQ ID NO: 448 - DNA, 20 bp, Homo sapiens
  source 1..20: mol_type = genomic DNA
aaaattctacttcaacaaaa

SEQ ID NO: 449 - DNA, 20 bp, Homo sapiens
  source 1..20: mol_type = genomic DNA
aaattctacttcaacaaaaa

SEQ ID NO: 450 - DNA, 20 bp, Homo sapiens
  source 1..20: mol_type = genomic DNA
aattctacttcaacaaaaaa

SEQ ID NO: 451 - DNA, 20 bp, Homo sapiens
  source 1..20: mol_type = genomic DNA
attctacttcaacaaaaaga

SEQ ID NO: 452 - DNA, 20 bp, Homo sapiens
  source 1..20: mol_type = genomic DNA
cttcaacaaaaagtgaaata

SEQ ID NO: 453 - DNA, 20 bp, Homo sapiens
  source 1..20: mol_type = genomic DNA
tcaacaaaaagtgaaatata

SEQ ID NO: 454 - DNA, 20 bp, Homo sapiens
  source 1..20: mol_type = genomic DNA
aaagtgaaatatttagaaga

SEQ ID NO: 455 - DNA, 20 bp, Homo sapiens
  source 1..20: mol_type = genomic DNA
gcaactaactaacttaatta

SEQ ID NO: 456 - DNA, 20 bp, Homo sapiens
  source 1..20: mol_type = genomic DNA
aactaacttaattcaaaata

SEQ ID NO: 457 - DNA, 20 bp, Homo sapiens
  source 1..20: mol_type = genomic DNA
actaacttaattcaaaatca

SEQ ID NO: 458 - DNA, 20 bp, Homo sapiens
  source 1..20: mol_type = genomic DNA
aacttaattcaaaatcaaca

SEQ ID NO: 459 - DNA, 20 bp, Homo sapiens
  source 1..20: mol_type = genomic DNA
aaaccaacagcatagtcaaa

SEQ ID NO: 460 - DNA, 20 bp, Homo sapiens
  source 1..20: mol_type = genomic DNA
aaccaacagcatagtcaaaa

SEQ ID NO: 461 - DNA, 20 bp, Homo sapiens
  source 1..20: mol_type = genomic DNA
ccaacagcatagtcaaataa

SEQ ID NO: 462 - DNA, 20 bp, Homo sapiens
  source 1..20: mol_type = genomic DNA
acagcatagtcaaataaaaa

SEQ ID NO: 463 - DNA, 20 bp, Homo sapiens
  source 1..20: mol_type = genomic DNA
gcatagtcaaataaaagaaa

SEQ ID NO: 464 - DNA, 20 bp, Homo sapiens
  source 1..20: mol_type = genomic DNA
tagtcaaataaaagaaataa

SEQ ID NO: 465 - DNA, 20 bp, Homo sapiens
  source 1..20: mol_type = genomic DNA
agtcaaataaaagaaataga

SEQ ID NO: 466 - DNA, 20 bp, Homo sapiens
  source 1..20: mol_type = genomic DNA
caaataaaagaaatagaaaa

SEQ ID NO: 467 - DNA, 20 bp, Homo sapiens
  source 1..20: mol_type = genomic DNA
aataaaagaaatagaaaata

SEQ ID NO: 468 - DNA, 20 bp, Homo sapiens
  source 1..20: mol_type = genomic DNA
aaaagaaatagaaaatcaga

SEQ ID NO: 469 - DNA, 20 bp, Homo sapiens
  source 1..20: mol_type = genomic DNA
caagaacccacagaaattta

SEQ ID NO: 470 - DNA, 20 bp, Homo sapiens
  source 1..20: mol_type = genomic DNA
agaaatttctctatcttcca

SEQ ID NO: 471 - DNA, 20 bp, Homo sapiens
  source 1..20: mol_type = genomic DNA
tgaaataagaaatgtaaaaa

SEQ ID NO: 472 - DNA, 20 bp, Homo sapiens
  source 1..20: mol_type = genomic DNA
gaaataagaaatgtaaaaca

SEQ ID NO: 473 - DNA, 20 bp, Homo sapiens
  source 1..20: mol_type = genomic DNA
aataagaaatgtaaaacata

SEQ ID NO: 474 - DNA, 20 bp, Homo sapiens
  source 1..20: mol_type = genomic DNA
aagaaatgtaaaacatgata

SEQ ID NO: 475 - DNA, 20 bp, Homo sapiens
  source 1..20: mol_type = genomic DNA
aactctcaagtttttcatga

SEQ ID NO: 476 - DNA, 20 bp, Homo sapiens
  source 1..20: mol_type = genomic DNA
tagatggatcacaaaactta

SEQ ID NO: 477 - DNA, 20 bp, Homo sapiens
  source 1..20: mol_type = genomic DNA
atcacaaaacttcaatgaaa

SEQ ID NO: 478 - DNA, 20 bp, Homo sapiens
  source 1..20: mol_type = genomic DNA
cacaaaacttcaatgaaaca

SEQ ID NO: 479 - DNA, 20 bp, Homo sapiens
  source 1..20: mol_type = genomic DNA
acaaaacttcaatgaaacga

SEQ ID NO: 480 - DNA, 20 bp, Homo sapiens
  source 1..20: mol_type = genomic DNA
aacgtgggagaactacaaaa

SEQ ID NO: 481 - DNA, 20 bp, Homo sapiens
  source 1..20: mol_type = genomic DNA
acgtgggagaactacaaata

SEQ ID NO: 482 - DNA, 20 bp, Homo sapiens
  source 1..20: mol_type = genomic DNA
cgtgggagaactacaaataa

SEQ ID NO: 483 - DNA, 20 bp, Homo sapiens
  source 1..20: mol_type = genomic DNA
gggcctagagaagatataca

SEQ ID NO: 484 - DNA, 20 bp, Homo sapiens
  source 1..20: mol_type = genomic DNA
ggcctagagaagatatacta

SEQ ID NO: 485 - DNA, 20 bp, Homo sapiens
  source 1..20: mol_type = genomic DNA
gaagactggaaagacaacaa

SEQ ID NO: 486 - DNA, 20 bp, Homo sapiens
  source 1..20: mol_type = genomic DNA
gactggaaagacaacaaaca

SEQ ID NO: 487 - DNA, 20 bp, Homo sapiens
  source 1..20: mol_type = genomic DNA
ctggaaagacaacaaacata

SEQ ID NO: 488 - DNA, 20 bp, Homo sapiens
  source 1..20: mol_type = genomic DNA
agacaacaaacattatatta

SEQ ID NO: 489 - DNA, 20 bp, Homo sapiens
  source 1..20: mol_type = genomic DNA
caacaaacattatattgaaa

SEQ ID NO: 490 - DNA, 20 bp, Homo sapiens
  source 1..20: mol_type = genomic DNA
acaaacattatattgaataa

SEQ ID NO: 491 - DNA, 20 bp, Homo sapiens
  source 1..20: mol_type = genomic DNA
caaacattatattgaatata

SEQ ID NO: 492 - DNA, 20 bp, Homo sapiens
  source 1..20: mol_type = genomic DNA
cattatattgaatattctta

SEQ ID NO: 493 - DNA, 20 bp, Homo sapiens
  source 1..20: mol_type = genomic DNA
atattgaatattctttttaa

SEQ ID NO: 494 - DNA, 20 bp, Homo sapiens
  source 1..20: mol_type = genomic DNA
tattgaatattctttttaca

SEQ ID NO: 495 - DNA, 20 bp, Homo sapiens
  source 1..20: mol_type = genomic DNA
ggaaatcacgaaaccaacta

SEQ ID NO: 496 - DNA, 20 bp, Homo sapiens
  source 1..20: mol_type = genomic DNA
tgcaatcccggaaaacaaaa

SEQ ID NO: 497 - DNA, 20 bp, Homo sapiens
  source 1..20: mol_type = genomic DNA
gcaatcccggaaaacaaaga

SEQ ID NO: 498 - DNA, 20 bp, Homo sapiens
  source 1..20: mol_type = genomic DNA
ccggaaaacaaagatttgga

SEQ ID NO: 499 - DNA, 20 bp, Homo sapiens
  source 1..20: mol_type = genomic DNA
aagatttggtgttttctaca

SEQ ID NO: 500 - DNA, 20 bp, Homo sapiens
  source 1..20: mol_type = genomic DNA
atttggtgttttctacttga

SEQ ID NO: 501 - DNA, 20 bp, Homo sapiens
  source 1..20: mol_type = genomic DNA
tctacttgggatcacaaaga

SEQ ID NO: 502 - DNA, 20 bp, Homo sapiens
  source 1..20: mol_type = genomic DNA
tgggatcacaaagcaaaaga

SEQ ID NO: 503 - DNA, 20 bp, Homo sapiens
  source 1..20: mol_type = genomic DNA
gatgagtgtggagaaaacaa

SEQ ID NO: 504 - DNA, 20 bp, Homo sapiens
  source 1..20: mol_type = genomic DNA
atgagtgtggagaaaacaaa

SEQ ID NO: 505 - DNA, 20 bp, Homo sapiens
  source 1..20: mol_type = genomic DNA
tgagtgtggagaaaacaaca

SEQ ID NO: 506 - DNA, 20 bp, Homo sapiens
  source 1..20: mol_type = genomic DNA
ggagaaaacaacctaaatga

SEQ ID NO: 507 - DNA, 20 bp, Homo sapiens
  source 1..20: mol_type = genomic DNA
aaaacaacctaaatggtaaa

SEQ ID NO: 508 - DNA, 20 bp, Homo sapiens
  source 1..20: mol_type = genomic DNA
aaacaacctaaatggtaaaa

SEQ ID NO: 509 - DNA, 20 bp, Homo sapiens
  source 1..20: mol_type = genomic DNA
aacaacctaaatggtaaata

SEQ ID NO: 510 - DNA, 20 bp, Homo sapiens
  source 1..20: mol_type = genomic DNA
caacctaaatggtaaatata

SEQ ID NO: 511 - DNA, 20 bp, Homo sapiens
  source 1..20: mol_type = genomic DNA
ctaaatggtaaatataacaa

SEQ ID NO: 512 - DNA, 20 bp, Homo sapiens
  source 1..20: mol_type = genomic DNA
aaatggtaaatataacaaaa

SEQ ID NO: 513 - DNA, 20 bp, Homo sapiens
  source 1..20: mol_type = genomic DNA
aatggtaaatataacaaaca

SEQ ID NO: 514 - DNA, 20 bp, Homo sapiens
  source 1..20: mol_type = genomic DNA
aaaatggaaggttatactca

SEQ ID NO: 515 - DNA, 20 bp, Homo sapiens
  source 1..20: mol_type = genomic DNA
tggaaggttatactctataa

SEQ ID NO: 516 - DNA, 20 bp, Homo sapiens
  source 1..20: mol_type = genomic DNA
aggttatactctataaaata

SEQ ID NO: 517 - DNA, 20 bp, Homo sapiens
  source 1..20: mol_type = genomic DNA
tatactctataaaatcaaca

SEQ ID NO: 518 - DNA, 20 bp, Homo sapiens
  source 1..20: mol_type = genomic DNA
ctataaaatcaaccaaaata

SEQ ID NO: 519 - DNA, 20 bp, Homo sapiens
  source 1..20: mol_type = genomic DNA
acagattcagaaagctttga

SEQ ID NO: 520 - DNA, 20 bp, Homo sapiens
  source 1..20: mol_type = genomic DNA
agaaagctttgaatgaacta

SEQ ID NO: 521 - DNA, 20 bp, Homo sapiens
  source 1..20: mol_type = genomic DNA
tgaactgaggcaaatttaaa

SEQ ID NO: 522 - DNA, 20 bp, Homo sapiens
  source 1..20: mol_type = genomic DNA
gaactgaggcaaatttaaaa

SEQ ID NO: 523 - DNA, 22 bp, Homo sapiens
  source 1..22: mol_type = genomic DNA
tcaatttcaagcaacgtggaac

SEQ ID NO: 524 - DNA, 22 bp, Homo sapiens
  source 1..22: mol_type = genomic DNA
tttttcaatttcaagcaacgtg

SEQ ID NO: 525 - DNA, 22 bp, Homo sapiens
  source 1..22: mol_type = genomic DNA
tttaattgtgaacatttttatc

SEQ ID NO: 526 - DNA, 22 bp, Homo sapiens
  source 1..22: mol_type = genomic DNA
tcttaattgtgaacatttttat

SEQ ID NO: 527 - DNA, 22 bp, Homo sapiens
  source 1..22: mol_type = genomic DNA
tgaggaaataactagaggaaca

SEQ ID NO: 528 - DNA, 22 bp, Homo sapiens
  source 1..22: mol_type = genomic DNA
tcttgattttggctctggagat

SEQ ID NO: 529 - DNA, 22 bp, Homo sapiens
  source 1..22: mol_type = genomic DNA
ttttttacatcgtctaacatag

SEQ ID NO: 530 - DNA, 22 bp, Homo sapiens
  source 1..22: mol_type = genomic DNA
ttaaaatttttacatcgtctaa

SEQ ID NO: 531 - DNA, 22 bp, Homo sapiens
  source 1..22: mol_type = genomic DNA
tgtctttaagaccatgtcccaa

SEQ ID NO: 532 - DNA, 22 bp, Homo sapiens
  source 1..22: mol_type = genomic DNA
tagtctttaagaccatgtccca

SEQ ID NO: 533 - DNA, 22 bp, Homo sapiens
  source 1..22: mol_type = genomic DNA
tcaaagtctttaagaccatgtc

SEQ ID NO: 534 - DNA, 22 bp, Homo sapiens
  source 1..22: mol_type = genomic DNA
tgtcttatggacaaagtcttta

SEQ ID NO: 535 - DNA, 22 bp, Homo sapiens
  source 1..22: mol_type = genomic DNA
ttttttgaaatatgtcattaat

SEQ ID NO: 536 - DNA, 22 bp, Homo sapiens
  source 1..22: mol_type = genomic DNA
tgtttttgaaatatgtcattaa

SEQ ID NO: 537 - DNA, 22 bp, Homo sapiens
  source 1..22: mol_type = genomic DNA
taaatatgttgagtttttgaaa

SEQ ID NO: 538 - DNA, 22 bp, Homo sapiens
  source 1..22: mol_type = genomic DNA
tatcaaatatgttgagtttttg

SEQ ID NO: 539 - DNA, 22 bp, Homo sapiens
  source 1..22: mol_type = genomic DNA
tgatcataaaaagactgatcaa

SEQ ID NO: 540 - DNA, 22 bp, Homo sapiens
  source 1..22: mol_type = genomic DNA
ttttgatttcactggtttgcag

SEQ ID NO: 541 - DNA, 22 bp, Homo sapiens
  source 1..22: mol_type = genomic DNA
tttctttgatttcactggtttg

SEQ ID NO: 542 - DNA, 22 bp, Homo sapiens
  source 1..22: mol_type = genomic DNA
ttttcttcttctttgatttcac

SEQ ID NO: 543 - DNA, 22 bp, Homo sapiens
  source 1..22: mol_type = genomic DNA
tcttttcttcttctttgatttc

SEQ ID NO: 544 - DNA, 22 bp, Homo sapiens
  source 1..22: mol_type = genomic DNA
tgtagttcttctcagttccttt

SEQ ID NO: 545 - DNA, 22 bp, Homo sapiens
  source 1..22: mol_type = genomic DNA
tatatgtagttcttctcagttc

SEQ ID NO: 546 - DNA, 22 bp, Homo sapiens
  source 1..22: mol_type = genomic DNA
tcttgtagtttatatgtagttc

SEQ ID NO: 547 - DNA, 22 bp, Homo sapiens
  source 1..22: mol_type = genomic DNA
tgacttgtagtttatatgtagt

SEQ ID NO: 548 - DNA, 22 bp, Homo sapiens
  source 1..22: mol_type = genomic DNA
tttttgacttgtagtttatatg

SEQ ID NO: 549 - DNA, 22 bp, Homo sapiens
  source 1..22: mol_type = genomic DNA
tcatttttgacttgtagtttat

SEQ ID NO: 550 - DNA, 22 bp, Homo sapiens
  source 1..22: mol_type = genomic DNA
tttcatttttgacttgtagttt

SEQ ID NO: 551 - DNA, 22 bp, Homo sapiens
  source 1..22: mol_type = genomic DNA
tattctttacctcttcattttt

SEQ ID NO: 552 - DNA, 22 bp, Homo sapiens
  source 1..22: mol_type = genomic DNA
tcatattctttacctcttcatt

SEQ ID NO: 553 - DNA, 22 bp, Homo sapiens
  source 1..22: mol_type = genomic DNA
tgttttgagttgagttcaagtg

SEQ ID NO: 554 - DNA, 22 bp, Homo sapiens
  source 1..22: mol_type = genomic DNA
tttcttctaggaggctttcaag

SEQ ID NO: 555 - DNA, 22 bp, Homo sapiens
  source 1..22: mol_type = genomic DNA
ttttcttctaggaggctttcaa

SEQ ID NO: 556 - DNA, 22 bp, Homo sapiens
  source 1..22: mol_type = genomic DNA
ttttttcttctaggaggctttc

SEQ ID NO: 557 - DNA, 22 bp, Homo sapiens
  source 1..22: mol_type = genomic DNA
tttttttcttctaggaggcttt

SEQ ID NO: 558 - DNA, 22 bp, Homo sapiens
  source 1..22: mol_type = genomic DNA
tagaattttttcttctaggagg

SEQ ID NO: 559 - DNA, 22 bp, Homo sapiens
  source 1..22: mol_type = genomic DNA
ttttgttgaagtagaatttttt

SEQ ID NO: 560 - DNA, 22 bp, Homo sapiens
  source 1..22: mol_type = genomic DNA
tttttgttgaagtagaattttt

SEQ ID NO: 561 - DNA, 22 bp, Homo sapiens
  source 1..22: mol_type = genomic DNA
ttttttgttgaagtagaatttt

SEQ ID NO: 562 - DNA, 22 bp, Homo sapiens
  source 1..22: mol_type = genomic DNA
tctttttgttgaagtagaattt

SEQ ID NO: 563 - DNA, 22 bp, Homo sapiens
  source 1..22: mol_type = genomic DNA
tatttcactttttgttgaagta

SEQ ID NO: 564 - DNA, 22 bp, Homo sapiens
  source 1..22: mol_type = genomic DNA
tatatttcactttttgttgaag

SEQ ID NO: 565 - DNA, 22 bp, Homo sapiens
  source 1..22: mol_type = genomic DNA
tcttctaaatatttcacttttt

SEQ ID NO: 566 - DNA, 22 bp, Homo sapiens
  source 1..22: mol_type = genomic DNA
taattaagttagttagttgctc

SEQ ID NO: 567 - DNA, 22 bp, Homo sapiens
  source 1..22: mol_type = genomic DNA
tattttgaattaagttagttag

SEQ ID NO: 568 - DNA, 22 bp, Homo sapiens
  source 1..22: mol_type = genomic DNA
tgattttgaattaagttagtta

SEQ ID NO: 569 - DNA, 22 bp, Homo sapiens
  source 1..22: mol_type = genomic DNA
tgttgattttgaattaagttag

SEQ ID NO: 570 - DNA, 22 bp, Homo sapiens
  source 1..22: mol_type = genomic DNA
tttgactatgctgttggtttaa

SEQ ID NO: 571 - DNA, 22 bp, Homo sapiens
  source 1..22: mol_type = genomic DNA
ttttgactatgctgttggttta

SEQ ID NO: 572 - DNA, 22 bp, Homo sapiens
  source 1..22: mol_type = genomic DNA
ttatttgactatgctgttggtt

SEQ ID NO: 573 - DNA, 22 bp, Homo sapiens
  source 1..22: mol_type = genomic DNA
tttttatttgactatgctgttg

SEQ ID NO: 574 - DNA, 22 bp, Homo sapiens
  source 1..22: mol_type = genomic DNA
tttcttttatttgactatgctg

SEQ ID NO: 575 - DNA, 22 bp, Homo sapiens
  source 1..22: mol_type = genomic DNA
ttatttcttttatttgactatg

SEQ ID NO: 576 - DNA, 22 bp, Homo sapiens
  source 1..22: mol_type = genomic DNA
tctatttcttttatttgactat

SEQ ID NO: 577 - DNA, 22 bp, Homo sapiens
  source 1..22: mol_type = genomic DNA
ttttctatttcttttatttgac

SEQ ID NO: 578 - DNA, 22 bp, Homo sapiens
  source 1..22: mol_type = genomic DNA
tattttctatttcttttatttg

SEQ ID NO: 579 - DNA, 22 bp, Homo sapiens
  source 1..22: mol_type = genomic DNA
tctgattttctatttcttttat

SEQ ID NO: 580 - DNA, 22 bp, Homo sapiens
  source 1..22: mol_type = genomic DNA
taaatttctgtgggttcttgaa

SEQ ID NO: 581 - DNA, 22 bp, Homo sapiens
  source 1..22: mol_type = genomic DNA
tggaagatagagaaatttctgt

SEQ ID NO: 582 - DNA, 22 bp, Homo sapiens
  source 1..22: mol_type = genomic DNA
tttttacatttcttatttcatt

SEQ ID NO: 583 - DNA, 22 bp, Homo sapiens
  source 1..22: mol_type = genomic DNA
tgttttacatttcttatttcat

SEQ ID NO: 584 - DNA, 22 bp, Homo sapiens
  source 1..22: mol_type = genomic DNA
tatgttttacatttcttatttc

SEQ ID NO: 585 - DNA, 22 bp, Homo sapiens
  source 1..22: mol_type = genomic DNA
tatcatgttttacatttcttat

SEQ ID NO: 586 - DNA, 22 bp, Homo sapiens
  source 1..22: mol_type = genomic DNA
tcatgaaaaacttgagagttgc

SEQ ID NO: 587 - DNA, 22 bp, Homo sapiens
  source 1..22: mol_type = genomic DNA
taagttttgtgatccatctatt

SEQ ID NO: 588 - DNA, 22 bp, Homo sapiens
  source 1..22: mol_type = genomic DNA
tttcattgaagttttgtgatcc

SEQ ID NO: 589 - DNA, 22 bp, Homo sapiens
  source 1..22: mol_type = genomic DNA
tgtttcattgaagttttgtgat

SEQ ID NO: 590 - DNA, 22 bp, Homo sapiens
  source 1..22: mol_type = genomic DNA
tcgtttcattgaagttttgtga

SEQ ID NO: 591 - DNA, 22 bp, Homo sapiens
  source 1..22: mol_type = genomic DNA
ttttgtagttctcccacgtttc

SEQ ID NO: 592 - DNA, 22 bp, Homo sapiens
  source 1..22: mol_type = genomic DNA
tatttgtagttctcccacgttt

SEQ ID NO: 593 - DNA, 22 bp, Homo sapiens
  source 1..22: mol_type = genomic DNA
ttatttgtagttctcccacgtt

SEQ ID NO: 594 - DNA, 22 bp, Homo sapiens
  source 1..22: mol_type = genomic DNA
tgtatatcttctctaggcccaa

SEQ ID NO: 595 - DNA, 22 bp, Homo sapiens
  source 1..22: mol_type = genomic DNA
tagtatatcttctctaggccca

SEQ ID NO: 596 - DNA, 22 bp, Homo sapiens
  source 1..22: mol_type = genomic DNA
ttgttgtctttccagtcttcca

SEQ ID NO: 597 - DNA, 22 bp, Homo sapiens
  source 1..22: mol_type = genomic DNA
tgtttgttgtctttccagtctt

SEQ ID NO: 598 - DNA, 22 bp, Homo sapiens
  source 1..22: mol_type = genomic DNA
tatgtttgttgtctttccagtc

SEQ ID NO: 599 - DNA, 22 bp, Homo sapiens
  source 1..22: mol_type = genomic DNA
taatataatgtttgttgtcttt

SEQ ID NO: 600 - DNA, 22 bp, Homo sapiens
  source 1..22: mol_type = genomic DNA
tttcaatataatgtttgttgtc

SEQ ID NO: 601 - DNA, 22 bp, Homo sapiens
  source 1..22: mol_type = genomic DNA
ttattcaatataatgtttgttg

SEQ ID NO: 602 - DNA, 22 bp, Homo sapiens
  source 1..22: mol_type = genomic DNA
tatattcaatataatgtttgtt

SEQ ID NO: 603 - DNA, 22 bp, Homo sapiens
  source 1..22: mol_type = genomic DNA
taagaatattcaatataatgtt

SEQ ID NO: 604 - DNA, 22 bp, Homo sapiens
  source 1..22: mol_type = genomic DNA
ttaaaaagaatattcaatataa

SEQ ID NO: 605 - DNA, 22 bp, Homo sapiens
  source 1..22: mol_type = genomic DNA
tgtaaaaagaatattcaatata

SEQ ID NO: 606 - DNA, 22 bp, Homo sapiens
  source 1..22: mol_type = genomic DNA
tagttggtttcgtgatttccca

SEQ ID NO: 607 - DNA, 22 bp, Homo sapiens
  source 1..22: mol_type = genomic DNA
ttttgttttccgggattgcatt

SEQ ID NO: 608 - DNA, 22 bp, Homo sapiens
  source 1..22: mol_type = genomic DNA
tctttgttttccgggattgcat

SEQ ID NO: 609 - DNA, 22 bp, Homo sapiens
  source 1..22: mol_type = genomic DNA
tccaaatctttgttttccggga

SEQ ID NO: 610 - DNA, 22 bp, Homo sapiens
  source 1..22: mol_type = genomic DNA
tgtagaaaacaccaaatctttg

SEQ ID NO: 611 - DNA, 22 bp, Homo sapiens
  source 1..22: mol_type = genomic DNA
tcaagtagaaaacaccaaatct

SEQ ID NO: 612 - DNA, 22 bp, Homo sapiens
  source 1..22: mol_type = genomic DNA
tctttgtgatcccaagtagaaa

SEQ ID NO: 613 - DNA, 22 bp, Homo sapiens
  source 1..22: mol_type = genomic DNA
tcttttgctttgtgatcccaag

SEQ ID NO: 614 - DNA, 22 bp, Homo sapiens
  source 1..22: mol_type = genomic DNA
ttgttttctccacactcatcat

SEQ ID NO: 615 - DNA, 22 bp, Homo sapiens
  source 1..22: mol_type = genomic DNA
tttgttttctccacactcatca

SEQ ID NO: 616 - DNA, 22 bp, Homo sapiens
  source 1..22: mol_type = genomic DNA
tgttgttttctccacactcatc

SEQ ID NO: 617 - DNA, 22 bp, Homo sapiens
  source 1..22: mol_type = genomic DNA
tcatttaggttgttttctccac

SEQ ID NO: 618 - DNA, 22 bp, Homo sapiens
  source 1..22: mol_type = genomic DNA
tttaccatttaggttgttttct

SEQ ID NO: 619 - DNA, 22 bp, Homo sapiens
  source 1..22: mol_type = genomic DNA
ttttaccatttaggttgttttc

SEQ ID NO: 620 - DNA, 22 bp, Homo sapiens
  source 1..22: mol_type = genomic DNA
tatttaccatttaggttgtttt

SEQ ID NO: 621 - DNA, 22 bp, Homo sapiens
  source 1..22: mol_type = genomic DNA
tatatttaccatttaggttgtt

SEQ ID NO: 622 - DNA, 22 bp, Homo sapiens
  source 1..22: mol_type = genomic DNA
ttgttatatttaccatttaggt

SEQ ID NO: 623 - DNA, 22 bp, Homo sapiens
  source 1..22: mol_type = genomic DNA
ttttgttatatttaccatttag

SEQ ID NO: 624 - DNA, 22 bp, Homo sapiens
  source 1..22: mol_type = genomic DNA
tgtttgttatatttaccattta

SEQ ID NO: 625 - DNA, 22 bp, Homo sapiens
  source 1..22: mol_type = genomic DNA
tgagtataaccttccattttga

SEQ ID NO: 626 - DNA, 22 bp, Homo sapiens
  source 1..22: mol_type = genomic DNA
ttatagagtataaccttccatt

SEQ ID NO: 627 - DNA, 22 bp, Homo sapiens
  source 1..22: mol_type = genomic DNA
tattttatagagtataaccttc

SEQ ID NO: 628 - DNA, 22 bp, Homo sapiens
  source 1..22: mol_type = genomic DNA
tgttgattttatagagtataac

SEQ ID NO: 629 - DNA, 22 bp, Homo sapiens
  source 1..22: mol_type = genomic DNA
tattttggttgattttatagag

SEQ ID NO: 630 - DNA, 22 bp, Homo sapiens
  source 1..22: mol_type = genomic DNA
tcaaagctttctgaatctgttg

SEQ ID NO: 631 - DNA, 22 bp, Homo sapiens
  source 1..22: mol_type = genomic DNA
tagttcattcaaagctttctga

SEQ ID NO: 632 - DNA, 22 bp, Homo sapiens
  source 1..22: mol_type = genomic DNA
tttaaatttgcctcagttcatt

SEQ ID NO: 633 - DNA, 22 bp, Homo sapiens
  source 1..22: mol_type = genomic DNA
ttttaaatttgcctcagttcat

SEQ ID NO: 634 - DNA, 20 bp, Homo sapiens
  source 1..20: mol_type = genomic DNA
cagcaactctcaagttttta

SEQ ID NO: 635 - DNA, 20 bp, Homo sapiens
  source 1..20: mol_type = genomic DNA
aaagatttggtgttttctaa